(19) United States
(12) Patent Application Publication
(10) Pub. No.: US 2025/0386664 A1
(43) Pub. Date: Dec. 18, 2025

(54) COMPOUNDS FOR ELECTRONIC DEVICES

(71) Applicant: Merck Patent GmbH, Darmstadt (DE)

(72) Inventors: Rouven LINGE, Darmstadt (DE); Elvira MONTENEGRO, Darmstadt (DE); Jens ENGELHART, Darmstadt (DE); Teresa MUJICA-FERNAUD, Darmstadt (DE)

(21) Appl. No.: 18/878,154

(22) PCT Filed: Jun. 6, 2023
(86) PCT No.: PCT/EP2023/065029

§ 371 Date: Dec. 23, 2024

(30) Foreign Application Priority Data
Jun. 28, 2022 (EP) 22181673.9

(51) Int. Cl. H10K 50/155; C09K 11/06; H10K 50/17; H10K 50/18; H10K 85/30; H10K 85/60

(52) U.S. Cl. CPC H10K 50/155; C09K 11/06; H10K 50/17; H10K 50/181; H10K 85/342; H10K 85/346; H10K 85/622; H10K 85/624; H10K 85/626; H10K 85/633; H10K 85/636; H10K 85/654; H10K 85/6572; H10K 85/6574; H10K 85/6576; C09K 2211/10

(57) ABSTRACT

The invention relates to compounds of the formula (I), to methods for producing compounds of the formula (I), to the use of compounds of the formula (I) in electronic devices, and to electronic devices containing a compound of the formula (I).

The present application relates to aromatic amines having particular aromatic or heteroaromatic ring systems on the amine nitrogen atom. The compounds are suitable for use in electronic devices.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The structure and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having a hole-transporting function. There is an ongoing search for novel compounds for use in these layers, especially hole-transporting compounds and compounds that can serve as hole-transporting matrix material, especially for phosphorescent emitters, in an emitting layer. For this purpose, there is a search in particular for compounds that have a high glass transition temperature, high stability, and high conductivity for holes. A high stability of the compound is a prerequisite for achieving a long lifetime of the electronic device. In addition, a sufficiently low sublimation temperature is of interest and is preferred in order to be able to produce electronic devices comprising the compound by means of vapor deposition methods. In addition, a sufficiently high HOMO of the compounds is of interest and is preferred. There is also a search for compounds whose use in electronic devices results in improvement of the performance data of the devices, especially in high efficiency, long lifetime and low operating voltage.

In the prior art, triarylamine compounds in particular, for example spirobifluoreneamines and fluoreneamines, are known as hole transport materials and hole-transporting matrix materials for electronic devices. However, there remains room for improvement in respect of the abovementioned properties.

It has now been found that aromatic amines of the formulae below which are characterized in that they have particular aromatic or heteroaromatic ring systems on the amine nitrogen atom are of excellent suitability for use in electronic devices.

They are especially suitable for use in OLEDs, and even more particularly therein for use as hole transport materials and for use as hole-transporting matrix materials, especially for phosphorescent emitters. The compounds lead to high lifetime, high efficiency and low operating voltage of the devices. Further preferably, the compounds found have a high glass transition temperature, high stability, low sublimation temperature, good solubility, good synthetic accessibility, a sufficiently high HOMO and high conductivity for holes.

The present application provides an electronic device comprising anode, cathode, an emitting layer, and a layer comprising a compound of the formula (I) disposed between the anode and the emitting layer,

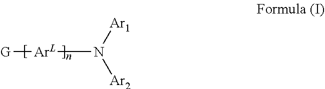

where the variables that occur are as follows:
G is a group of formula (G) which is bonded to the remainder of the formula (I) via one of the unoccupied positions on the benzene rings, where the other unoccupied positions on the benzene rings are each substituted by an R¹ radical;

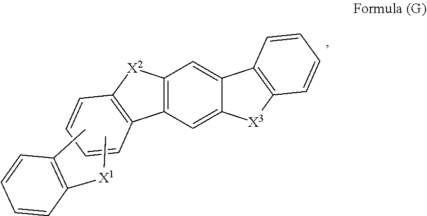

X¹ is O or S;
X² is C(R²)₂ or Si(R²)₂;
X³ is C(R²)₂ or Si(R²)₂;
Ar^{L} is an aromatic ring system which has 6 to 40 aromatic ring atoms and is substituted by R³ radicals, or a heteroaromatic ring system which has 5 to 40 aromatic ring atoms and is substituted by R³ radicals;
Ar¹, Ar² is the same or different and is an aromatic ring system which has 6 to 40 aromatic ring atoms and is substituted by R⁴ radicals, or a heteroaromatic ring system which has 5 to 25 aromatic ring atoms and is substituted by R⁴ radicals, where Ar¹ and Ar² may optionally be joined together via a bond or an E group;
E is C(R⁴)₂, —C(R⁴)₂—C(R⁴)₂—, —C(R⁴)═C(R⁴)—, C═O, Si(R⁴)₂, NR⁴, O, S, S═O, or SO₂;
R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁵ radicals; and
where one or more CH₂ groups in the alkyl, alkoxy, alkenyl and alkynyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and
where one or more CH₂ groups in the alkyl, alkoxy, alkenyl and alkynyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C═C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; and the alkyl, alkoxy, alkenyl and alkynyl groups, aromatic ring systems and heteroaromatic ring systems mentioned may be substituted by one or more radicals selected from F and CN;
n is 0 or 1, where, when n=0, the G group and the nitrogen atom in formula (I) are bonded directly to one another.

What is meant in formula (G) by the group

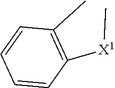

being shown as bonded in a nonspecific manner to the radical of the formula

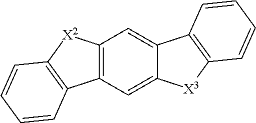

is that the two bonds labelled * below

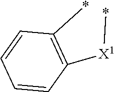

each emanate from a different carbon atom of the right-hand benzene ring in the remainder of the formula shown above, where the two carbon atoms to which the two bonds labelled * bind are adjacent to one another in the benzene ring. This makes X¹ part of a five-membered ring. No further restrictions are to be inferred from the above definition according to the application. In particular, it cannot be inferred from the fact that X¹ in formula (G) is shown as pointing “downward” that only embodiments of the type according to formulae (G-1), (G-3) and (G-5) shown below are encompassed. On the contrary, all six geometrically possible binding variants of the group

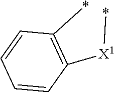

as shown representatively in the formulae (G-1) to (G-6) below are encompassed by the formula (G) of the present application. This definition is also applicable to all the subformulae of formula (G) and formula (I) that are shown below. It is apparent from this that formula (G) encompasses the following alternative embodiments:

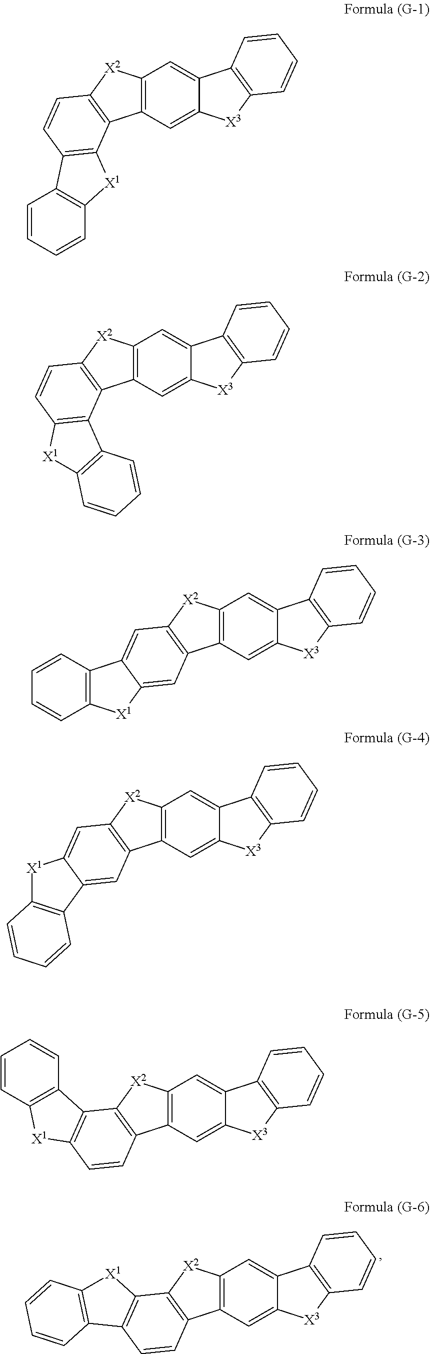

which are each defined otherwise like formula (G) above. Among the alternative embodiments of formula (G) mentioned, preference is given to formula (G-1).

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be substituted by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

In a preferred embodiment, X¹ is O.

In a preferred embodiment, X² and X³ are each C(R²)₂.

In a particularly preferred embodiment, X¹ is O, and X² and X³ are each C(R²)₂.

Ar^{L} is preferably selected from aromatic ring systems which have 6 to 25 aromatic ring atoms and are substituted by R³ radicals, and is more preferably selected from phenyl, biphenyl, naphthyl or fluorenyl, each substituted by R³ radicals, and is most preferably selected from phenyl substituted by R³ radicals.

Preferably, Ar^{L} is the same or different at each instance and is selected from groups of the following formulae:

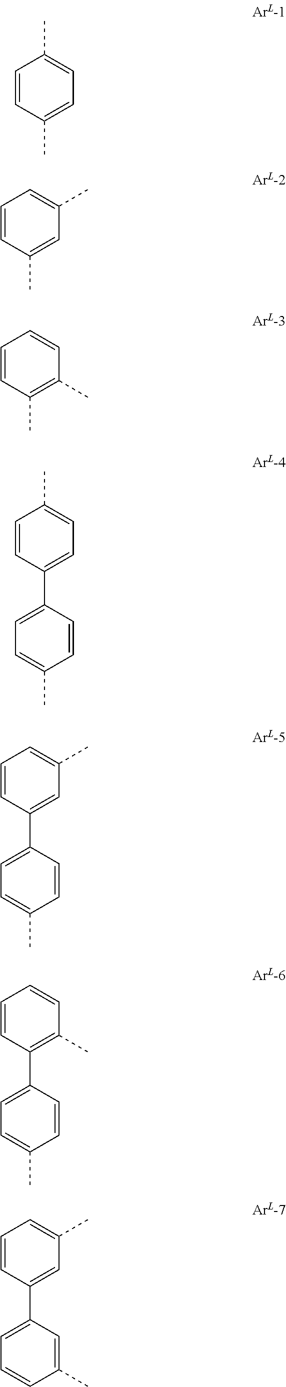

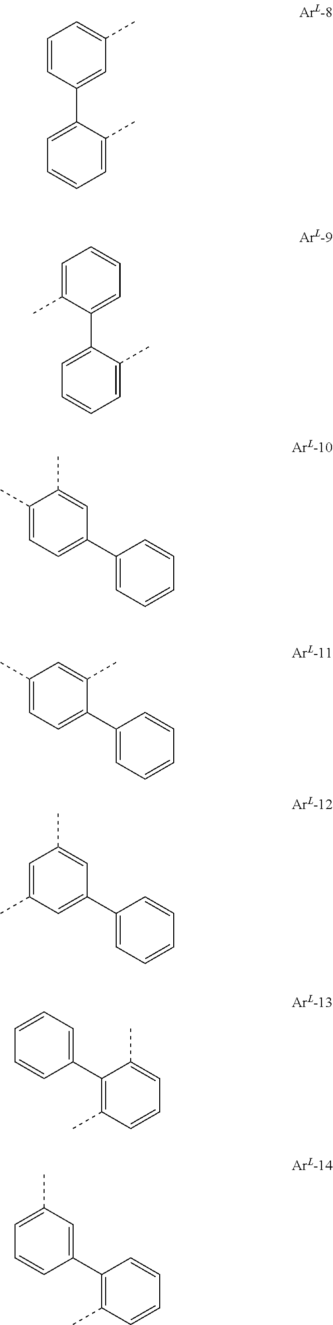

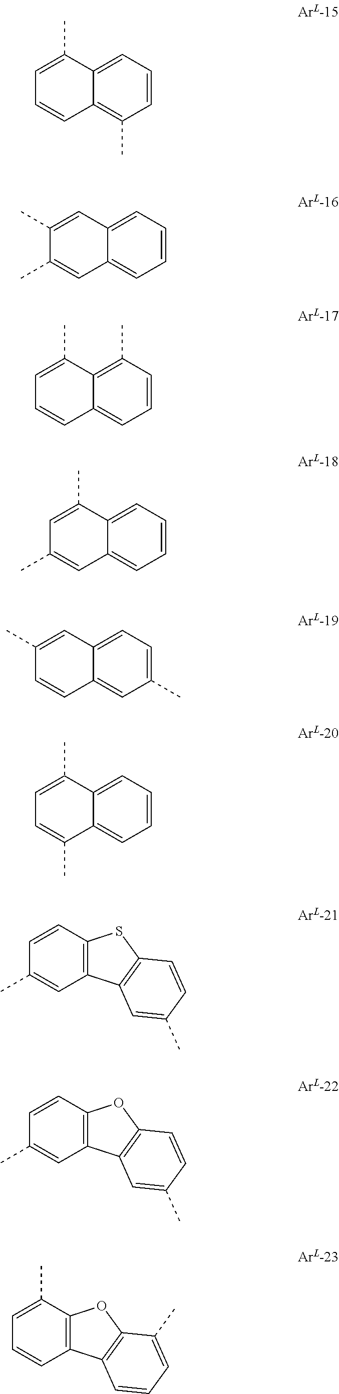

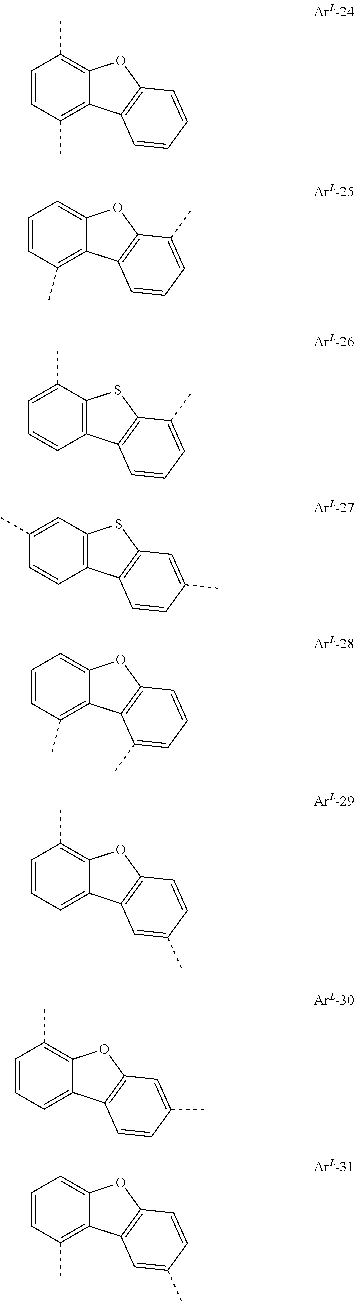

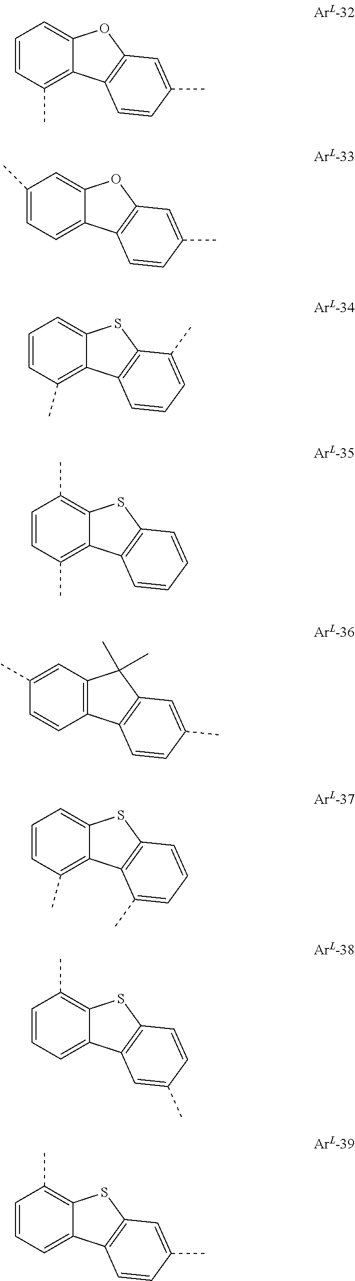

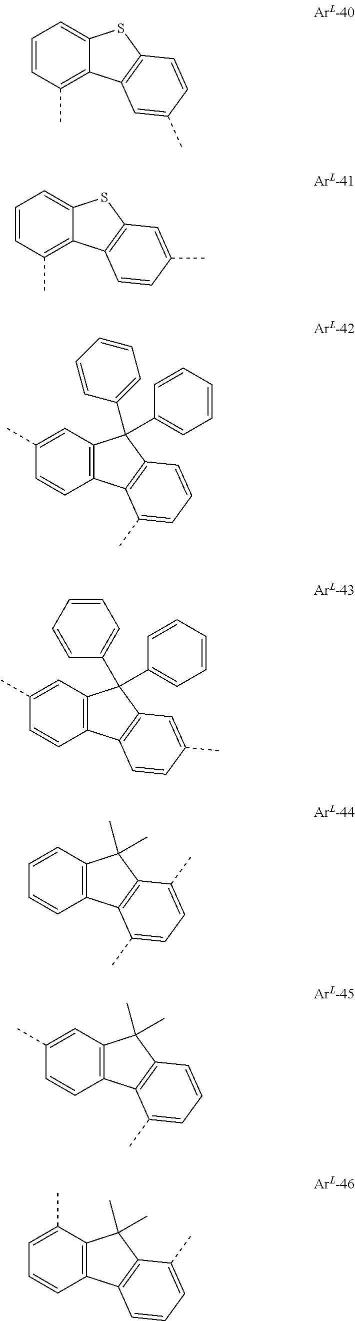

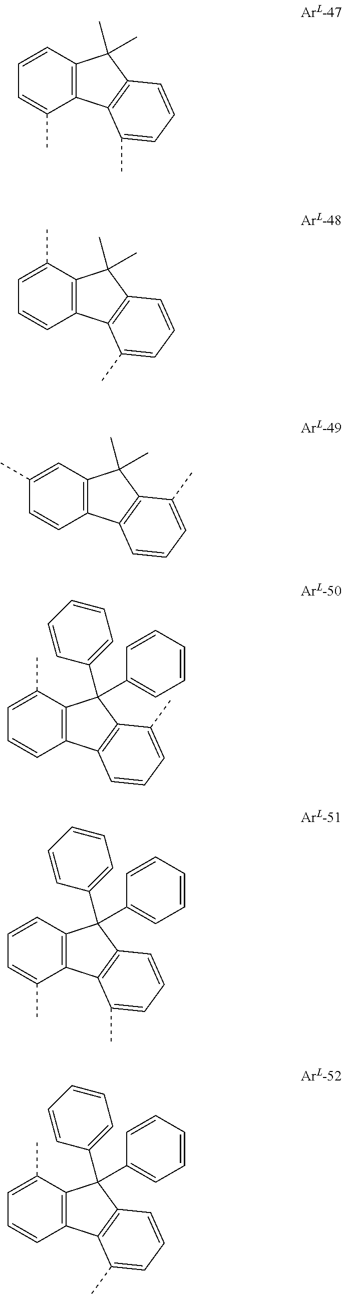

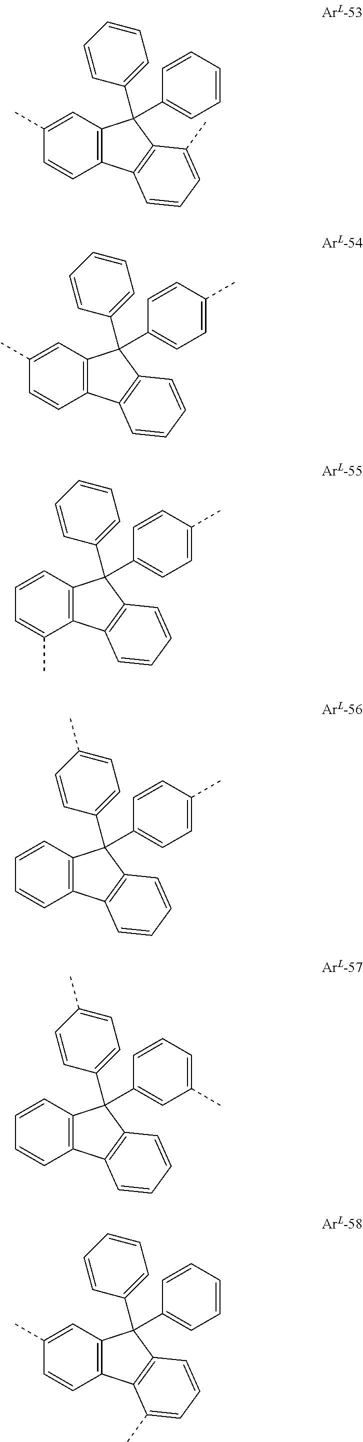

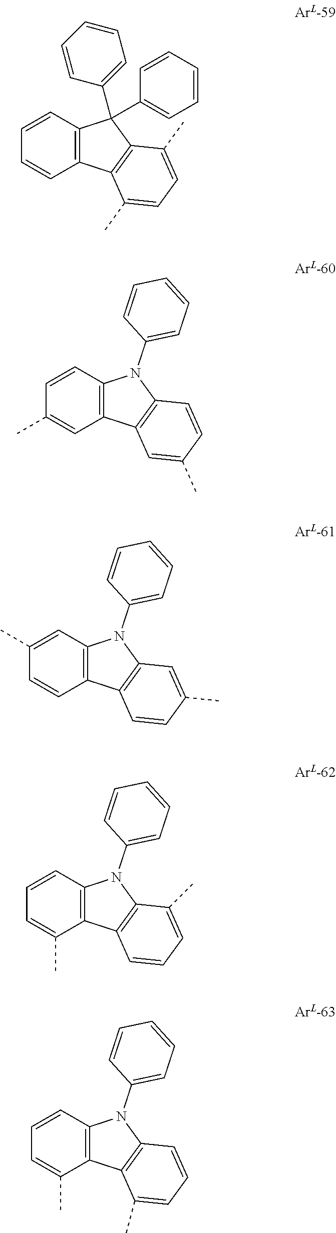

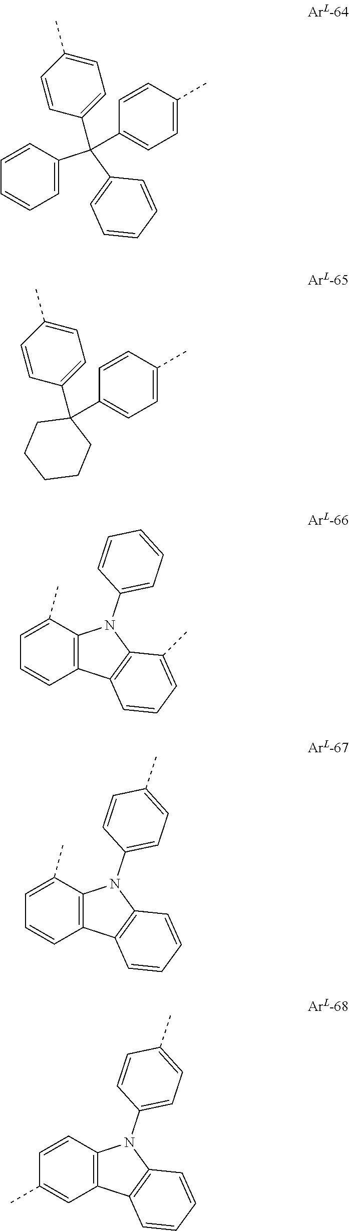

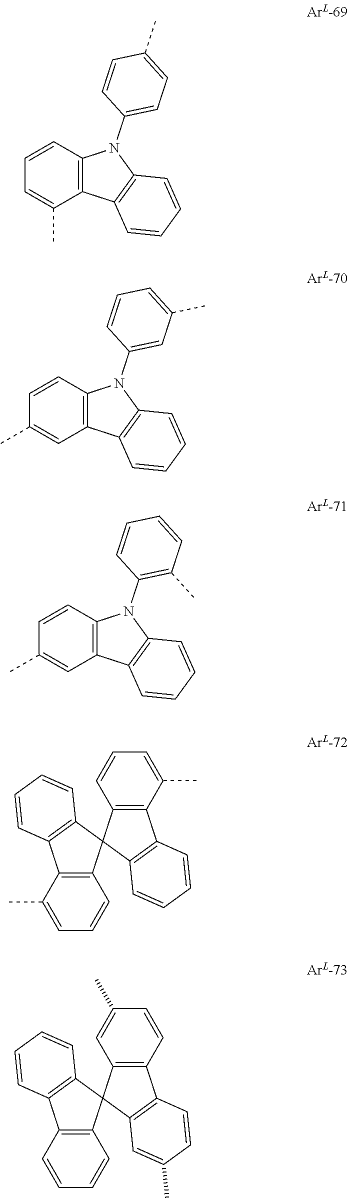

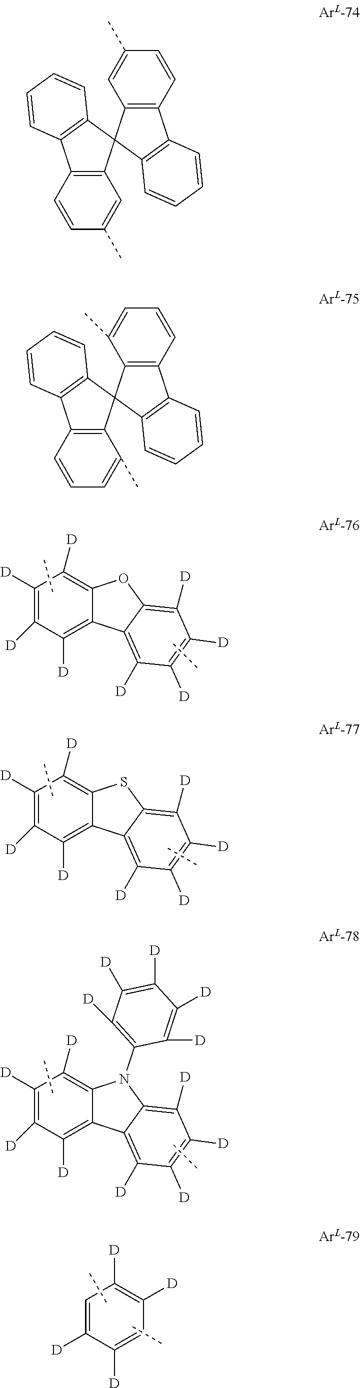

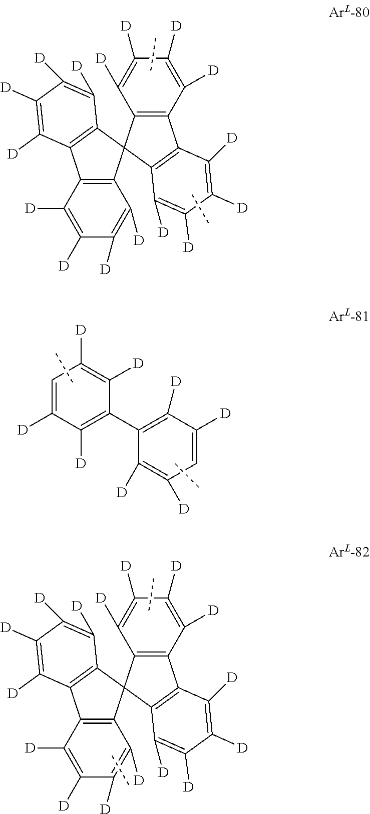

where the dotted lines represent the bonds to the rest of the formula.

In a preferred embodiment, the index n=0. In an alternative preferred embodiment, the index n=1.

Ar¹ and Ar² are the same or different at each instance and are selected from aromatic ring systems which have 6 to 25 aromatic ring atoms and are substituted by R⁴ radicals, and heteroaromatic ring systems which have 5 to 25 aromatic ring atoms and are substituted by R⁴ radicals.

Ar¹ and Ar² are preferably the same or different and are selected from the following formulae:

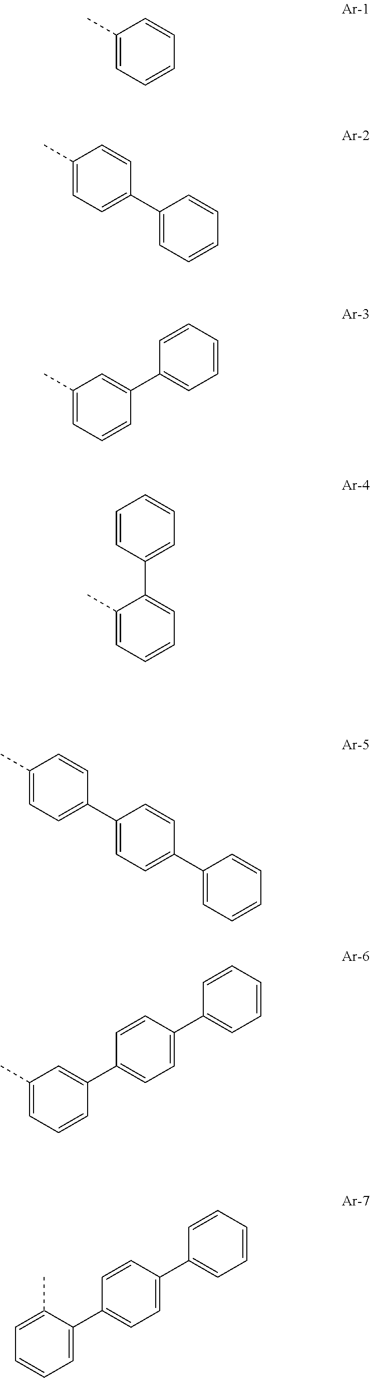

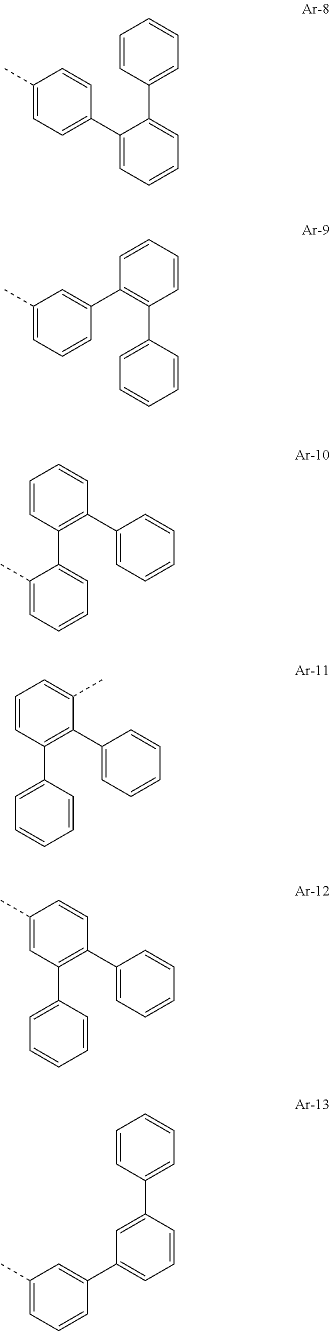

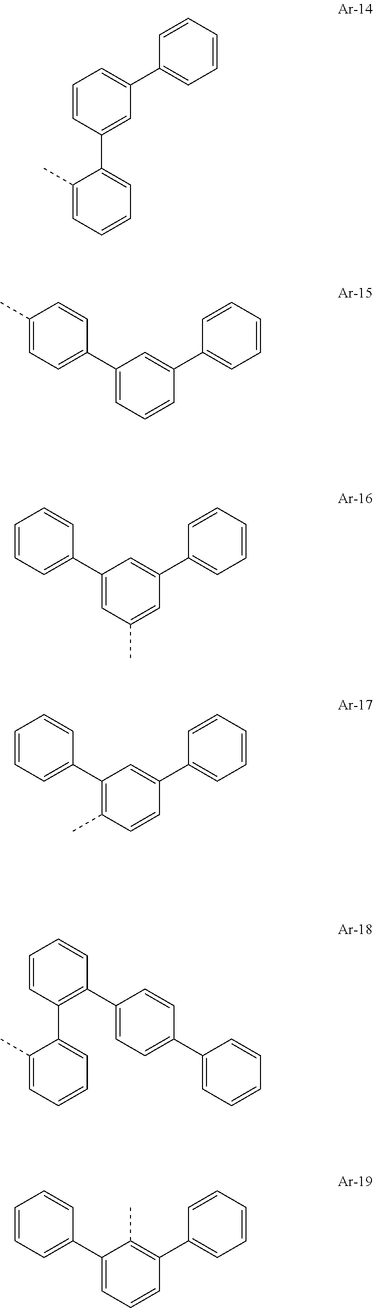

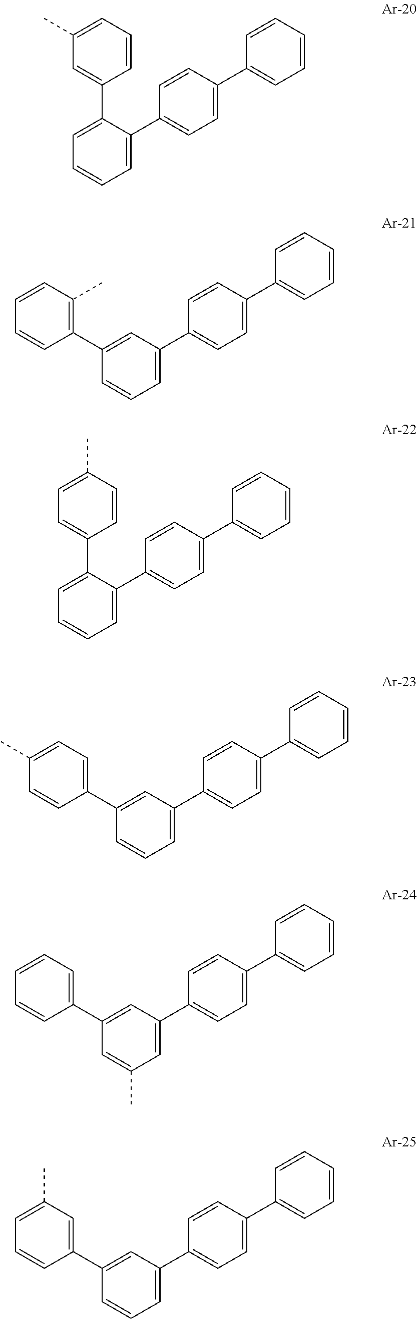

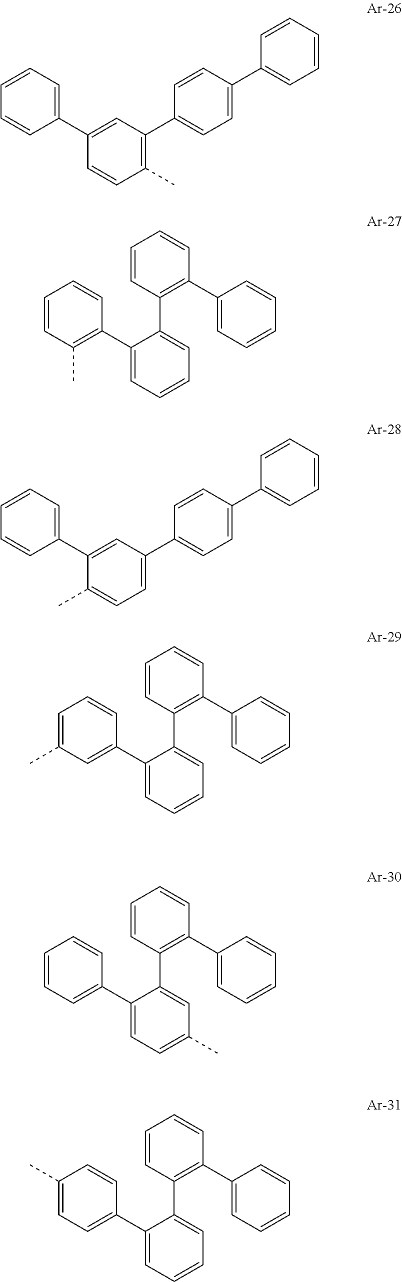

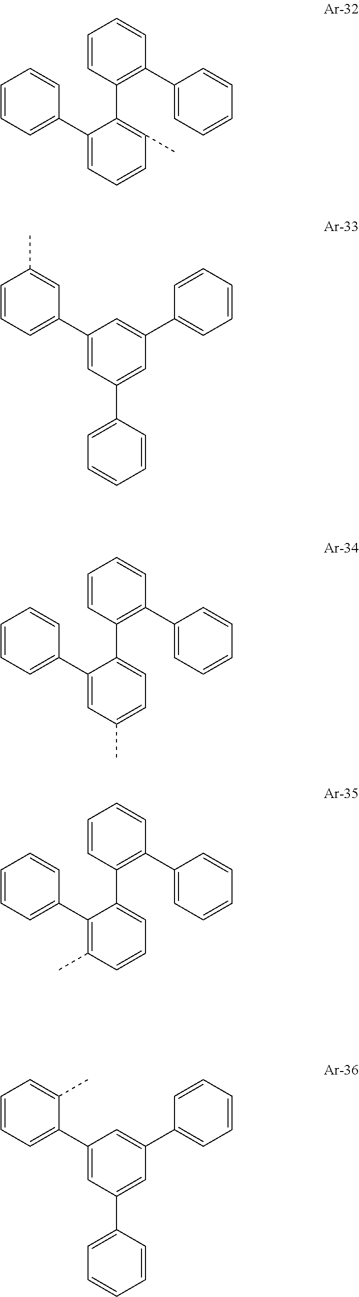

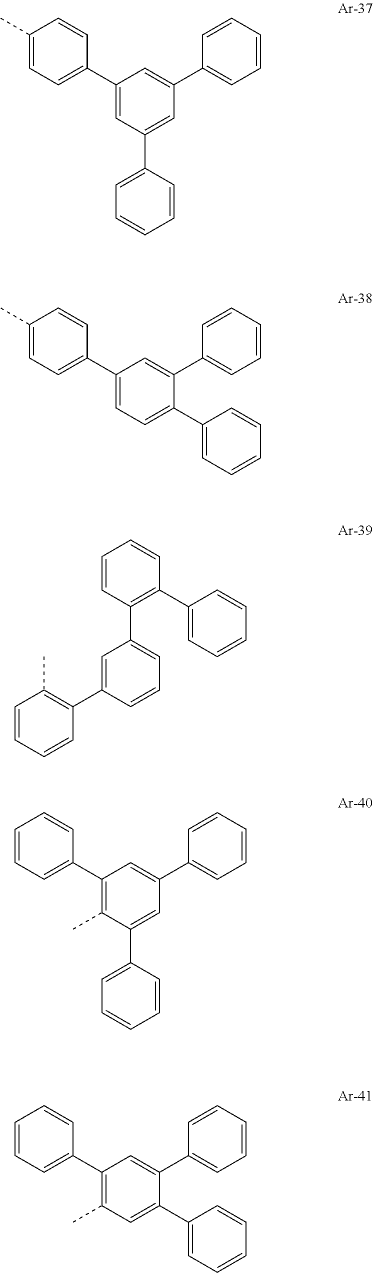

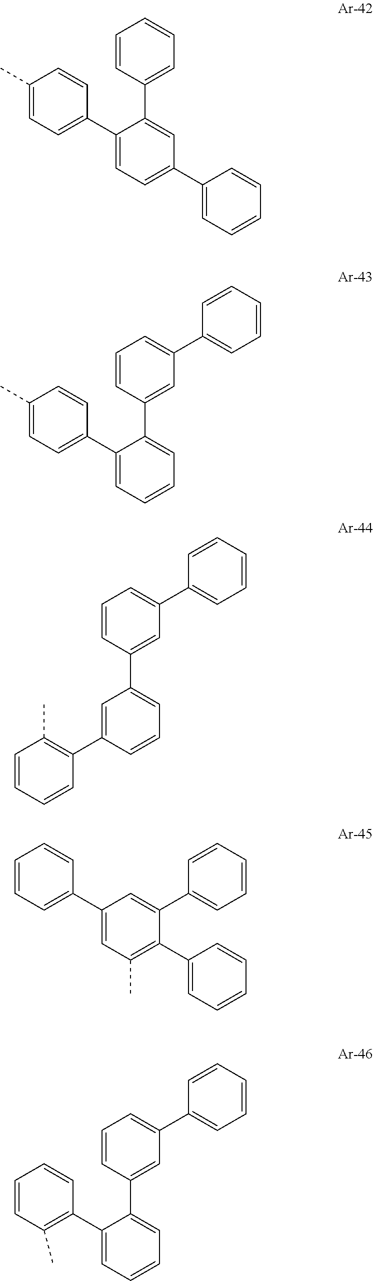

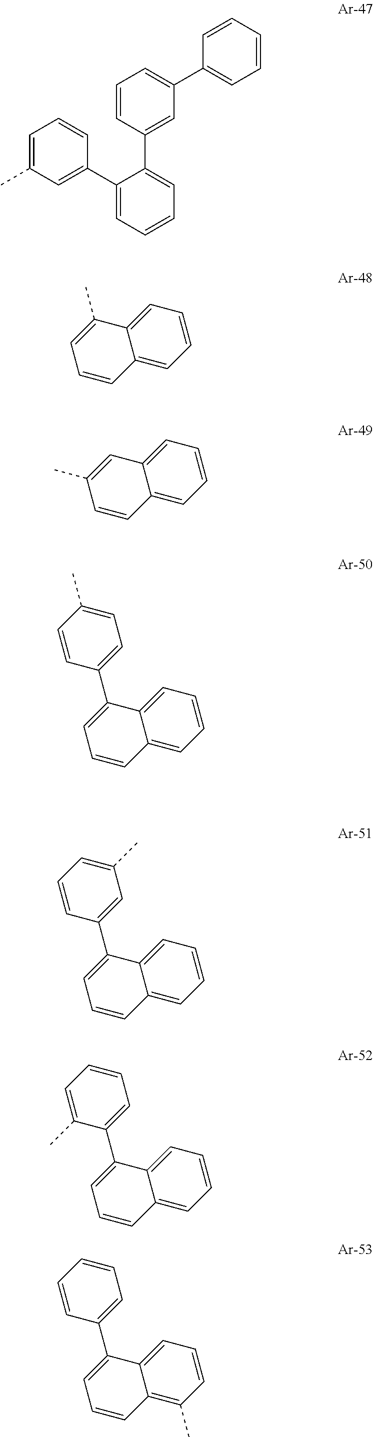

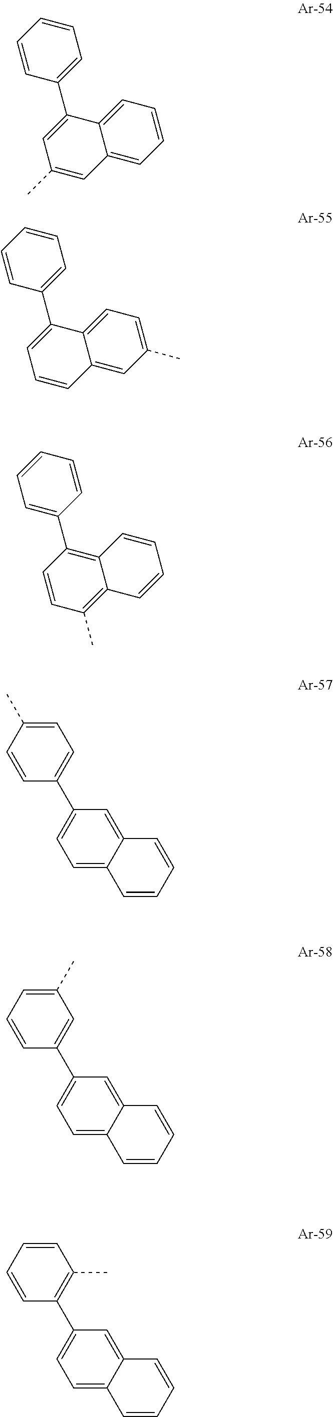

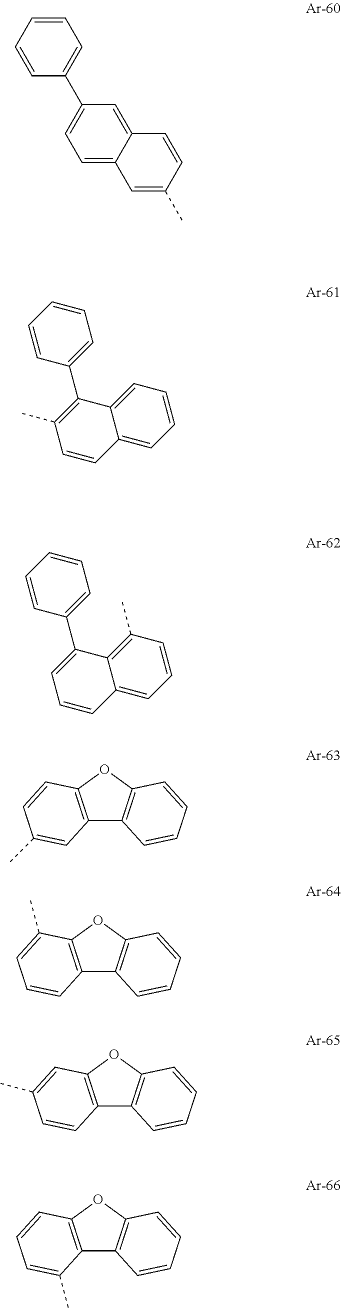

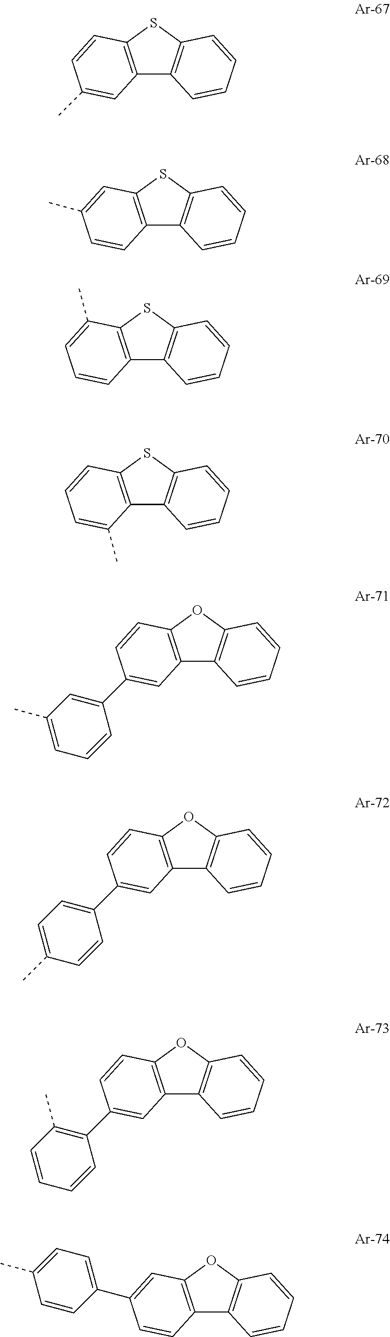

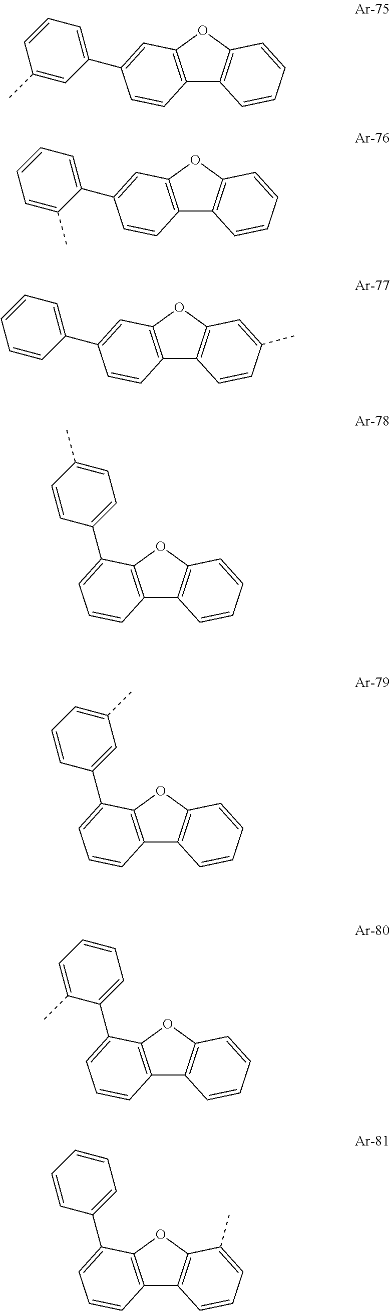

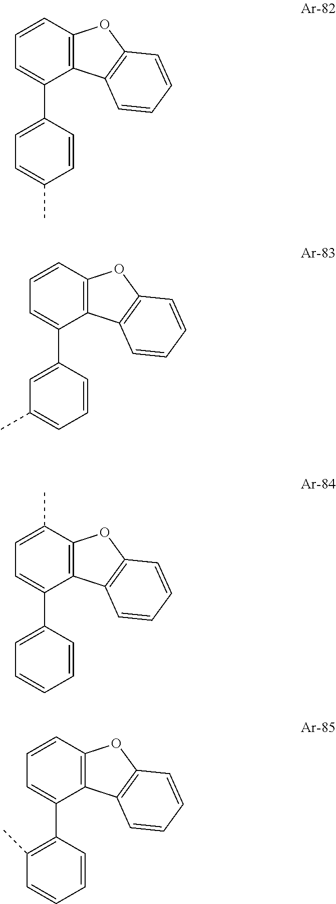

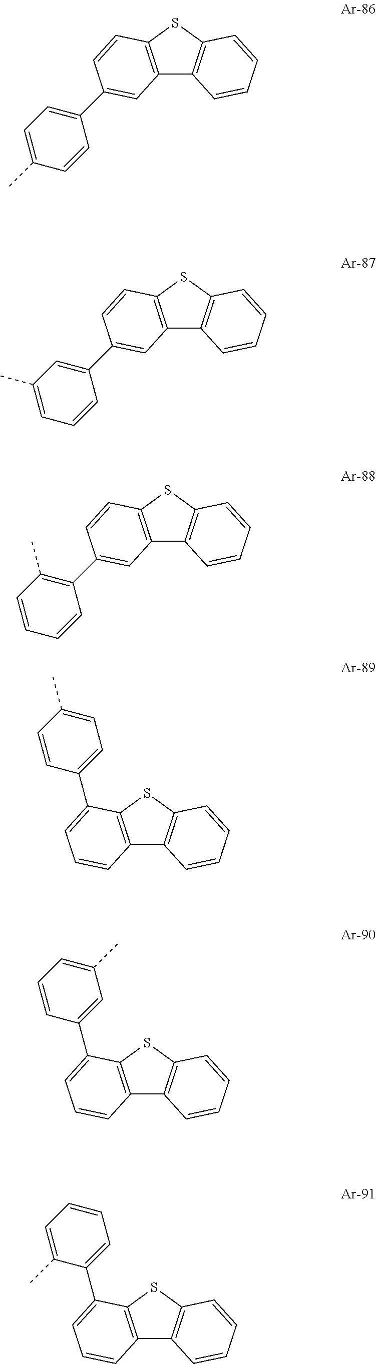

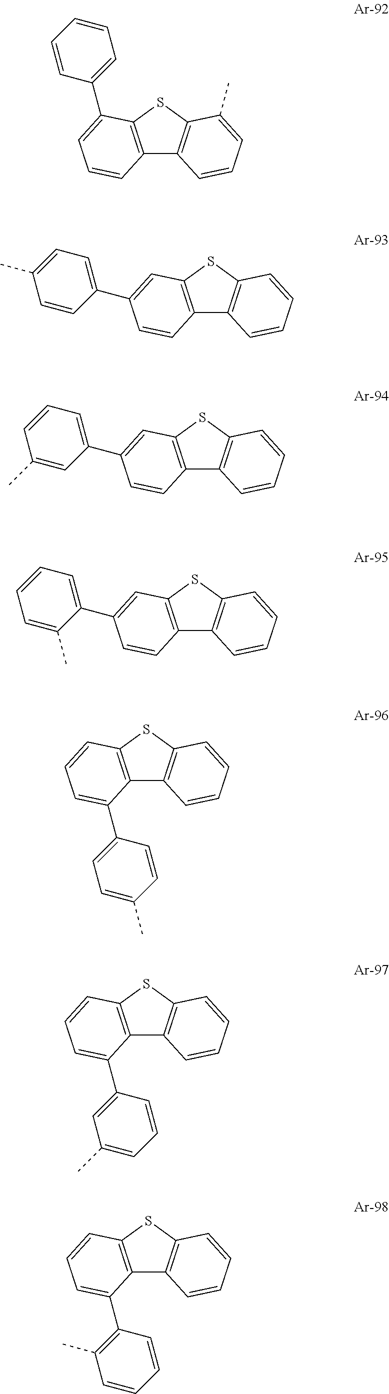

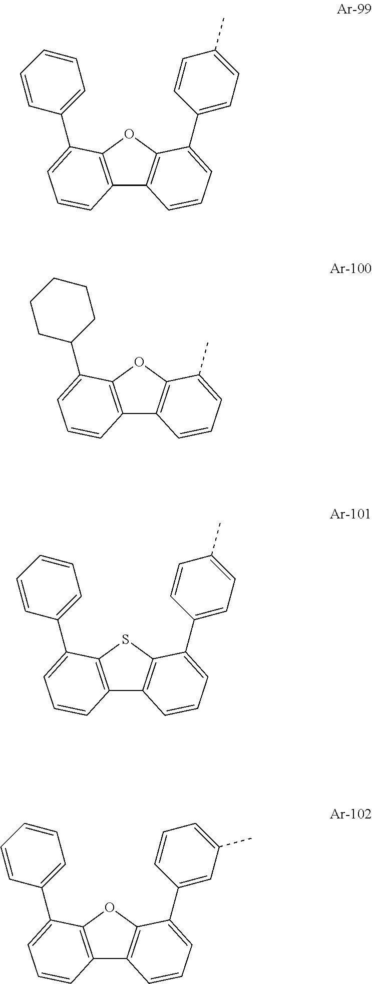

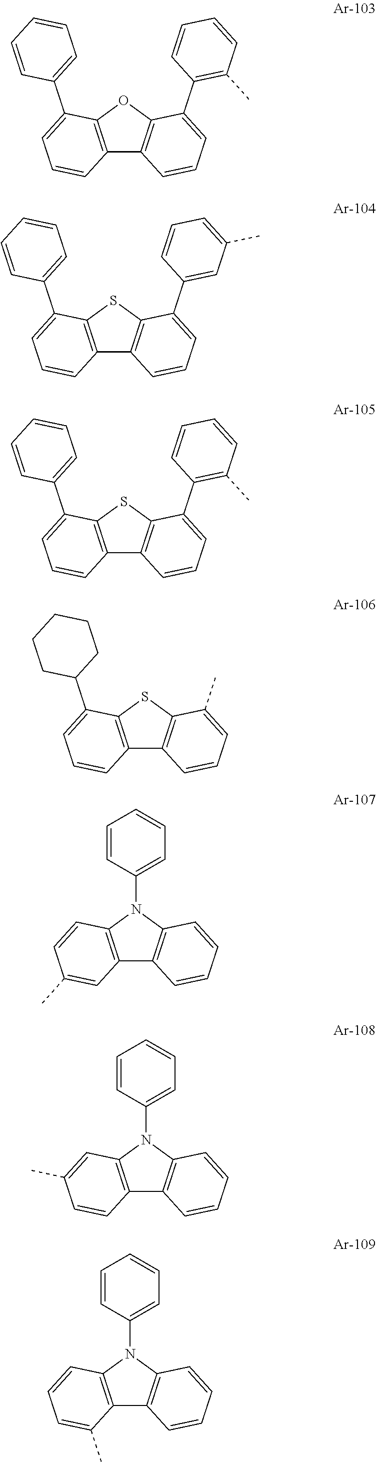

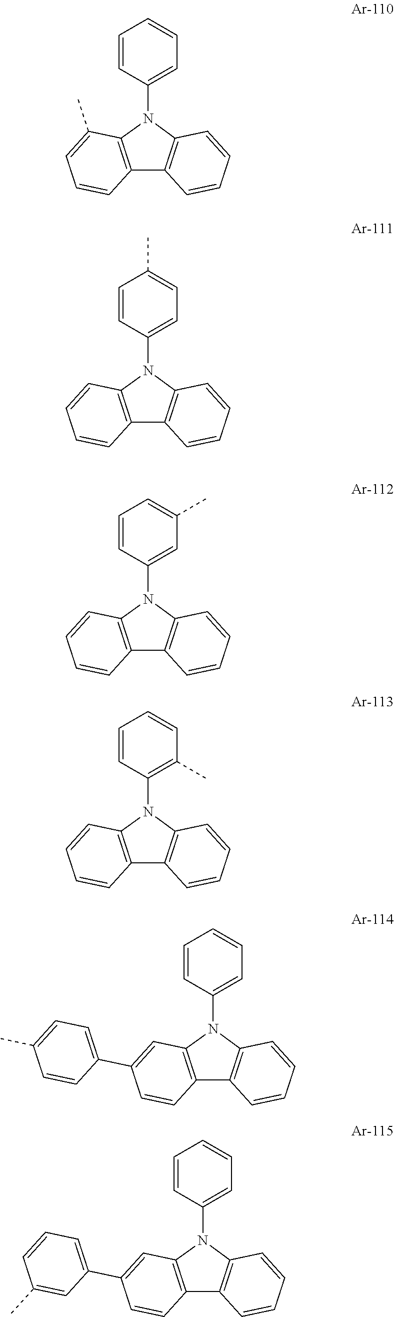

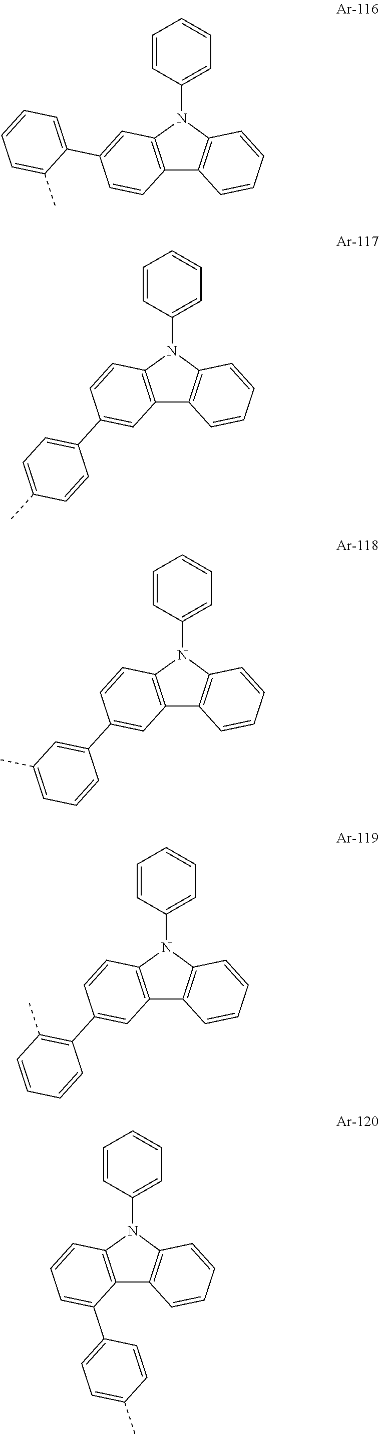

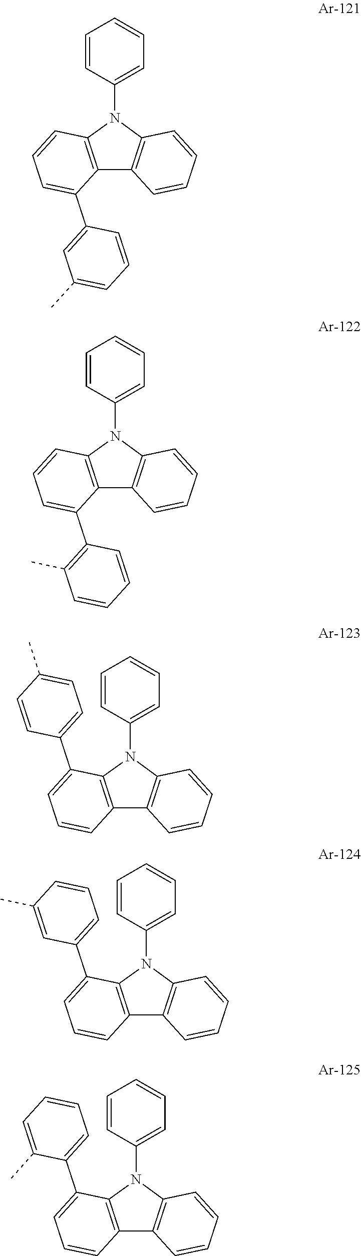

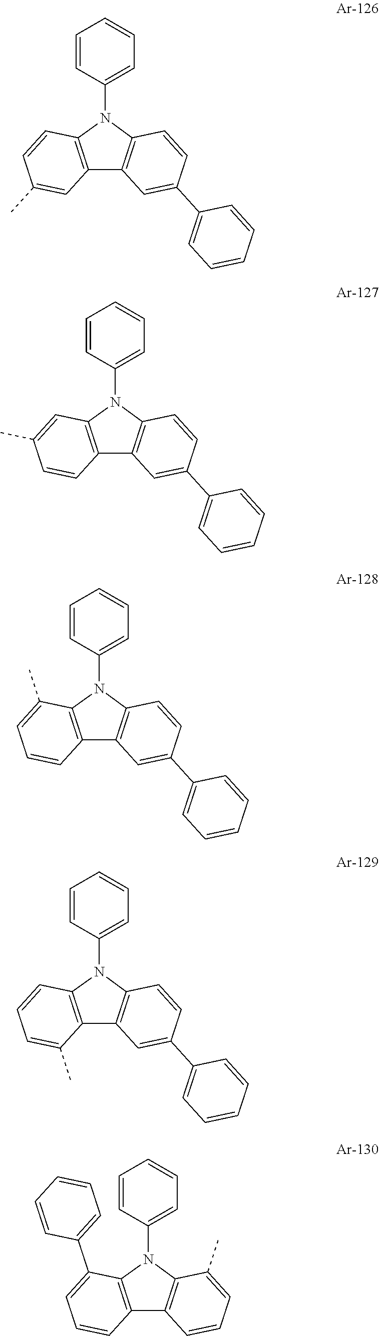

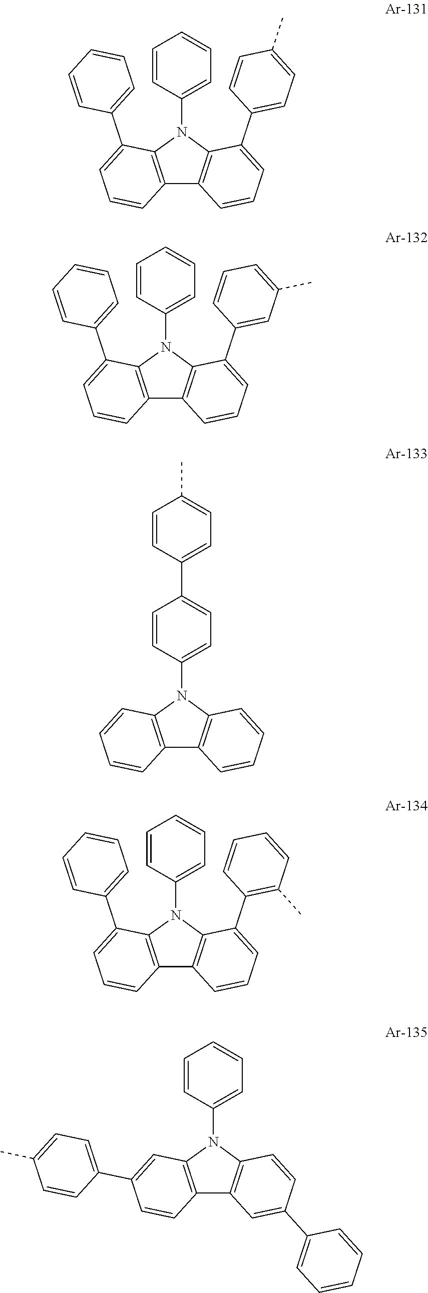

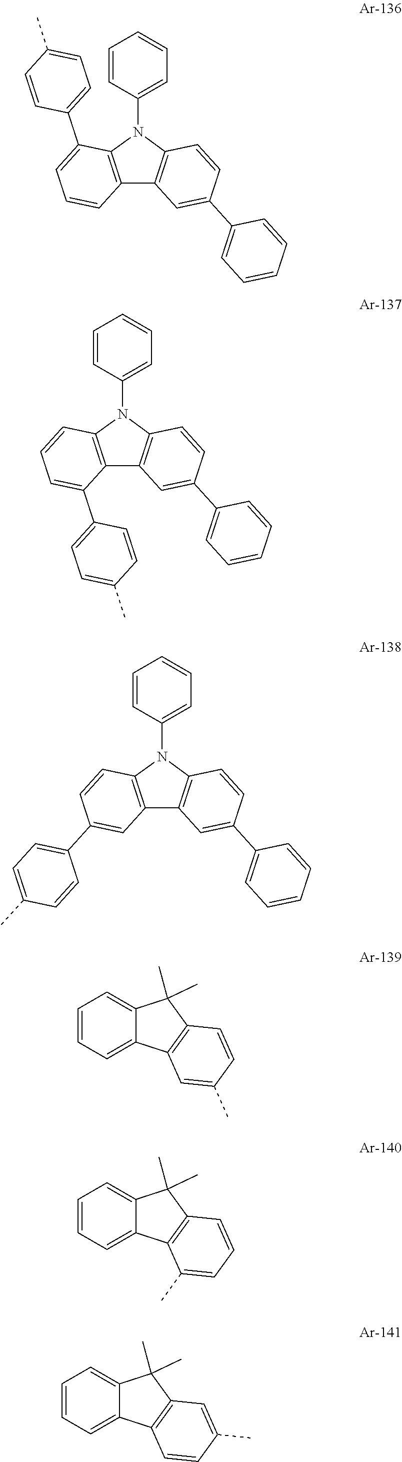

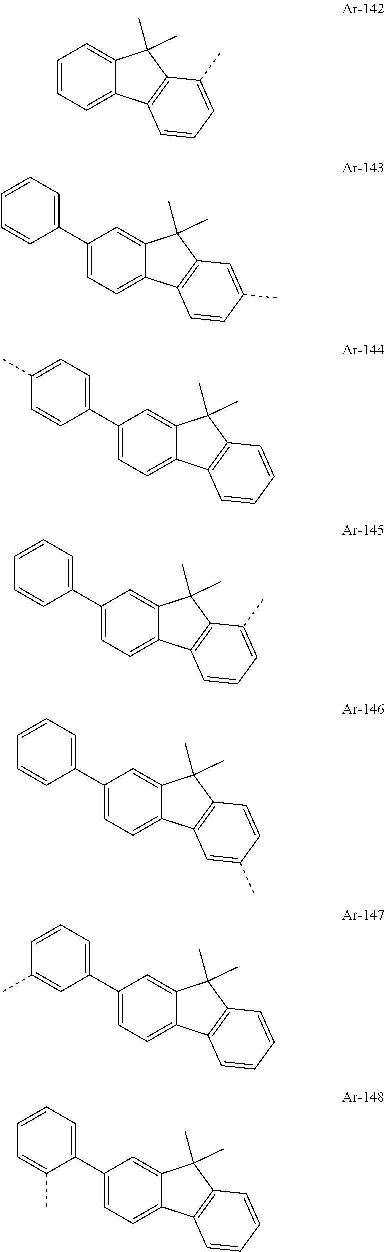

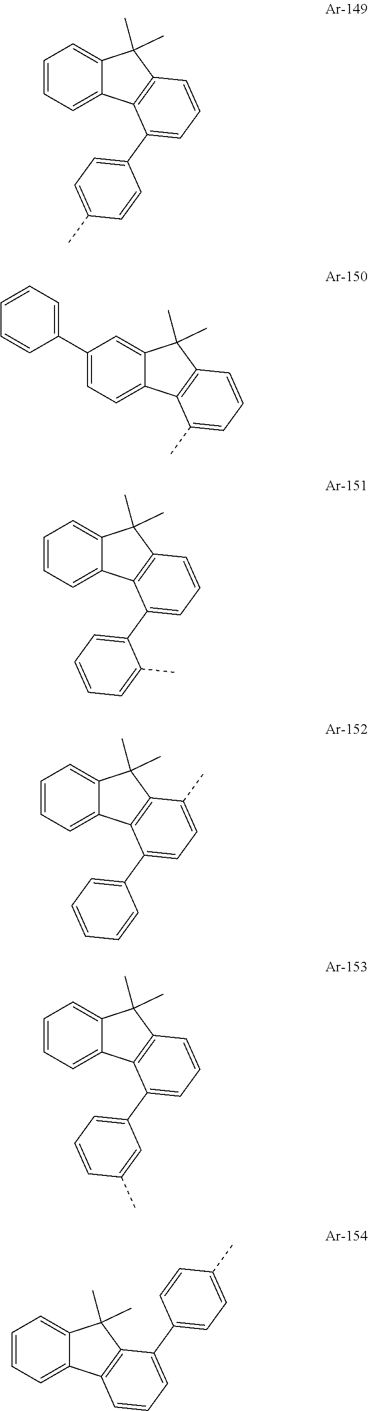

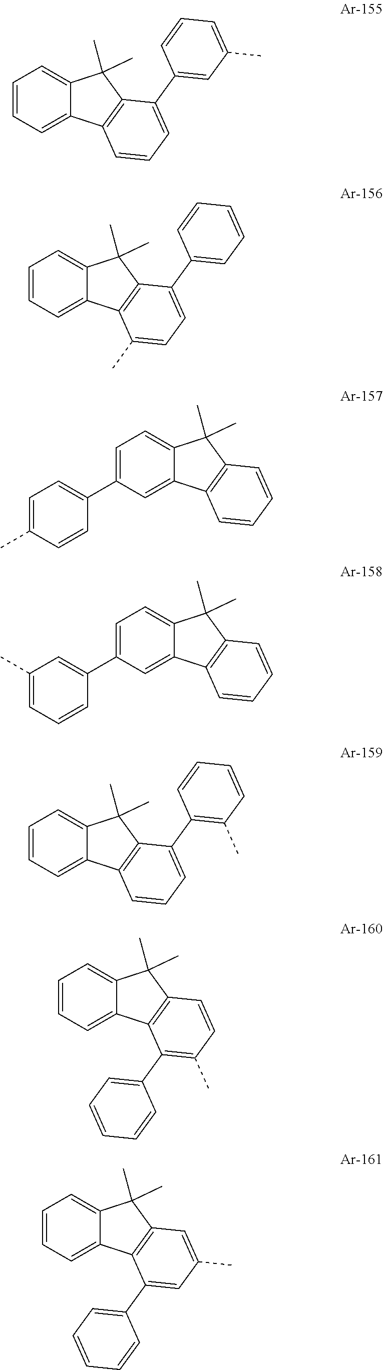

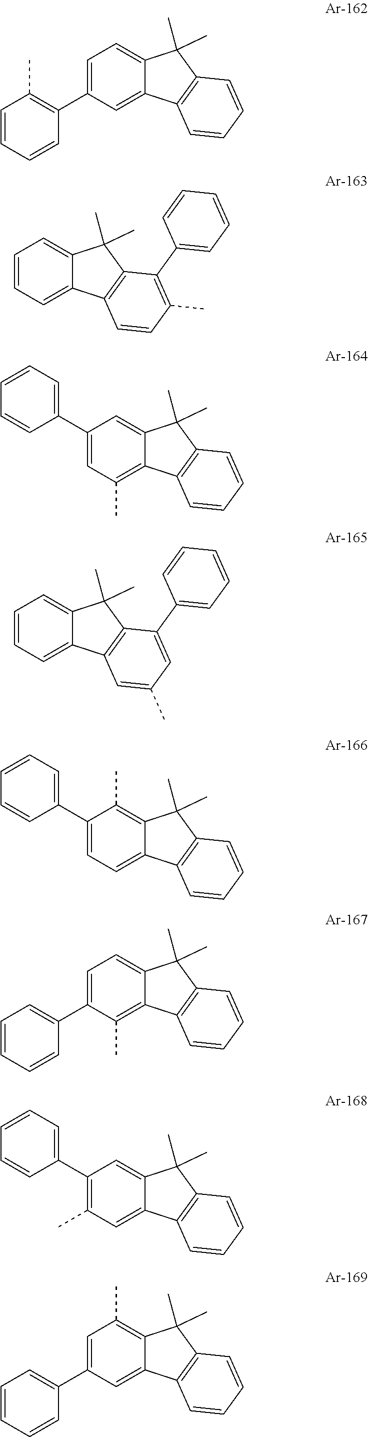

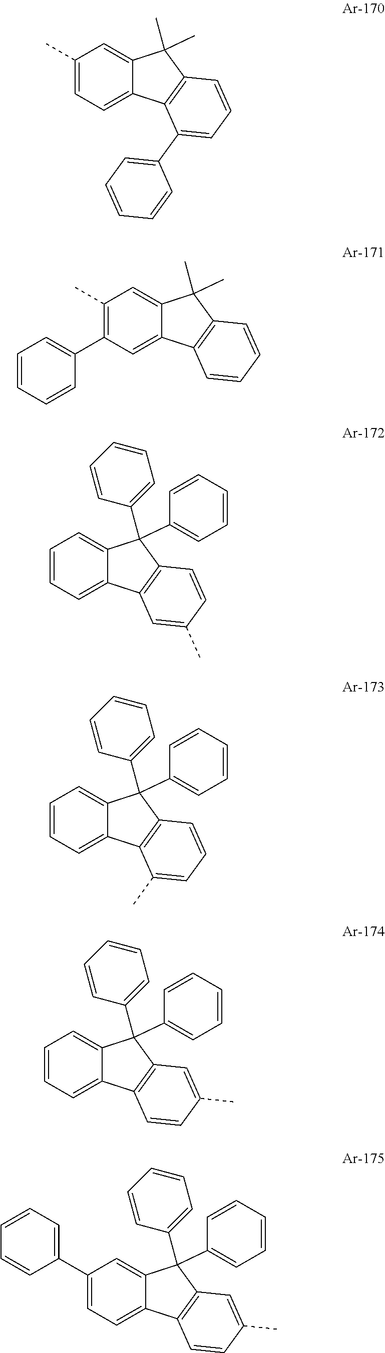

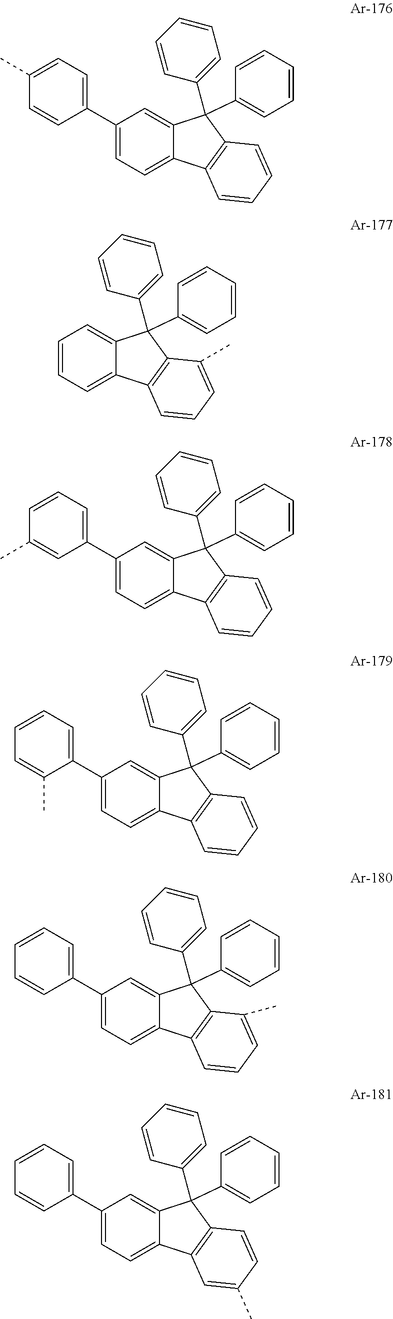

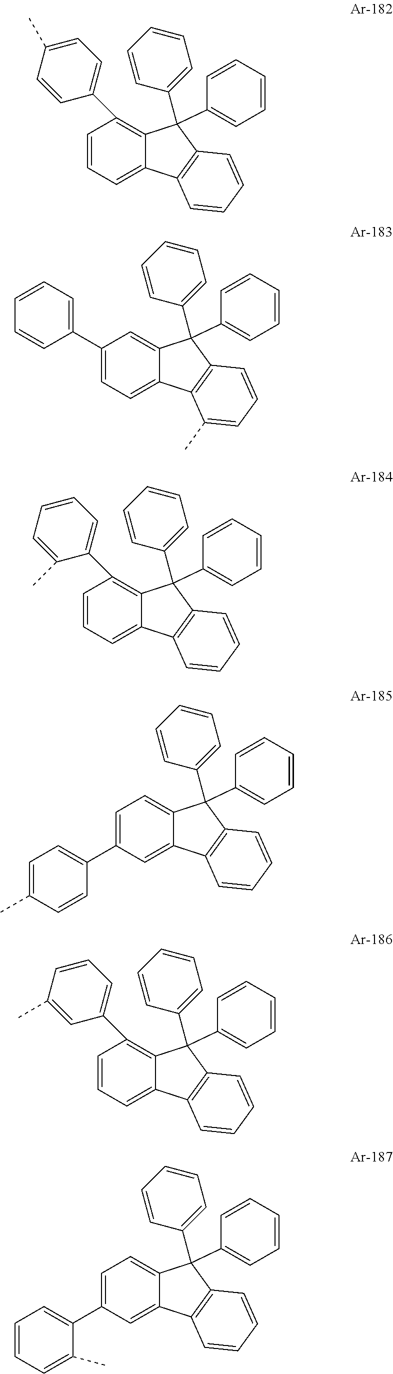

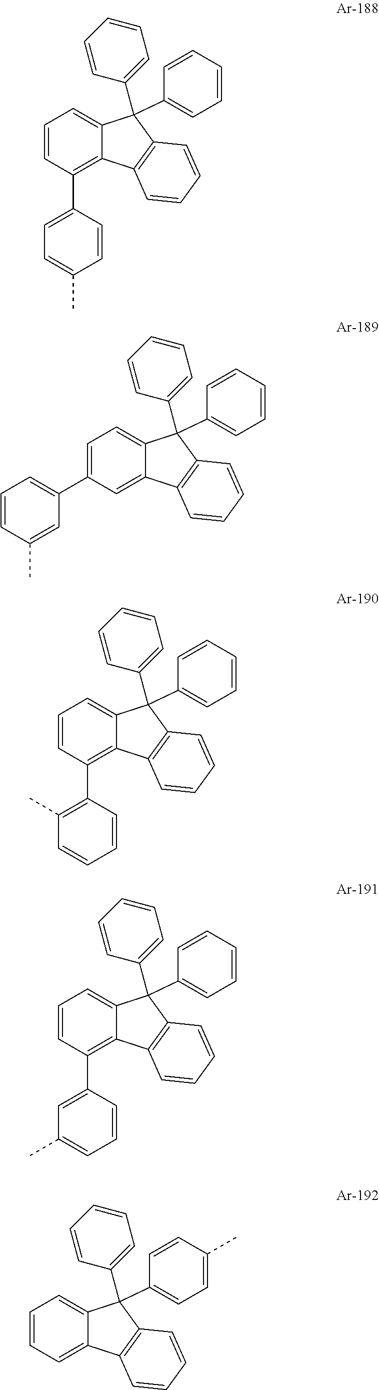

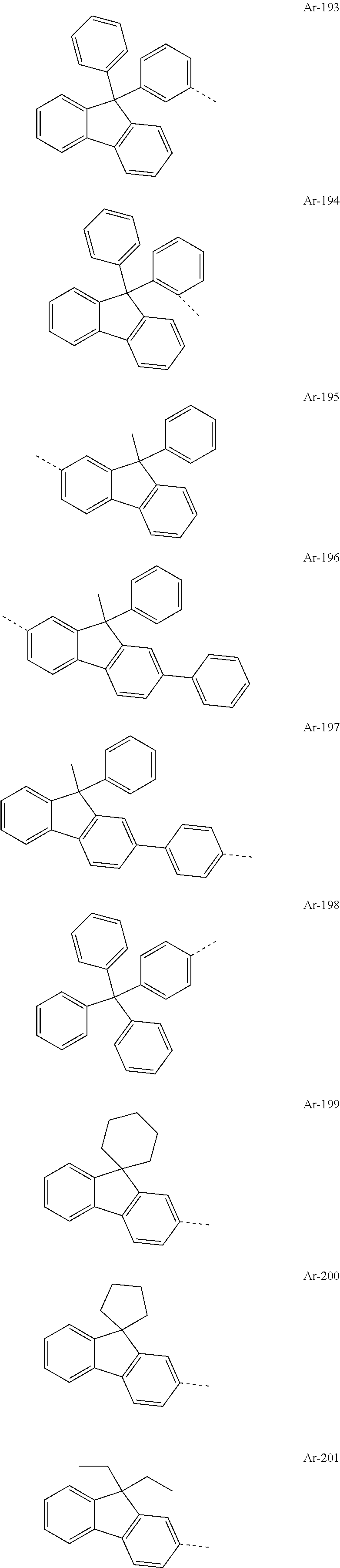

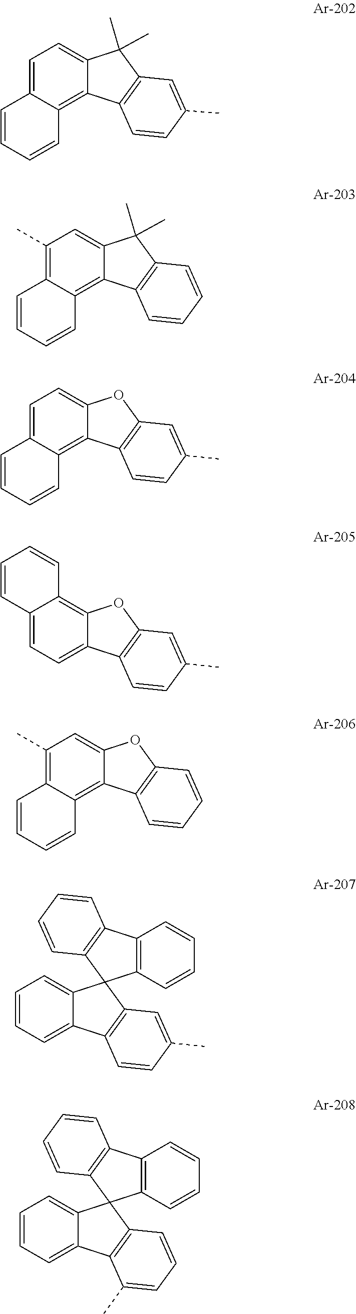

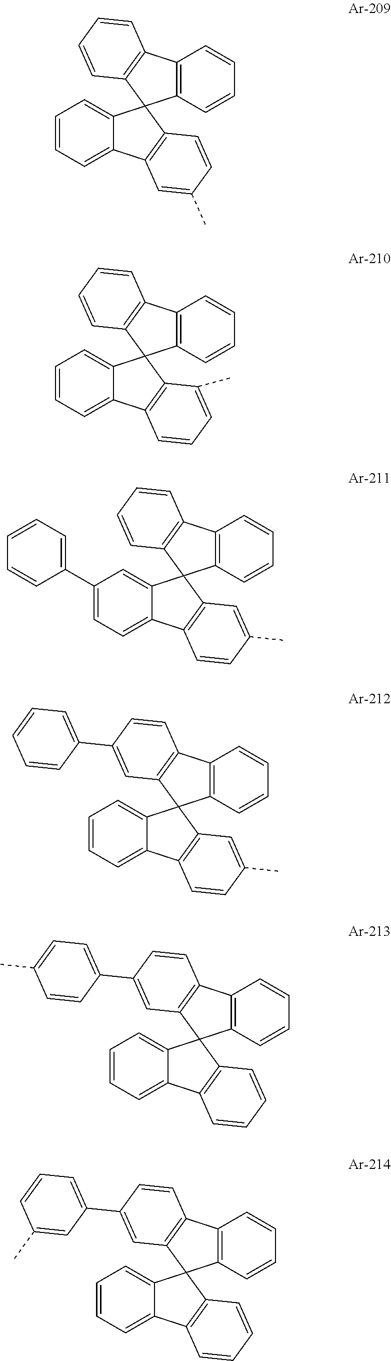

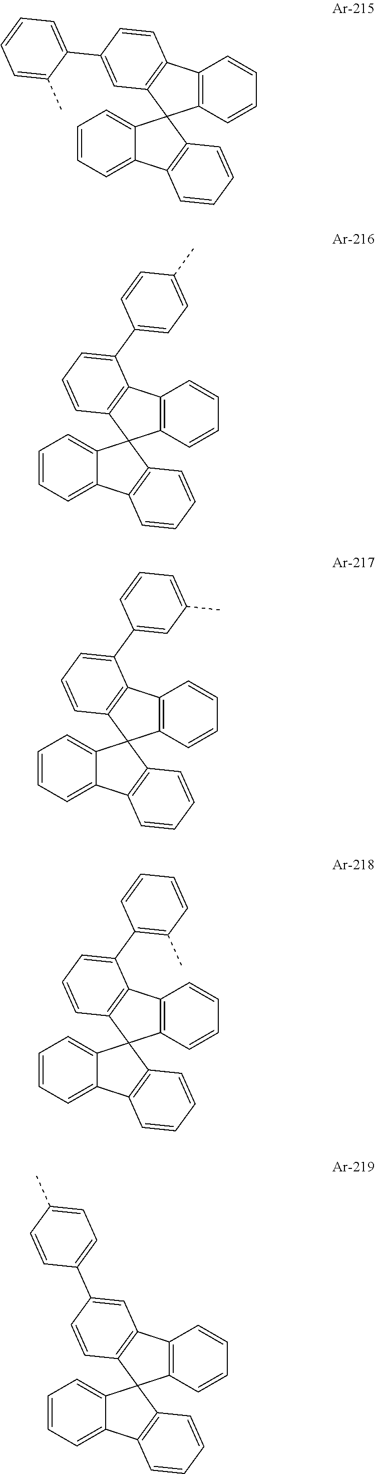

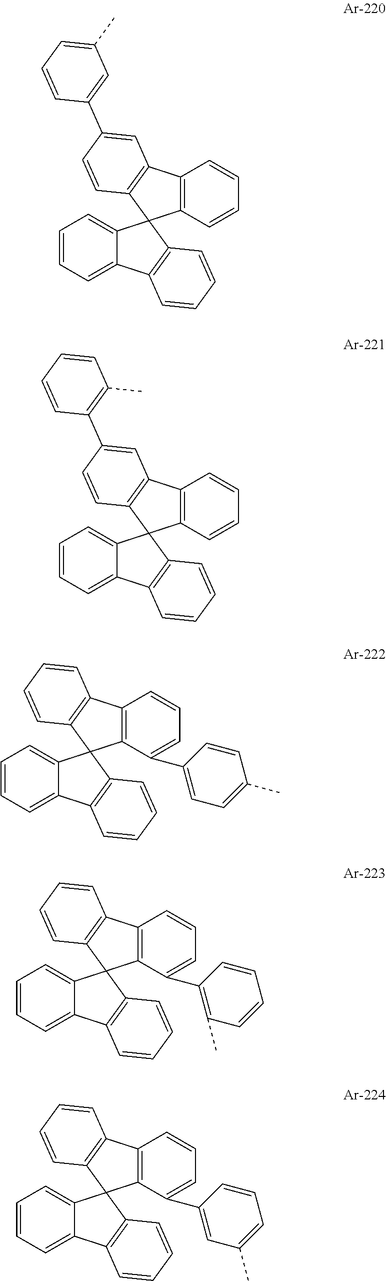

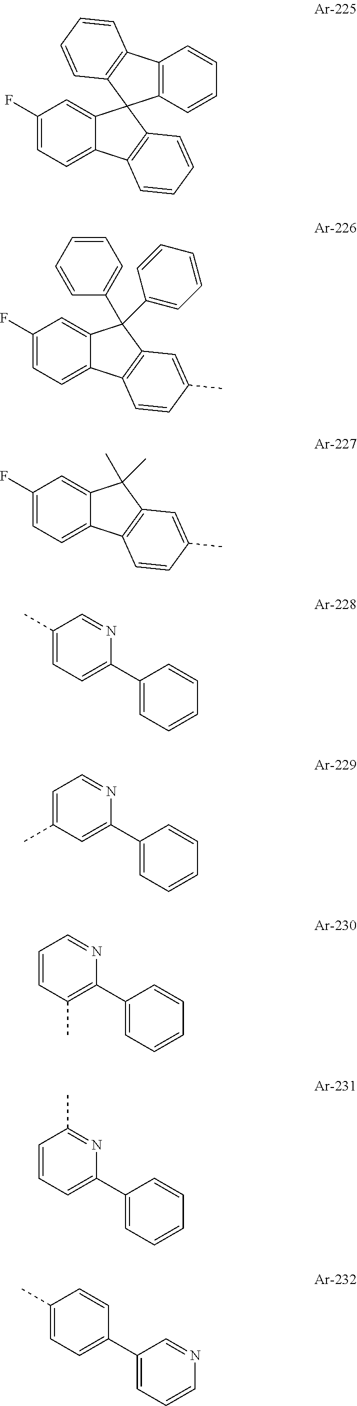

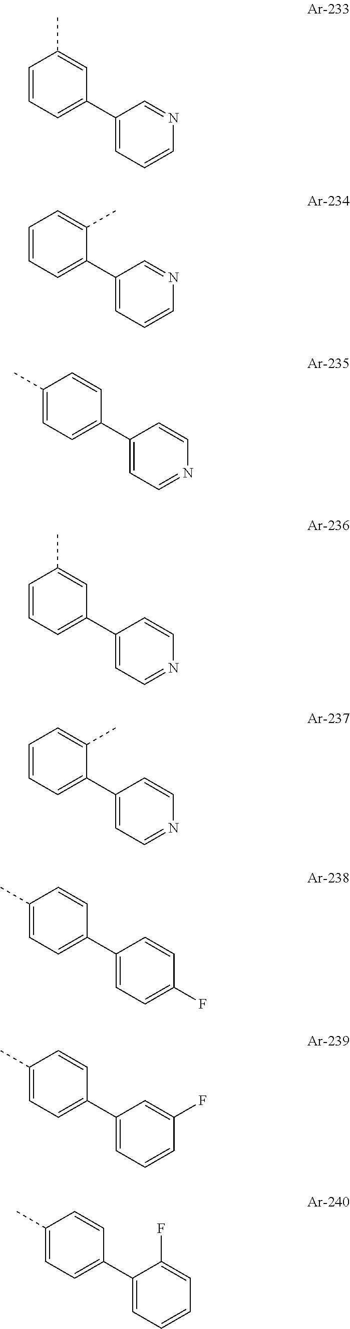

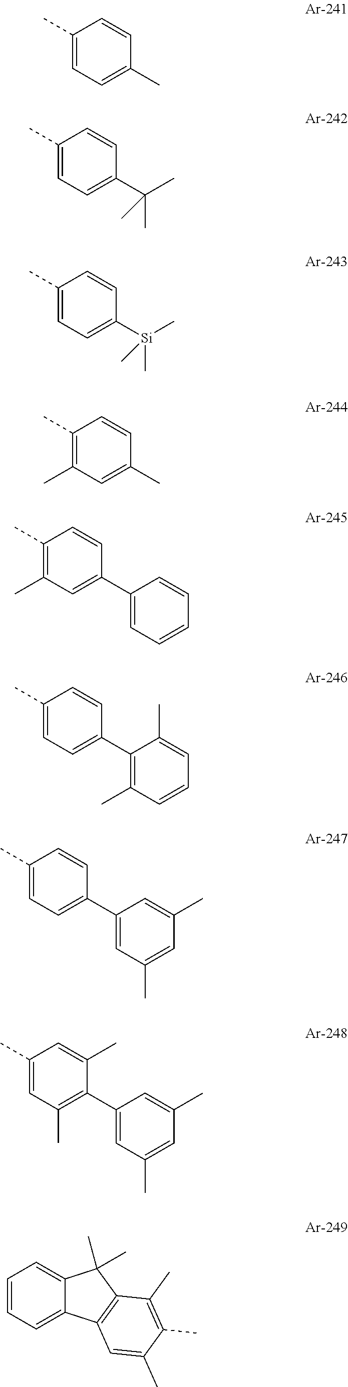

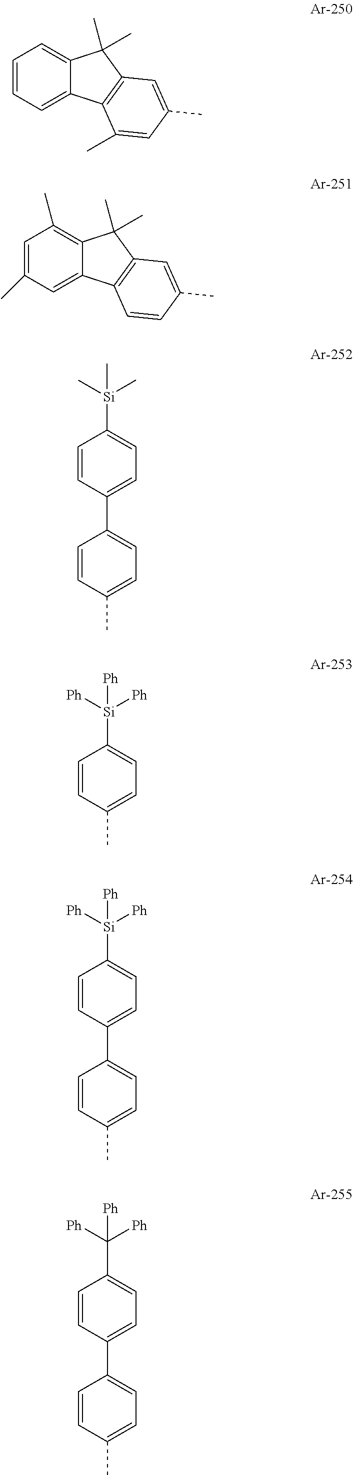

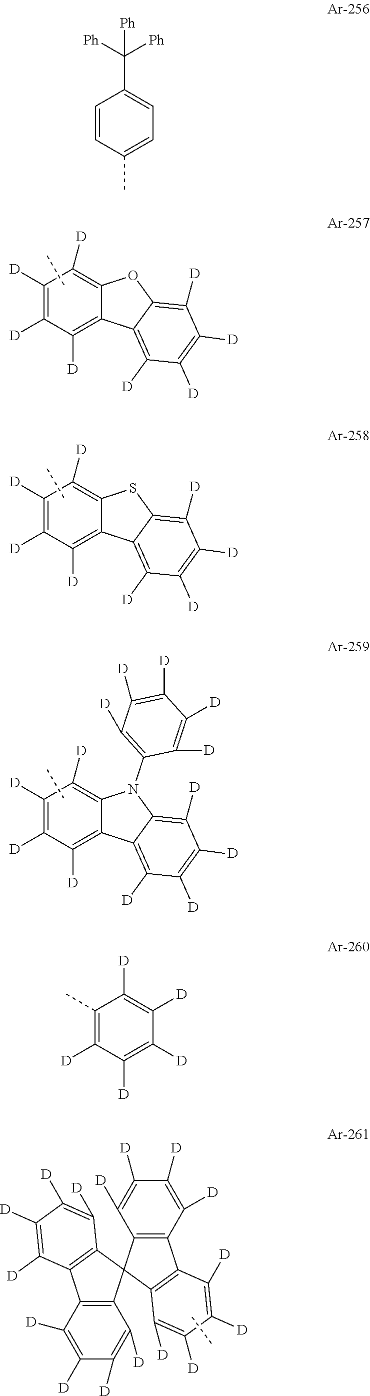

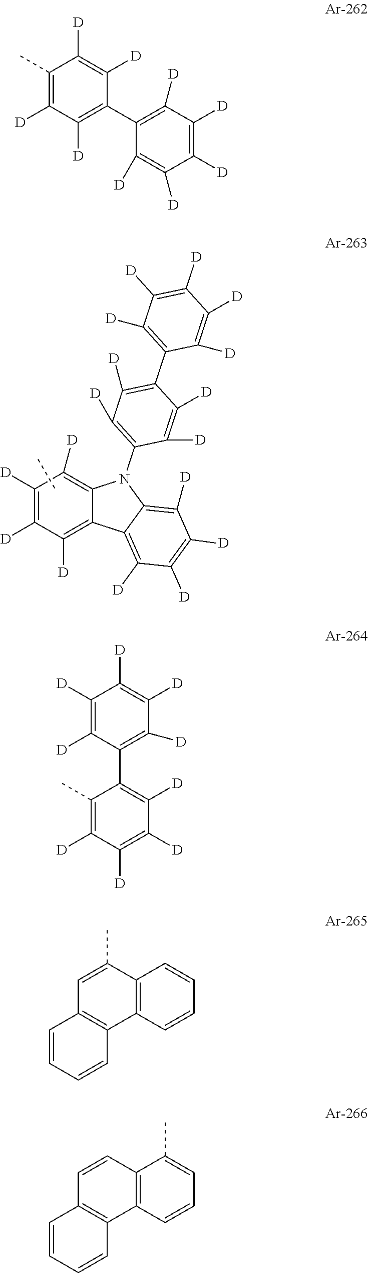

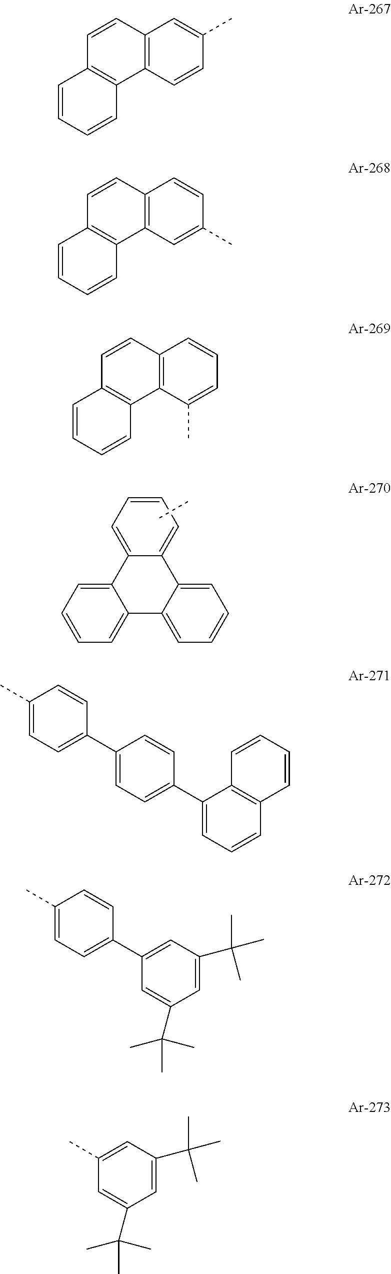

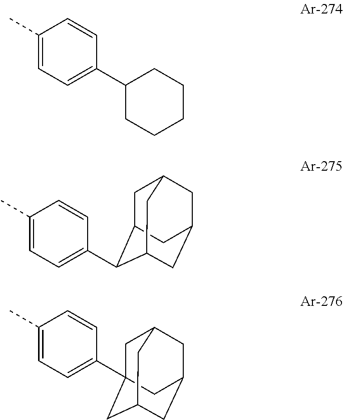

where the dotted line represents the bond to the nitrogen atom and where the groups at the position is shown as unsubstituted may be substituted by R⁴ radicals, and preferably have only H in the positions shown as unsubstituted. Some of the Ar-1 to Ar-276 groups shown also contain one or more R⁴ radicals.

It is preferable that at least one of the Ar¹ and Ar² groups is an aromatic ring system which has at least 12 aromatic ring atoms and is substituted by R⁴ radicals.

In a preferred embodiment, Ar¹ and Ar² are not bonded to one another via a bond or an E group.

R¹ is preferably the same or different at each instance and is selected from H, D, F, CN, Si(R⁵)₃, N(R⁵)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where the alkyl groups mentioned, the aromatic ring systems mentioned and the heteroaromatic ring systems mentioned are each substituted by R⁵ radicals; and where one or more CH₂ groups in the alkyl groups mentioned may be replaced by —C═C—, —R⁵C═CR⁵—, Si(R⁵)₂, C═O, C═NR⁵, —NR⁵—, —O—, —S—, —C(═O)O— or —C(═O)NR⁵—. More preferably, R¹ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals. Most preferably, R¹ is H.

R² is preferably the same or different at each instance and is selected from straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl groups mentioned, the aromatic ring systems mentioned and the heteroaromatic ring systems mentioned are each substituted by R⁵ radicals; and where one or more CH₂ groups in the alkyl groups mentioned may be replaced by —C═C—, —R⁵C═CR⁵—, Si(R⁵)₂, C═O, C═NR⁵, —NR⁵—, —O—, —S—, —C(═O)O— or —C(═O)NR⁵—. More preferably, R² is the same or different at each instance and is selected from straight-chain alkyl groups having 1 to 20 carbon atoms and branched or cyclic alkyl groups having 3 to 20 carbon atoms; where two or more R² radicals may be joined to one another and may form a ring; where the alkyl groups mentioned may each be substituted by R⁵ radicals; and where one or more CH₂ groups in the alkyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂. Even more preferably, R² is the same or different at each instance and is selected from straight-chain alkyl groups having 1 to 20 carbon atoms and branched or cyclic alkyl groups having 3 to 20 carbon atoms. Most preferably, R² is methyl.

R³ is preferably the same or different at each instance and is selected from H, D, F, CN, Si(R⁵)₃, N(R⁵)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where the alkyl groups mentioned, the aromatic ring systems mentioned and the heteroaromatic ring systems mentioned are each substituted by R⁵ radicals; and where one or more CH₂ groups in the alkyl groups mentioned may be replaced by —C═C—, —R⁵C═CR⁵—, Si(R⁵)₂, C═O, C═NR⁵, —NR⁵—, —O—, —S—, —C(═O)O— or —C(═O)NR⁵—. More preferably, R³ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals.

R⁴ is preferably the same or different at each instance and is selected from H, D, F, CN, Si(R⁵)₃, N(R⁵)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where the alkyl groups mentioned, the aromatic ring systems mentioned and the heteroaromatic ring systems mentioned are each substituted by R⁵ radicals; and where one or more CH₂ groups in the alkyl groups mentioned may be replaced by —C═C—, —R⁵C═CR⁵—, Si(R⁵)₂, C═O, C═NR⁵, —NR⁵—, —O—, —S—, —C(═O)O— or —C(═O)NR⁵—. More preferably, R⁴ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals.

R⁵ is preferably the same or different at each instance and is selected from H, D, F, CN, Si(R⁶)₃, N(R⁶)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where the alkyl groups mentioned, the aromatic ring systems mentioned and the heteroaromatic ring systems mentioned are each substituted by R⁶ radicals; and where one or more CH₂ groups in the alkyl groups mentioned may be replaced by —C═C—, —R⁶C═CR⁶—, Si(R⁶)₂, C═O, C═NR⁶, —NR⁶—, —O—, —S—, —C(═O)O— or —C(═O)NR⁶—. More preferably, R⁵ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁶ radicals.

R⁶ is preferably H.

Preferred embodiments of the formulae (G-1) to (G-6) are shown below:

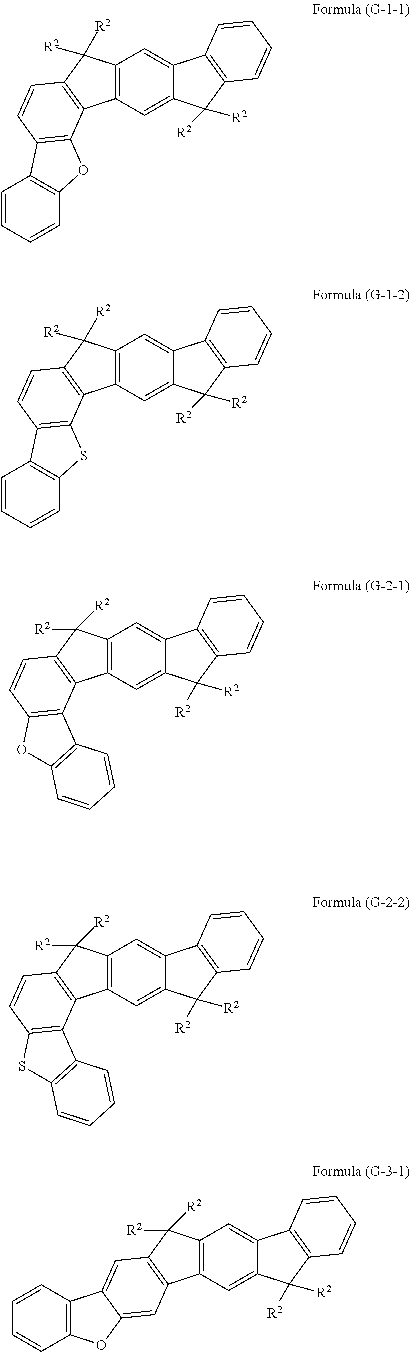

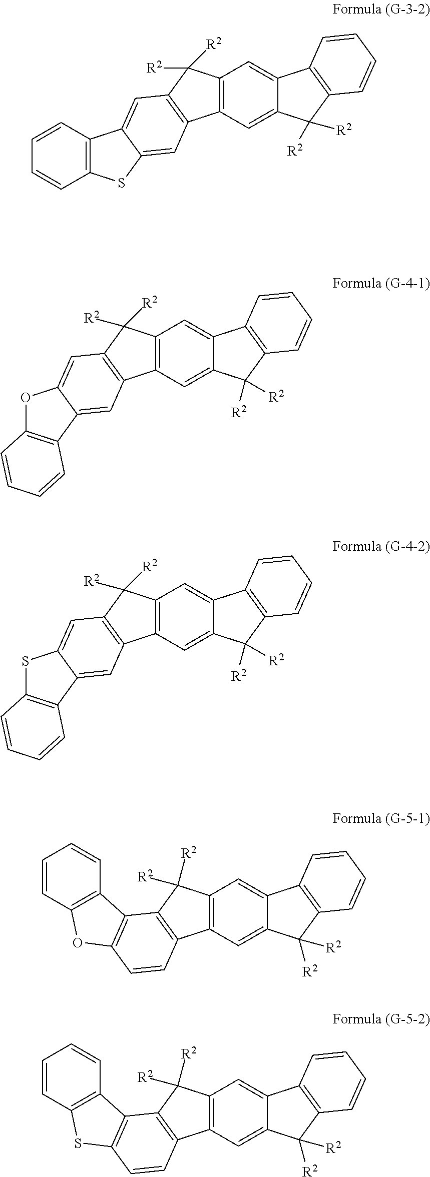

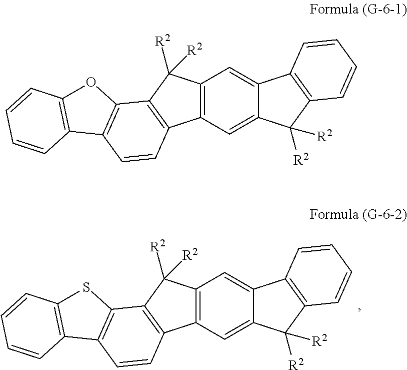

which are otherwise defined like formula (G) above. Among the formulae, formulae (G-1-1) and (G-1-2) are particularly preferred, very particularly formula (G-1-1).

Preferred embodiments of the formula (I) conform to the following formulae:

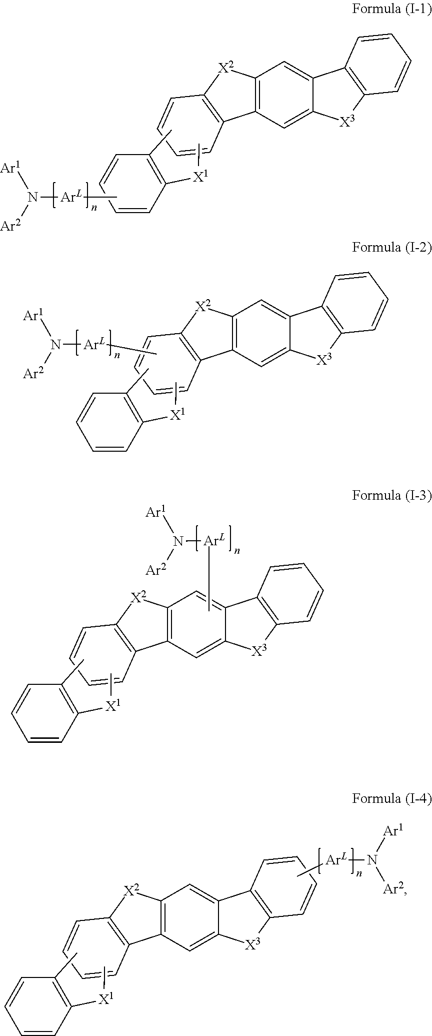

where the groups that occur are as defined above. Especially preferably, X² and X³ here are each C(R²)₂. Even more preferably, X¹ is O.

Preferred embodiments of the formulae (I-1) to (I-4) conform to the following formulae:

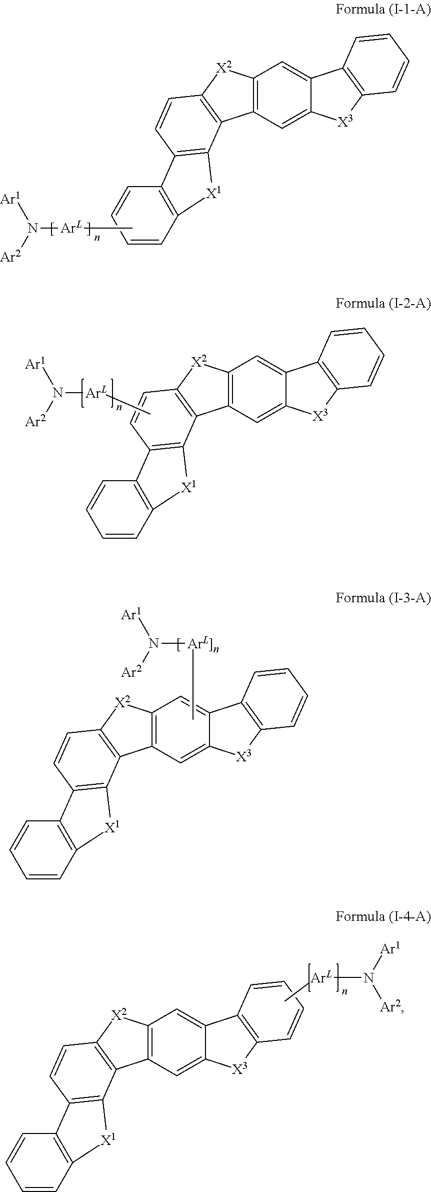

where the groups that occur are as defined above. Especially preferably, X² and X³ here are each C(R²)₂. Even more preferably, X¹ is O.

In a preferred embodiment, formula (I) conforms to one of formulae (I-1) and (I-3), especially formula (I-1). In a particularly preferred embodiment, formula (I) conforms to one of formulae (I-1-A) and (I-3-A), especially formula (I-1-A).

Preferred embodiments of the formula (I-1) conform to the following formulae:

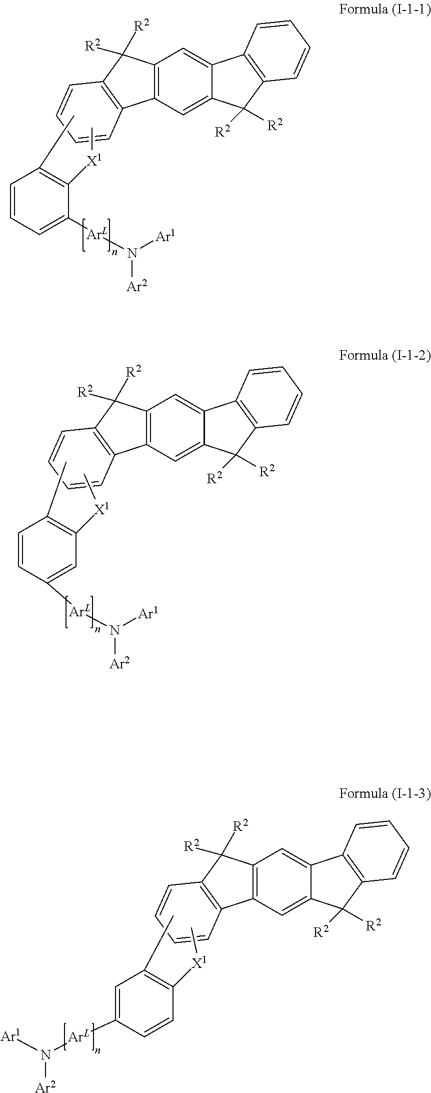

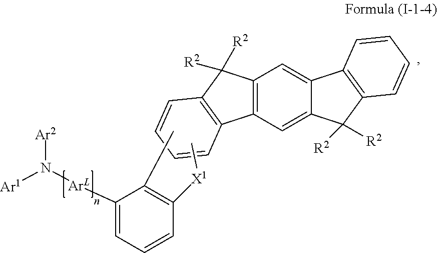

where the variables that occur are as defined above, and preferably correspond to their above-specified preferred embodiments. Among the formulae, particular preference is given to formula (I-1-1).

Particularly preferred embodiments of the formulae (I-1-1) to (I-1-4) are the following formulae:

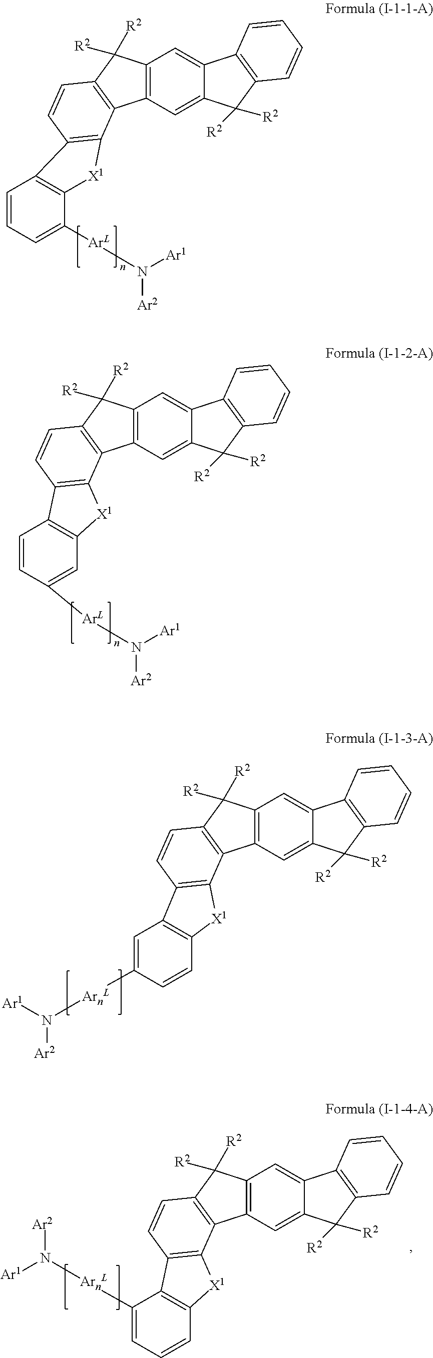

where the variables that occur are as defined above, and preferably correspond to their above-specified preferred embodiments. Among the formulae, particular preference is given to formula (I-1-1-A).

Preferred embodiments of the formula (I-2) conform to the following formulae:

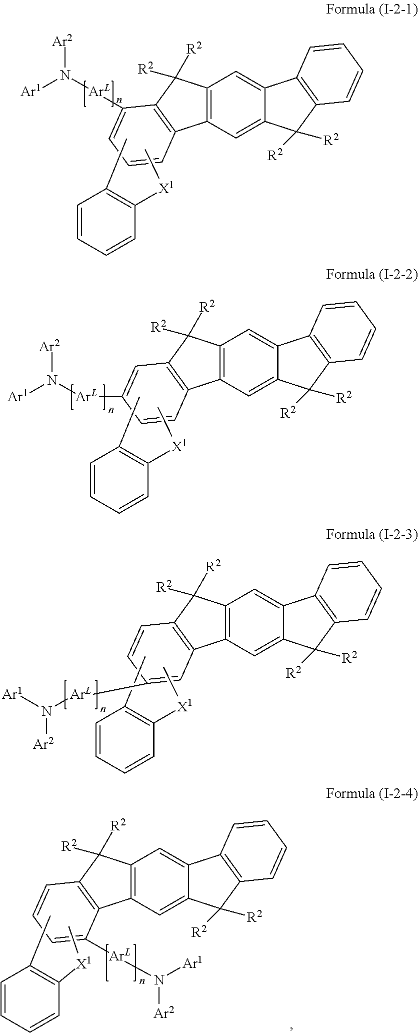

where the variables that occur are as defined above, and preferably correspond to their above-specified preferred embodiments. Among the formulae, particular preference is given to the formulae (I-2-1), (I-2-3) and (I-2-4).

Particularly preferred embodiments of the formulae (I-2-1) to (I-2-4) are the following formulae:

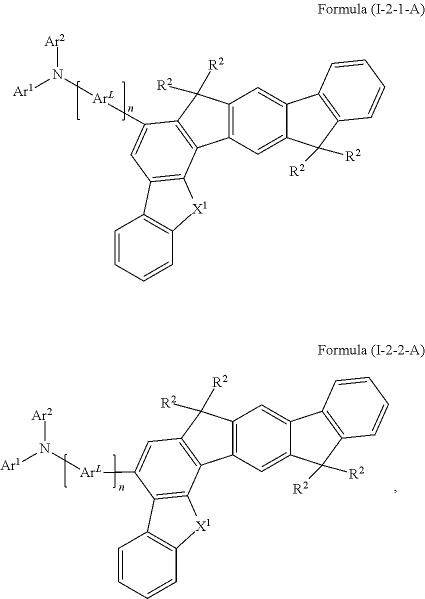

where the variables that occur are as defined above, and preferably correspond to their above-specified preferred embodiments. Among the formulae, particular preference is given to formula (I-2-1-A).

Preferred embodiments of the formula (I-3) conform to the following formulae:

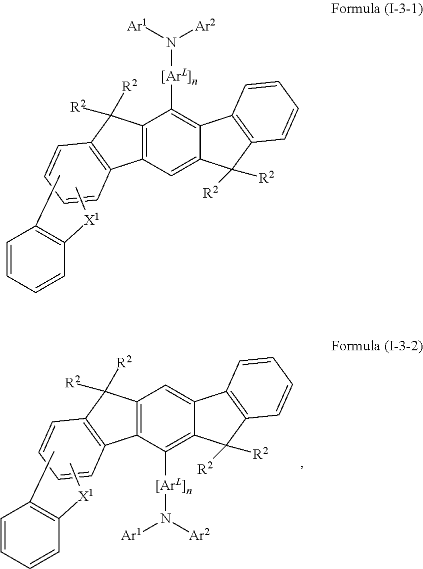

where the variables that occur are as defined above, and preferably correspond to their above-specified preferred embodiments.

Particularly preferred embodiments of the formulae (I-3-1) and (I-3-2) conform to the following formulae:

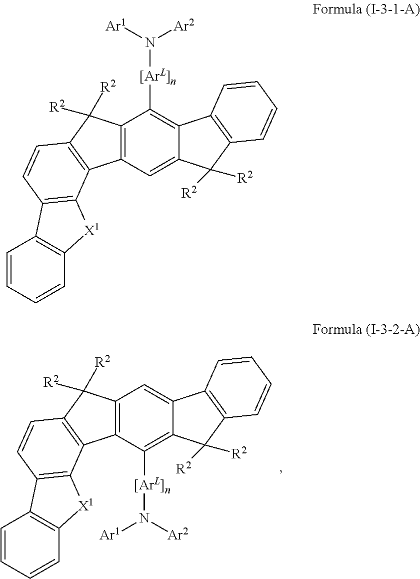

where the variables that occur are as defined above, and preferably correspond to their above-specified preferred embodiments.

Preferred embodiments of the formula (I-4) conform to the following formulae:

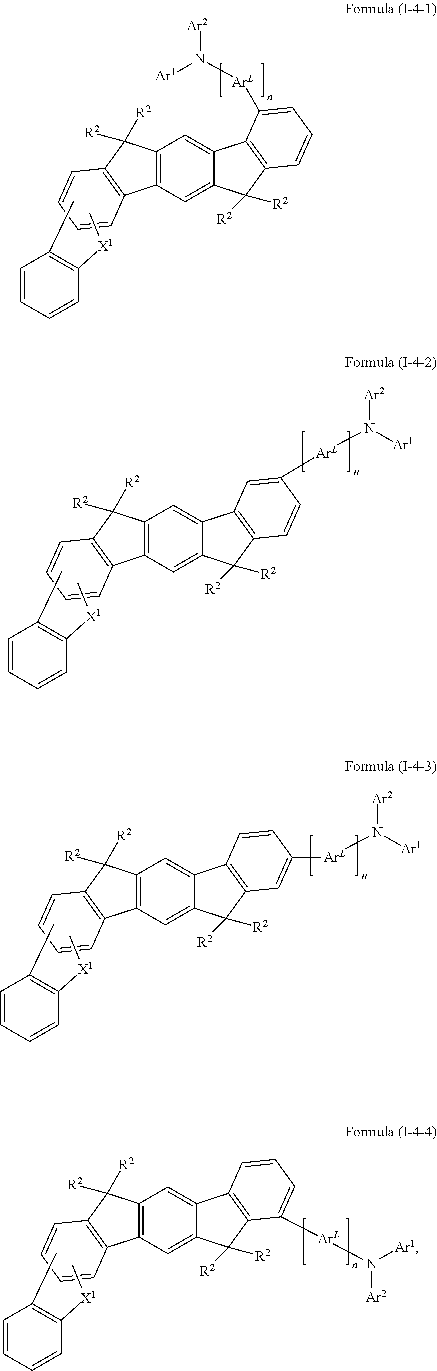

where the variables that occur are as defined above, and preferably correspond to their above-specified preferred embodiments. Among the formulae, particular preference is given to the formulae (I-4-1), (I-4-2) and (I-4-4). Very particular preference is given to the formula (I-4-1).

In an alternative preferred embodiment, formula (I) conforms to the formula (I-4-3).

Preferred embodiments of the formulae (I-4-1) to (I-4-4) conform to the following formulae:

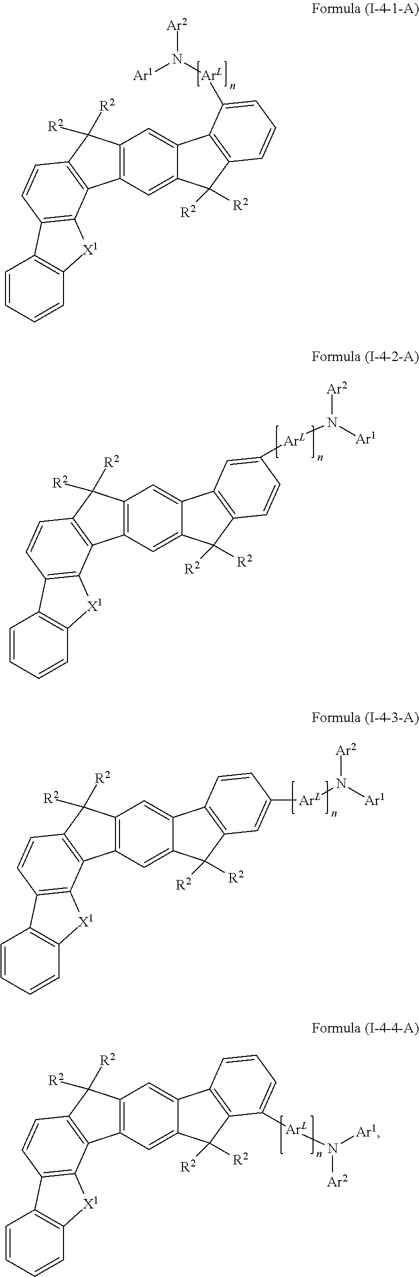

where the variables that occur are as defined above, and preferably correspond to their above-specified preferred embodiments. Among the formulae, formulae (I-4-1-A), (I-4-2-A) and (I-4-4-A) are particularly preferred; formula (I-4-1-A) is very particularly preferred.

In an alternative preferred embodiment, formula (I) conforms to the formula (I-4-3-A).

In a preferred embodiment, formula (I) conforms to one of formulae (I-1-1), (I-1-2), (I-1-3), (I-1-4), (I-3-1), (I-3-2), (I-4-1), (I-4-2) and (I-4-4). Among the formulae mentioned, preference is given in each case to the preferred embodiments of formulae (I-1-1-A), (I-1-2-A), (I-1-3-A), (I-1-4-A), (I-3-1-A), (I-3-2-A), (I-4-1-A), (I-4-2-A) and (I-4-4-A).

In an alternative preferred embodiment, formula (I) conforms to one of formulae (I-2-1) and (I-2-2), especially to formulae (I-2-1-A) and (I-2-2-A).

In a preferred embodiment, formula (I) conforms to one of formulae (I-1-1), (I-1-2), (I-1-3), (I-1-4), (I-3-1), (I-3-2). Among the formulae mentioned, preference is given in each case to the preferred embodiments of formulae (I-1-1-A), (I-1-2-A), (I-1-3-A), (I-1-4-A), (I-3-1-A), (I-3-2-A).

In a preferred embodiment, formula (I) conforms to one of formulae (I-4-1), (I-4-2) and (I-4-4), preferably to formula (I-4-1), especially to a formula (I-4-1-A), (I-4-2-A) and (I-4-4-A), preferably to formula (I-4-1-A), and where at least one of conditions a) and b) is applicable:
a) R² is the same or different at each instance and is selected from straight-chain alkyl groups having 1 to 20 carbon atoms and branched or cyclic alkyl groups having 3 to 20 carbon atoms; where two or more R² radicals may be joined to one another and may form a ring; where the alkyl groups mentioned may each be substituted by R⁵ radicals; and where one or more CH₂ groups in the alkyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
b) index n is 1.

In a preferred embodiment, both of conditions a) and b) are applicable.

In an alternative preferred embodiment, formula (I) conforms to formula (I-4-3), especially to formula (I-4-3-A), where at least one of conditions a) and b) exists:
a) R² is the same or different at each instance and is selected from straight-chain alkyl groups having 1 to 20 carbon atoms and branched or cyclic alkyl groups having 3 to 20 carbon atoms; where two or more R² radicals may be joined to one another and may form a ring; where the alkyl groups mentioned may each be substituted by R⁵ radicals; and where one or more CH₂ groups in the alkyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
b) index n is 1.

In a preferred embodiment, both of conditions a) and b) exist. Such compounds are especially well suited for use in a hole-transporting layer, which is also referred to as “common layer” in the terminology of the person skilled in the art, and which does not directly adjoin the emitting layer but adjoins a further hole-transporting layer on the anode side, which in turn adjoins the emitting layer on the anode side.

In a preferred embodiment of formula (I), the following definitions of the variables exist in combination:
G is a group of formula (G) which is bonded to the remainder of the formula (I) via one of the unoccupied positions on the benzene rings, where the other unoccupied positions on the benzene rings are each substituted by an R¹ radical;
X¹ is O or S;
X² is C(R²)₂;
X³ is C(R²)₂;
Ar^{L} is selected from phenyl, biphenyl, naphthyl and fluorenyl, which are each substituted by R³ radicals;
Ar¹, Ar² is the same or different and is an aromatic ring system which has 6 to 40 aromatic ring atoms and is substituted by R⁴ radicals, or a heteroaromatic ring system which has 5 to 25 aromatic ring atoms and is substituted by R⁴ radicals;
R¹ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals;
R² is the same or different at each instance and is selected from straight-chain alkyl groups having 1 to 20 carbon atoms and branched or cyclic alkyl groups having 3 to 20 carbon atoms;
R³ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals;
R⁴ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals;
R⁵ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁶ radicals, and where R⁶ radicals are H;
n is 0 or 1.

The present application also relates to a compound of the formula (I) as defined above, where, in a departure from the above definition, R² is as follows:

R² is the same or different and is selected from straight-chain alkyl groups having 1 to 20 carbon atoms and branched or cyclic alkyl groups having 3 to 20 carbon atoms; where two or more R² radicals may be joined to one another and may form a ring; where the alkyl groups mentioned are each substituted by R⁵ radicals; and where one or more CH₂ groups in the alkyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂; preferably, it is the same or different and is selected from straight-chain alkyl groups having 1 to 20 carbon atoms and branched or cyclic alkyl groups having 3 to 20 carbon atoms; more preferably, it is methyl.

All the other physical definitions given above in association with formula (I) are likewise considered to be preferred here. According to the application, the compound of the formula (I) with this definition of variable R² can be used generally in electronic devices, especially OLEDs, without the abovementioned restriction of obligatory use in a layer between anode and emitting layer.

The following compounds are preferred embodiments of compounds of the formula (I):

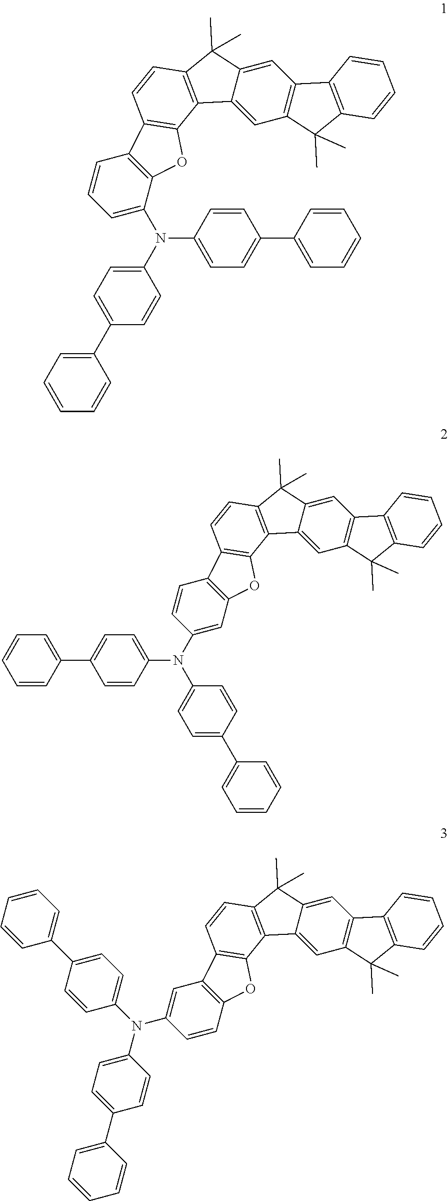 [SMILES: CC1(C)c2ccccc2-c2cc3c(cc21)-c1c(ccc2c1oc1c(N(c4ccc(-c5ccccc5)cc4)c4ccc(-c5ccccc5)cc4)cccc12)C3(C)C ; CC1(C)c2ccccc2-c2cc3c(cc21)-c1c(ccc2c1oc1cc(N(c4ccc(-c5ccccc5)cc4)c4ccc(-c5ccccc5)cc4)ccc12)C3(C)C ; CC1(C)c2ccccc2-c2cc3c(cc21)-c1c(ccc2c1oc1ccc(N(c4ccc(-c5ccccc5)cc4)c4ccc(-c5ccccc5)cc4)cc12)C3(C)C]

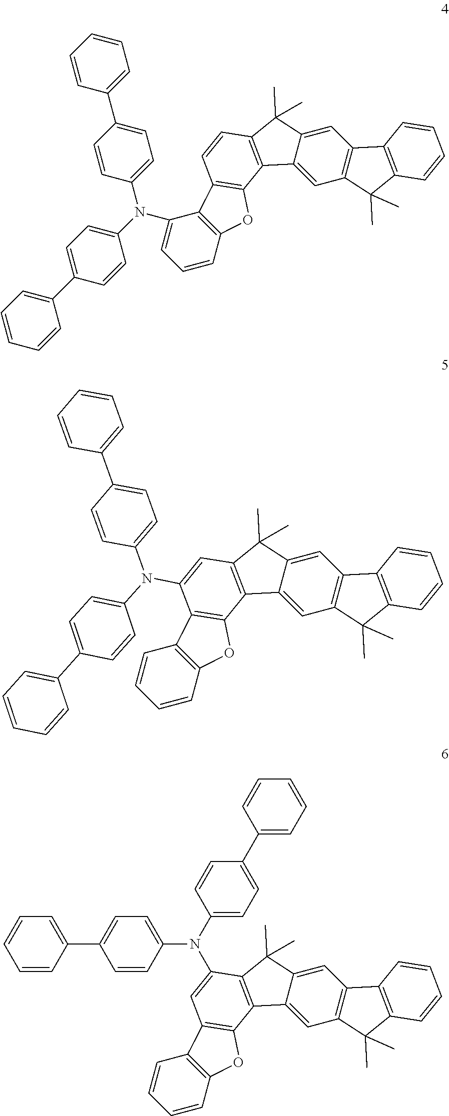 [SMILES: CC1(C)c2ccccc2-c2cc3c(cc21)-c1c(ccc2c1oc1cccc(N(c4ccc(-c5ccccc5)cc4)c4ccc(-c5ccccc5)cc4)c12)C3(C)C ; CC1(C)c2ccccc2-c2cc3c(cc21)-c1c(cc(N(c2ccc(-c4ccccc4)cc2)c2ccc(-c4ccccc4)cc2)c2c1oc1ccccc12)C3(C)C ; CC1(C)c2ccccc2-c2cc3c(cc21)-c1c(c(N(c2ccc(-c4ccccc4)cc2)c2ccc(-c4ccccc4)cc2)cc2c1oc1ccccc12)C3(C)C]

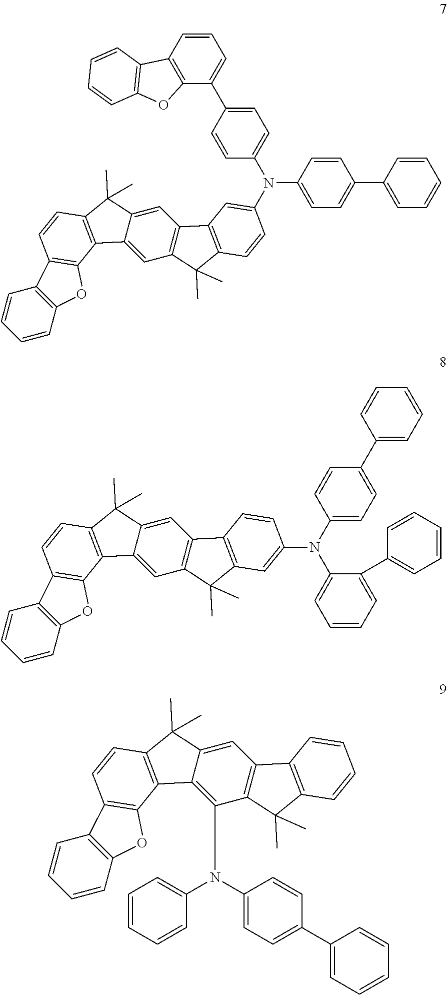 [SMILES: CC1(C)c2ccc(N(c3ccc(-c4ccccc4)cc3)c3ccc(-c4cccc5c4oc4ccccc45)cc3)cc2-c2cc3c(cc21)-c1c(ccc2c1oc1ccccc12)C3(C)C ; CC1(C)c2cc(N(c3ccc(-c4ccccc4)cc3)c3ccccc3-c3ccccc3)ccc2-c2cc3c(cc21)-c1c(ccc2c1oc1ccccc12)C3(C)C ; CC1(C)c2cc3c(c(N(c4ccccc4)c4ccc(-c5ccccc5)cc4)c2-c2c1ccc1c2oc2ccccc21)C(C)(C)c1ccccc1-3]

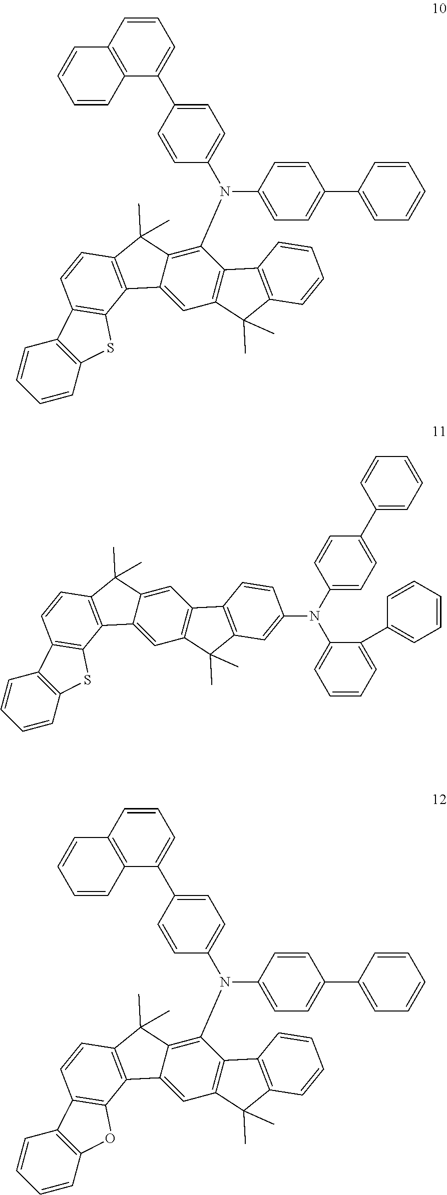 [SMILES: CC1(C)c2ccccc2-c2c1cc1c(c2N(c2ccc(-c3ccccc3)cc2)c2ccc(-c3cccc4ccccc34)cc2)C(C)(C)c2ccc3c(sc4ccccc43)c2-1 ; CC1(C)c2cc(N(c3ccc(-c4ccccc4)cc3)c3ccccc3-c3ccccc3)ccc2-c2cc3c(cc21)-c1c(ccc2c1sc1ccccc12)C3(C)C ; CC1(C)c2ccccc2-c2c1cc1c(c2N(c2ccc(-c3ccccc3)cc2)c2ccc(-c3cccc4ccccc34)cc2)C(C)(C)c2ccc3c(oc4ccccc43)c2-1]

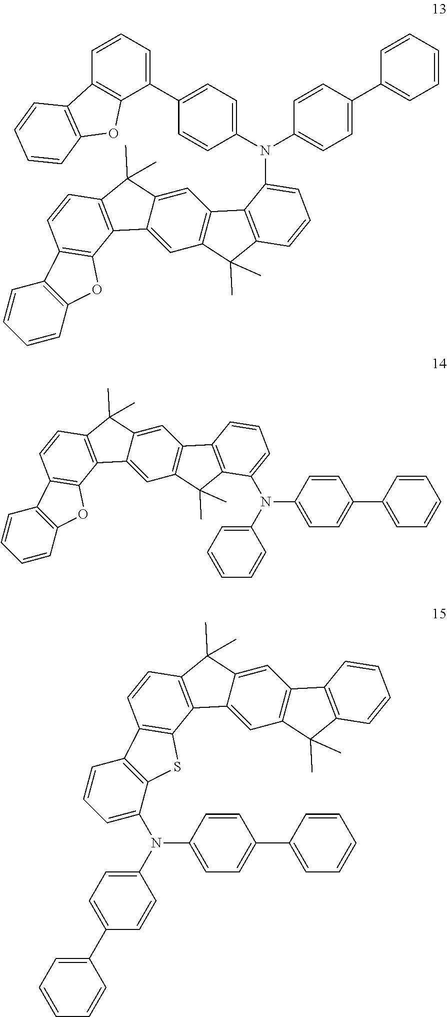 [SMILES: CC1(C)c2cc3c(cc2-c2c(N(c4ccc(-c5ccccc5)cc4)c4ccc(-c5cccc6c5oc5ccccc56)cc4)cccc21)C(C)(C)c1ccc2c(oc4ccccc42)c1-3 ; CC1(C)c2cc3c(cc2-c2c1ccc1c2oc2ccccc21)C(C)(C)c1c-3cccc1N(c1ccccc1)c1ccc(-c2ccccc2)cc1 ; CC1(C)c2ccccc2-c2cc3c(cc21)-c1c(ccc2c1sc1c(N(c4ccc(-c5ccccc5)cc4)c4ccc(-c5ccccc5)cc4)cccc12)C3(C)C]

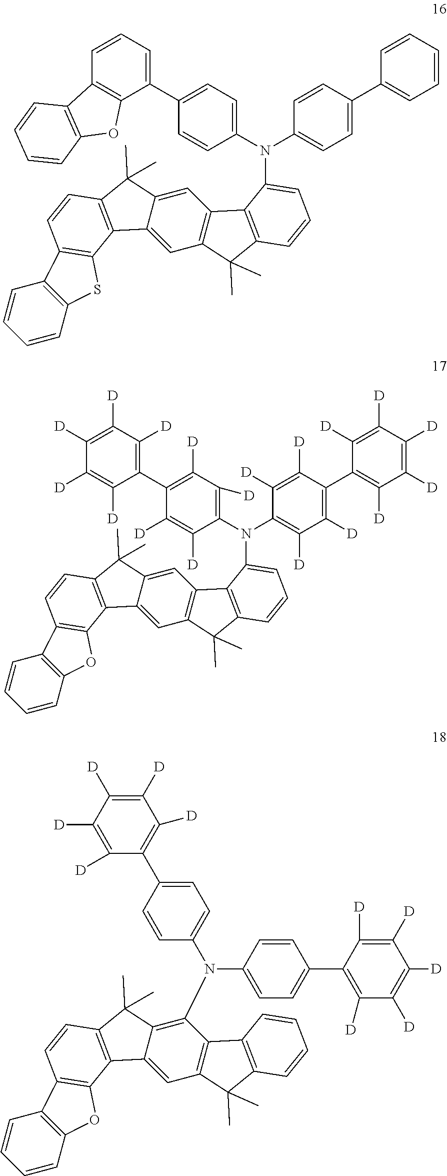 [SMILES: CC1(C)c2cc3c(cc2-c2c(N(c4ccc(-c5ccccc5)cc4)c4ccc(-c5cccc6c5oc5ccccc56)cc4)cccc21)C(C)(C)c1ccc2c(sc4ccccc42)c1-3 ; [2H]c1c([2H])c([2H])c(-c2c([2H])c([2H])c(N(c3cccc4c3-c3cc5c(cc3C4(C)C)-c3c(ccc4c3oc3ccccc34)C5(C)C)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c([2H])c(-c2ccc(N(c3ccc(-c4c([2H])c([2H])c([2H])c([2H])c4[2H])cc3)c3c4c(cc5c3C(C)(C)c3ccc6c(oc7ccccc76)c3-5)C(C)(C)c3ccccc3-4)cc2)c([2H])c1[2H]]

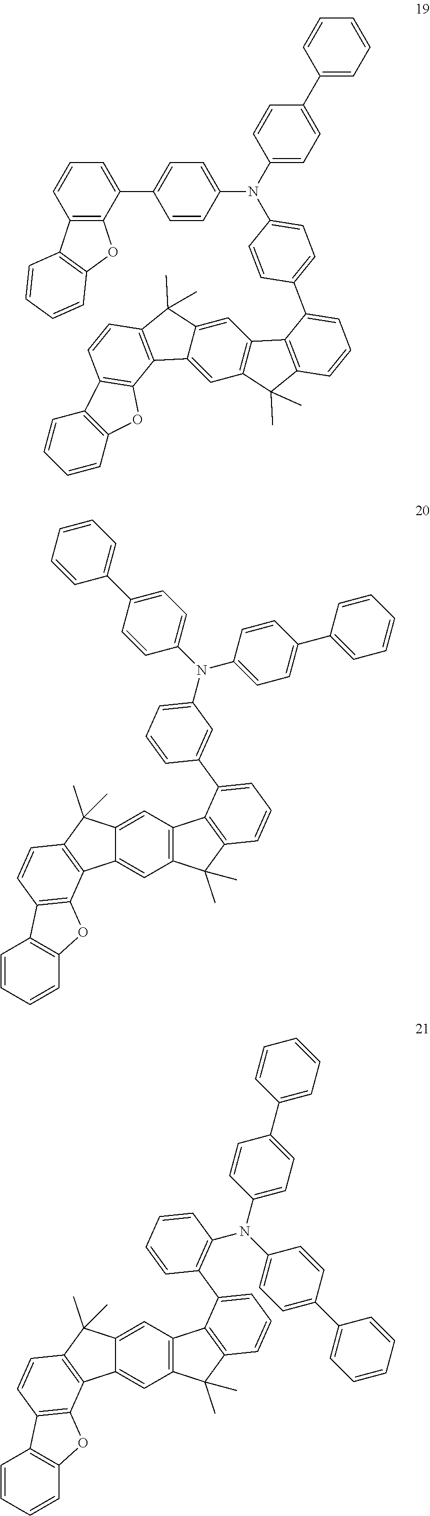 [SMILES: CC1(C)c2cc3c(cc2-c2c(-c4ccc(N(c5ccc(-c6ccccc6)cc5)c5ccc(-c6cccc7c6oc6ccccc67)cc5)cc4)cccc21)C(C)(C)c1ccc2c(oc4ccccc42)c1-3 ; CC1(C)c2cc3c(cc2-c2c(-c4cccc(N(c5ccc(-c6ccccc6)cc5)c5ccc(-c6ccccc6)cc5)c4)cccc21)C(C)(C)c1ccc2c(oc4ccccc42)c1-3 ; CC1(C)c2cc3c(cc2-c2c(-c4ccccc4N(c4ccc(-c5ccccc5)cc4)c4ccc(-c5ccccc5)cc4)cccc21)C(C)(C)c1ccc2c(oc4ccccc42)c1-3]

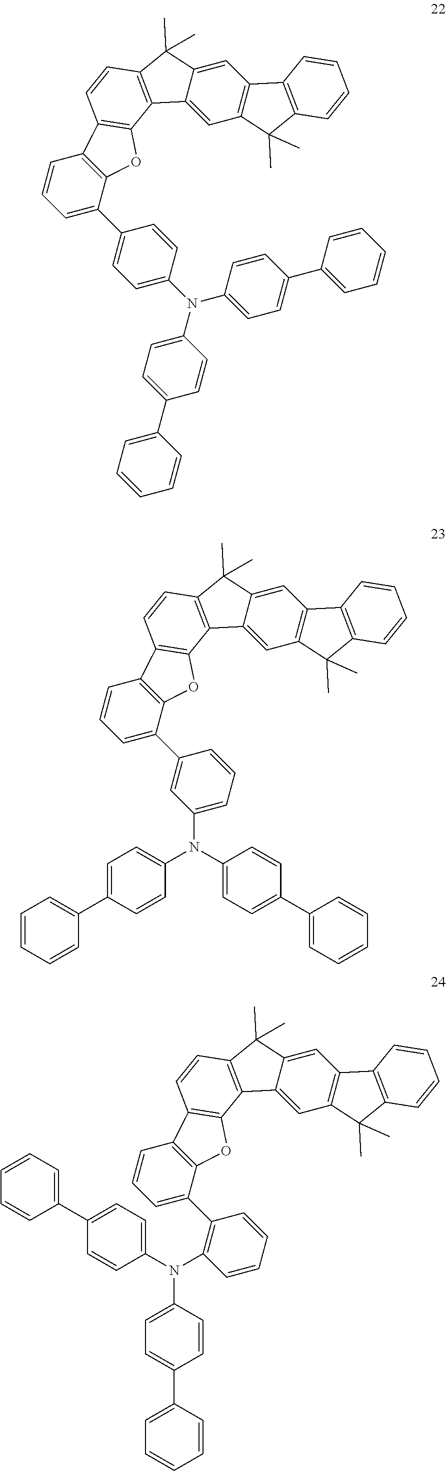 [SMILES: CC1(C)c2ccccc2-c2cc3c(cc21)-c1c(ccc2c1oc1c(-c4ccc(N(c5ccc(-c6ccccc6)cc5)c5ccc(-c6ccccc6)cc5)cc4)cccc12)C3(C)C ; CC1(C)c2ccccc2-c2cc3c(cc21)-c1c(ccc2c1oc1c(-c4cccc(N(c5ccc(-c6ccccc6)cc5)c5ccc(-c6ccccc6)cc5)c4)cccc12)C3(C)C ; CC1(C)c2ccccc2-c2cc3c(cc21)-c1c(ccc2c1oc1c(-c4ccccc4N(c4ccc(-c5ccccc5)cc4)c4ccc(-c5ccccc5)cc4)cccc12)C3(C)C]

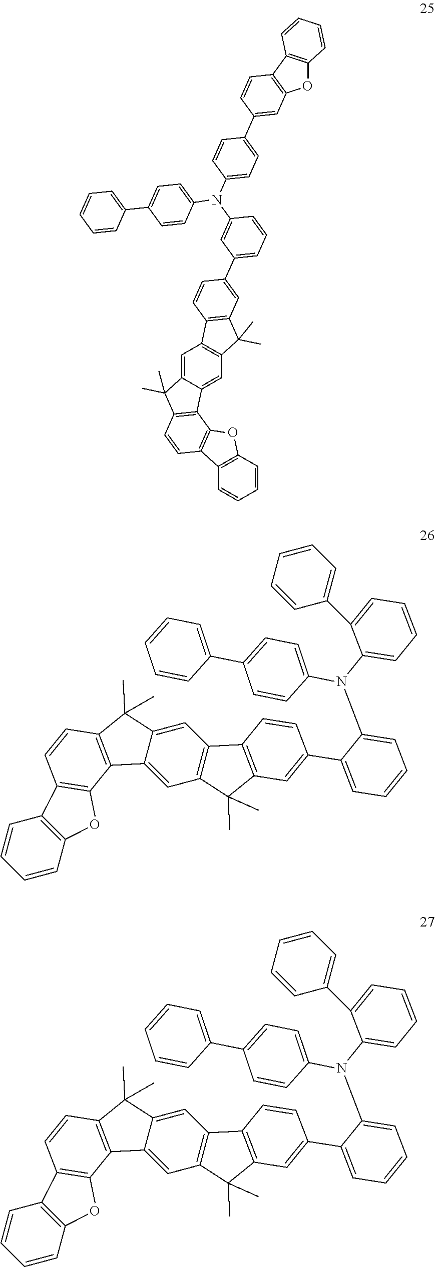 [SMILES: CC1(C)c2cc(-c3cccc(N(c4ccc(-c5ccccc5)cc4)c4ccc(-c5ccc6c(c5)oc5ccccc56)cc4)c3)ccc2-c2cc3c(cc21)-c1c(ccc2c1oc1ccccc12)C3(C)C ; CC1(C)c2cc(-c3ccccc3N(c3ccc(-c4ccccc4)cc3)c3ccccc3-c3ccccc3)ccc2-c2cc3c(cc21)-c1c(ccc2c1oc1ccccc12)C3(C)C ; CC1(C)c2cc(-c3ccccc3N(c3ccc(-c4ccccc4)cc3)c3ccccc3-c3ccccc3)ccc2-c2cc3c(cc21)-c1c(ccc2c1oc1ccccc12)C3(C)C]

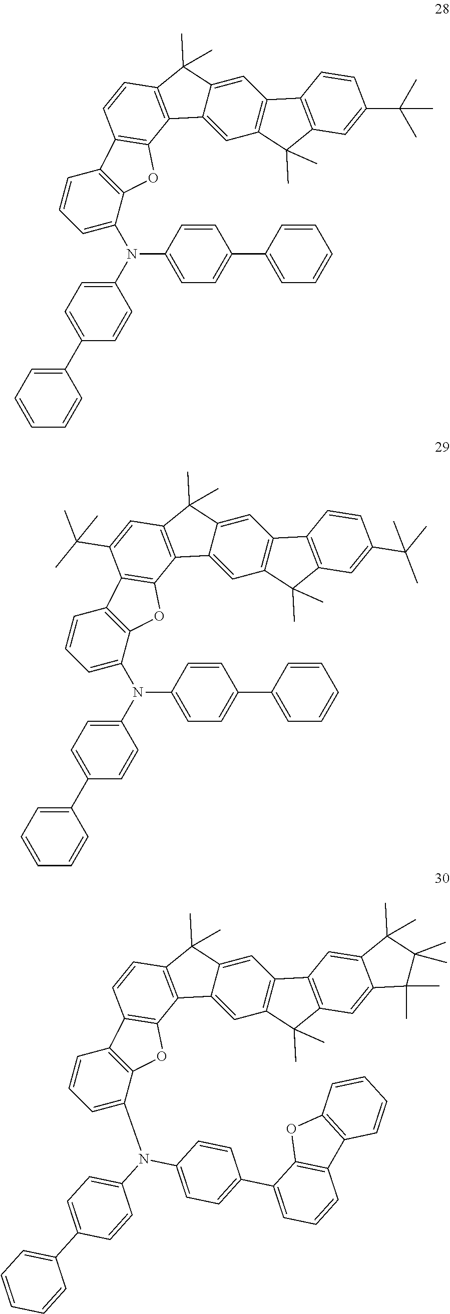 [SMILES: CC(C)(C)c1ccc2c(c1)C(C)(C)c1cc3c(cc1-2)C(C)(C)c1ccc2c(oc4c(N(c5ccc(-c6ccccc6)cc5)c5ccc(-c6ccccc6)cc5)cccc42)c1-3 ; CC(C)(C)c1ccc2c(c1)C(C)(C)c1cc3c(cc1-2)C(C)(C)c1cc(C(C)(C)C)c2c(oc4c(N(c5ccc(-c6ccccc6)cc5)c5ccc(-c6ccccc6)cc5)cccc42)c1-3 ; CC1(C)c2cc3c(cc2-c2cc4c(cc21)C(C)(C)C(C)(C)C4(C)C)C(C)(C)c1ccc2c(oc4c(N(c5ccc(-c6ccccc6)cc5)c5ccc(-c6cccc7c6oc6ccccc67)cc5)cccc42)c1-3]

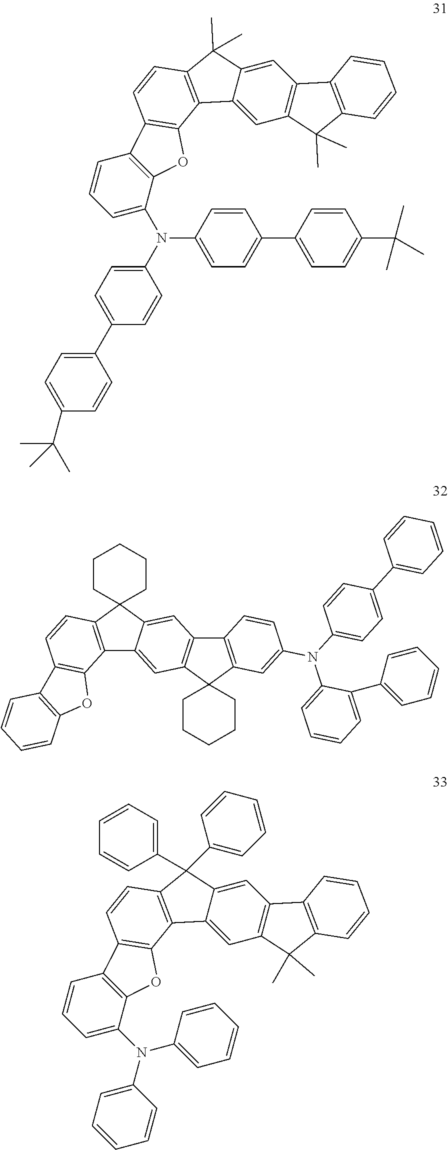 [SMILES: CC(C)(C)c1ccc(-c2ccc(N(c3ccc(-c4ccc(C(C)(C)C)cc4)cc3)c3cccc4c3oc3c5c(ccc34)C(C)(C)c3cc4c(cc3-5)C(C)(C)c3ccccc3-4)cc2)cc1 ; c1ccc(-c2ccc(N(c3ccc4c(c3)C3(CCCCC3)c3cc5c(cc3-4)C3(CCCCC3)c3ccc4c(oc6ccccc64)c3-5)c3ccccc3-c3ccccc3)cc2)cc1 ; CC1(C)c2ccccc2-c2cc3c(cc21)-c1c(ccc2c1oc1c(N(c4ccccc4)c4ccccc4)cccc12)C3(c1ccccc1)c1ccccc1]

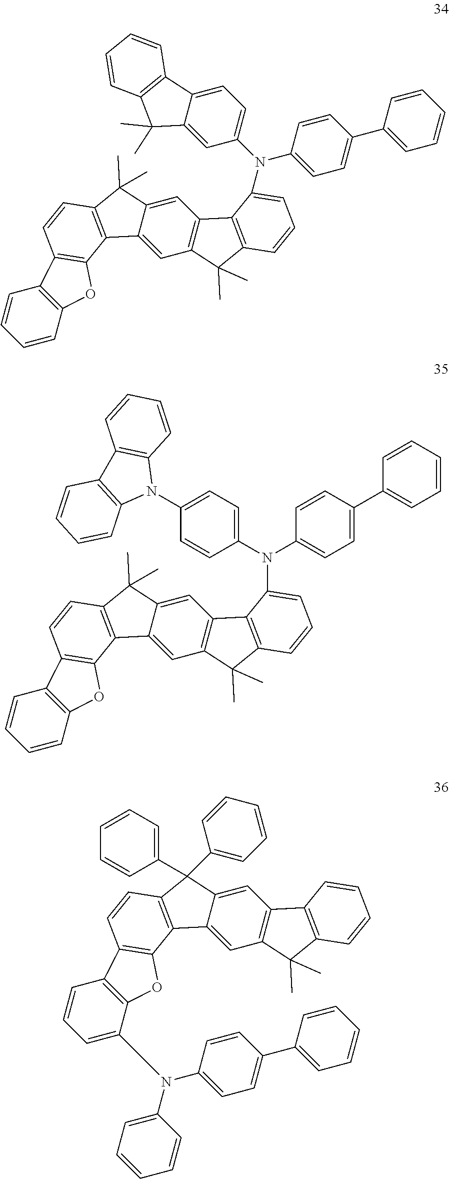 [SMILES: CC1(C)c2ccccc2-c2ccc(N(c3ccc(-c4ccccc4)cc3)c3cccc4c3-c3cc5c(cc3C4(C)C)-c3c(ccc4c3oc3ccccc34)C5(C)C)cc21 ; CC1(C)c2cc3c(cc2-c2c(N(c4ccc(-c5ccccc5)cc4)c4ccc(-n5c6ccccc6c6ccccc65)cc4)cccc21)C(C)(C)c1ccc2c(oc4ccccc42)c1-3 ; CC1(C)c2ccccc2-c2cc3c(cc21)-c1c(ccc2c1oc1c(N(c4ccccc4)c4ccc(-c5ccccc5)cc4)cccc12)C3(c1ccccc1)c1ccccc1]

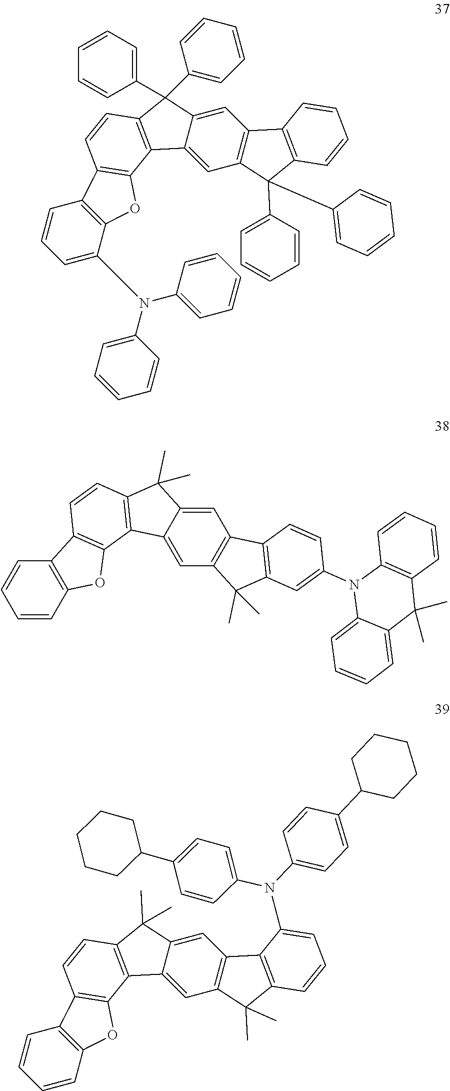 [SMILES: c1ccc(N(c2ccccc2)c2cccc3c2oc2c4c(ccc23)C(c2ccccc2)(c2ccccc2)c2cc3c(cc2-4)C(c2ccccc2)(c2ccccc2)c2ccccc2-3)cc1 ; CC1(C)c2cc(N3c4ccccc4C(C)(C)c4ccccc43)ccc2-c2cc3c(cc21)-c1c(ccc2c1oc1ccccc12)C3(C)C ; CC1(C)c2cc3c(cc2-c2c(N(c4ccc(C5CCCCC5)cc4)c4ccc(C5CCCCC5)cc4)cccc21)C(C)(C)c1ccc2c(oc4ccccc42)c1-3]

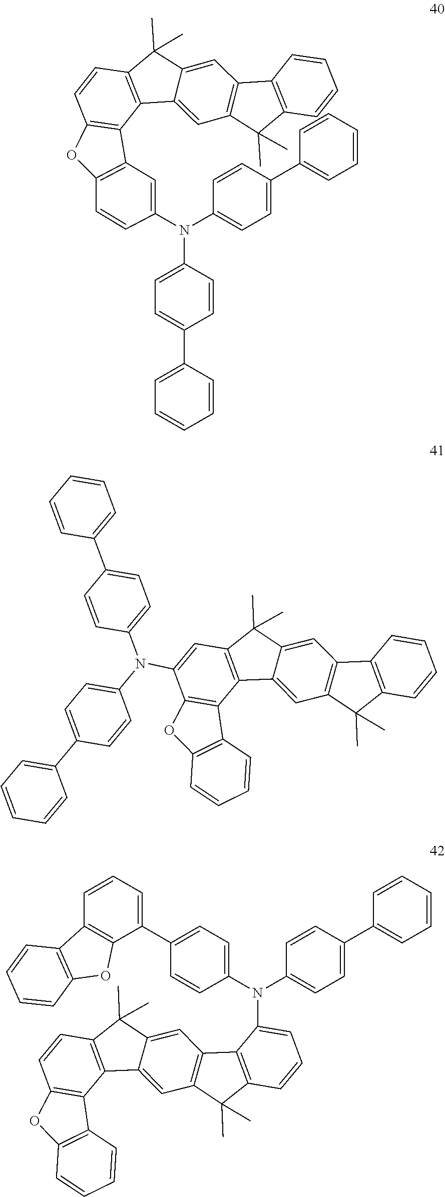 [SMILES: CC1(C)c2ccccc2-c2cc3c(cc21)-c1c(ccc2oc4ccc(N(c5ccc(-c6ccccc6)cc5)c5ccc(-c6ccccc6)cc5)cc4c12)C3(C)C ; CC1(C)c2ccccc2-c2cc3c(cc21)-c1c(cc(N(c2ccc(-c4ccccc4)cc2)c2ccc(-c4ccccc4)cc2)c2oc4ccccc4c12)C3(C)C ; CC1(C)c2cc3c(cc2-c2c(N(c4ccc(-c5ccccc5)cc4)c4ccc(-c5cccc6c5oc5ccccc56)cc4)cccc21)C(C)(C)c1ccc2oc4ccccc4c2c1-3]

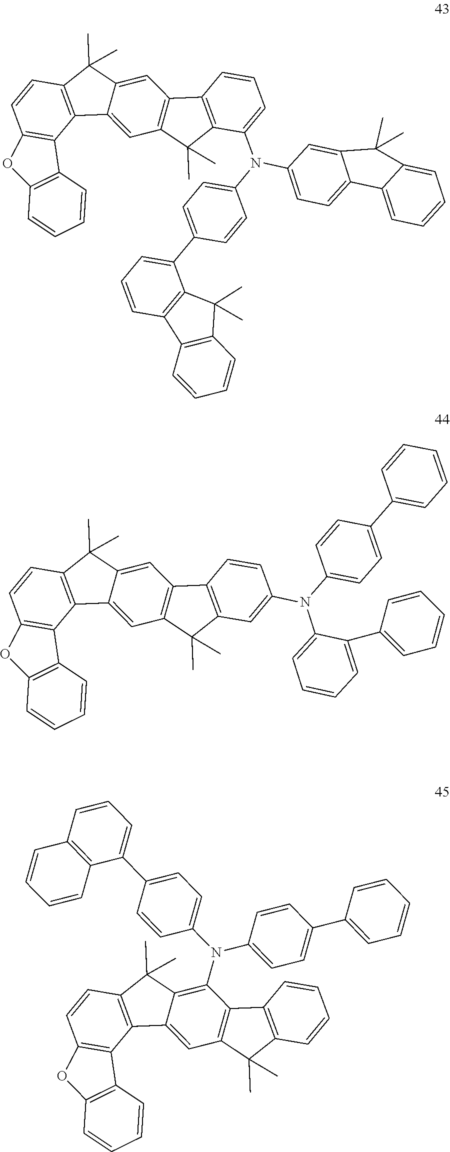 [SMILES: CC1(C)c2ccccc2-c2ccc(N(c3ccc(-c4cccc5c4C(C)(C)c4ccccc4-5)cc3)c3cccc4c3C(C)(C)c3cc5c(cc3-4)C(C)(C)c3ccc4oc6ccccc6c4c3-5)cc21 ; CC1(C)c2cc(N(c3ccc(-c4ccccc4)cc3)c3ccccc3-c3ccccc3)ccc2-c2cc3c(cc21)-c1c(ccc2oc4ccccc4c12)C3(C)C ; CC1(C)c2ccccc2-c2c1cc1c(c2N(c2ccc(-c3ccccc3)cc2)c2ccc(-c3cccc4ccccc34)cc2)C(C)(C)c2ccc3oc4ccccc4c3c2-1]

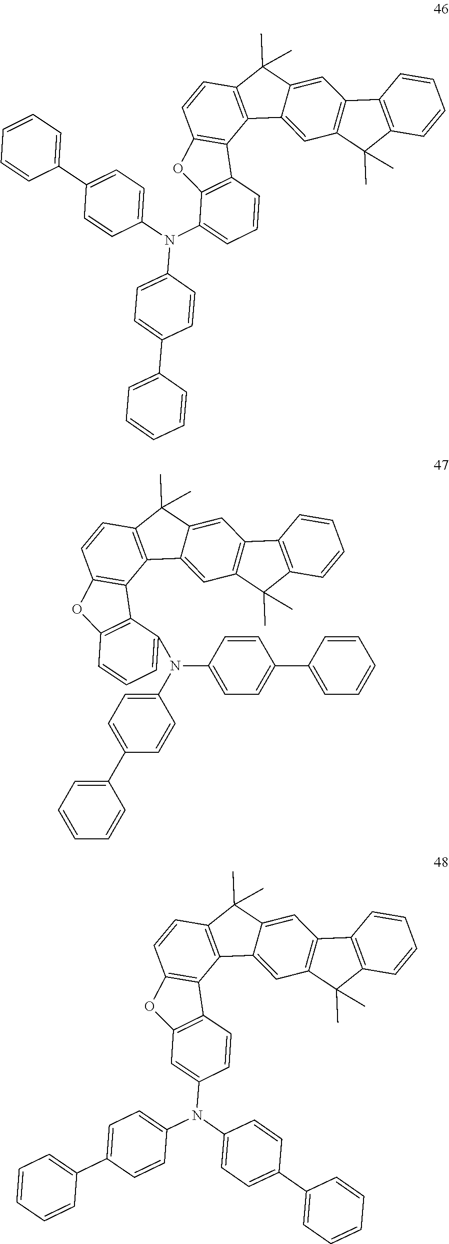

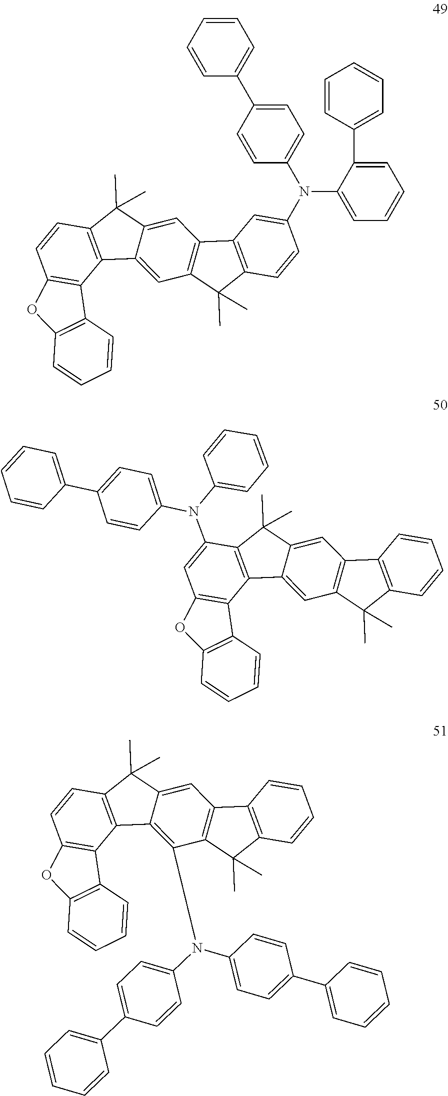 [SMILES: CC1(C)c2ccc(N(c3ccc(-c4ccccc4)cc3)c3ccccc3-c3ccccc3)cc2-c2cc3c(cc21)-c1c(ccc2oc4ccccc4c12)C3(C)C ; CC1(C)c2ccccc2-c2cc3c(cc21)-c1c(c(N(c2ccccc2)c2ccc(-c4ccccc4)cc2)cc2oc4ccccc4c12)C3(C)C ; CC1(C)c2cc3c(c(N(c4ccc(-c5ccccc5)cc4)c4ccc(-c5ccccc5)cc4)c2-c2c1ccc1oc4ccccc4c21)C(C)(C)c1ccccc1-3]

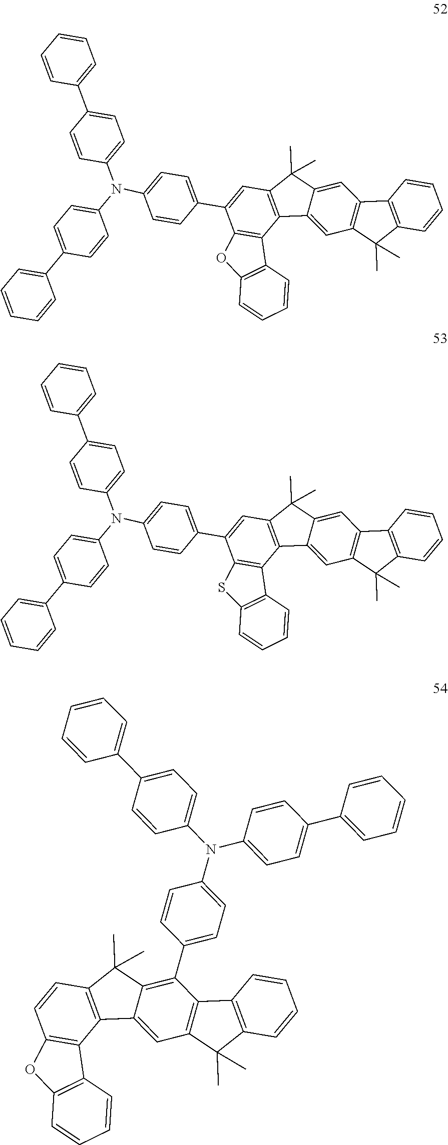 [SMILES: CC1(C)c2ccccc2-c2cc3c(cc21)-c1c(cc(-c2ccc(N(c4ccc(-c5ccccc5)cc4)c4ccc(-c5ccccc5)cc4)cc2)c2oc4ccccc4c12)C3(C)C ; CC1(C)c2ccccc2-c2cc3c(cc21)-c1c(cc(-c2ccc(N(c4ccc(-c5ccccc5)cc4)c4ccc(-c5ccccc5)cc4)cc2)c2sc4ccccc4c12)C3(C)C ; CC1(C)c2ccccc2-c2c1cc1c(c2-c2ccc(N(c3ccc(-c4ccccc4)cc3)c3ccc(-c4ccccc4)cc3)cc2)C(C)(C)c2ccc3oc4ccccc4c3c2-1]

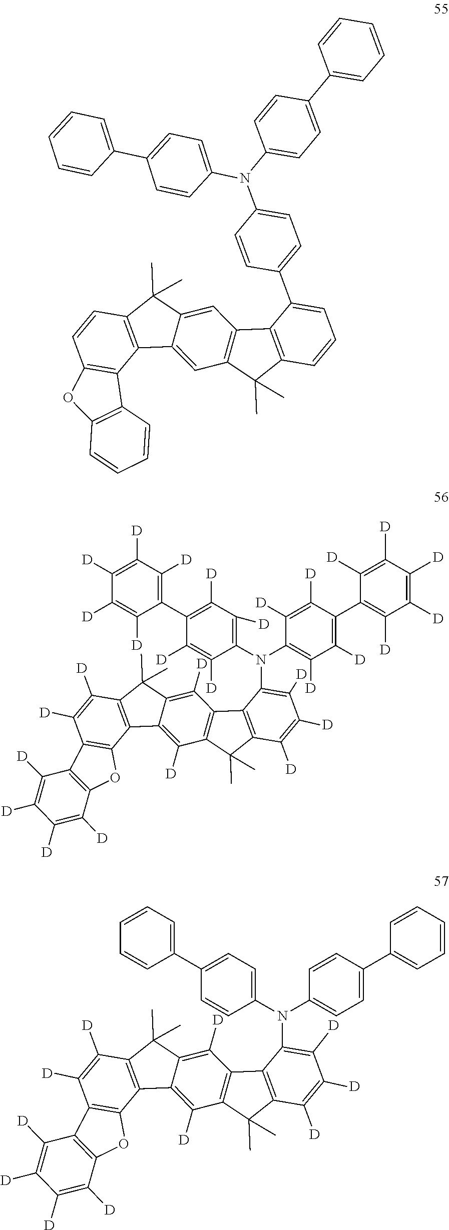

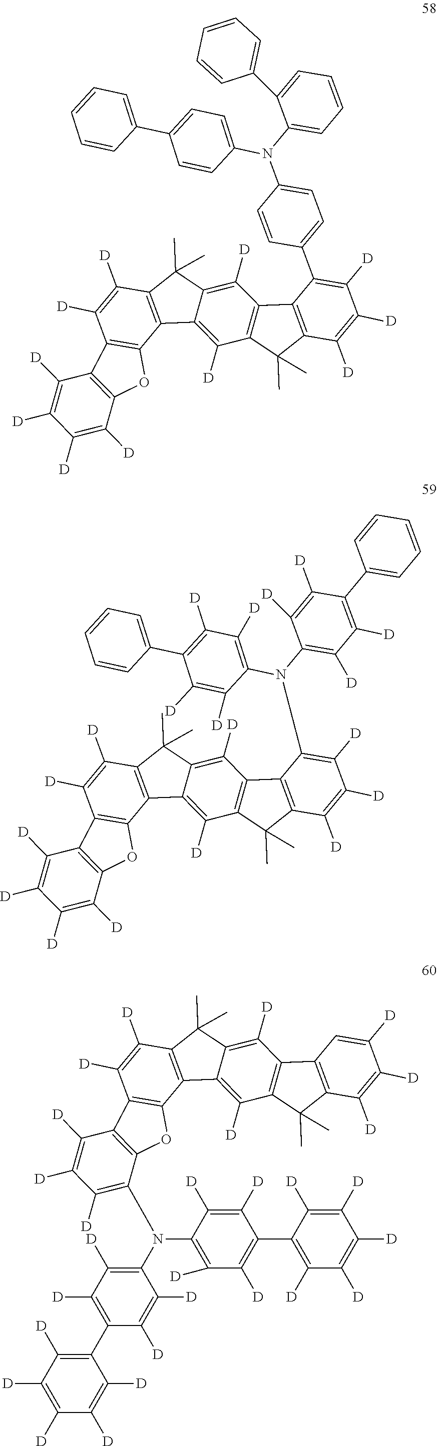

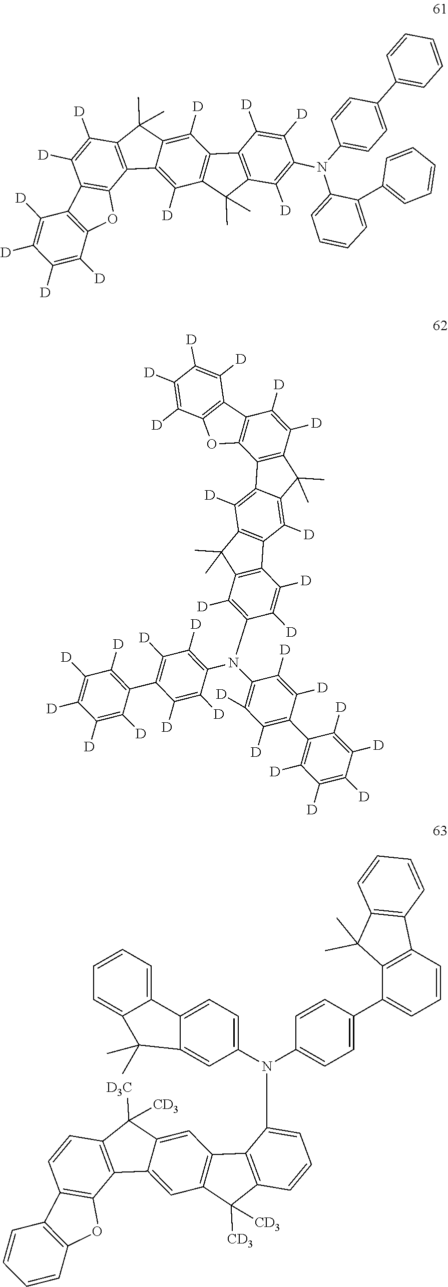

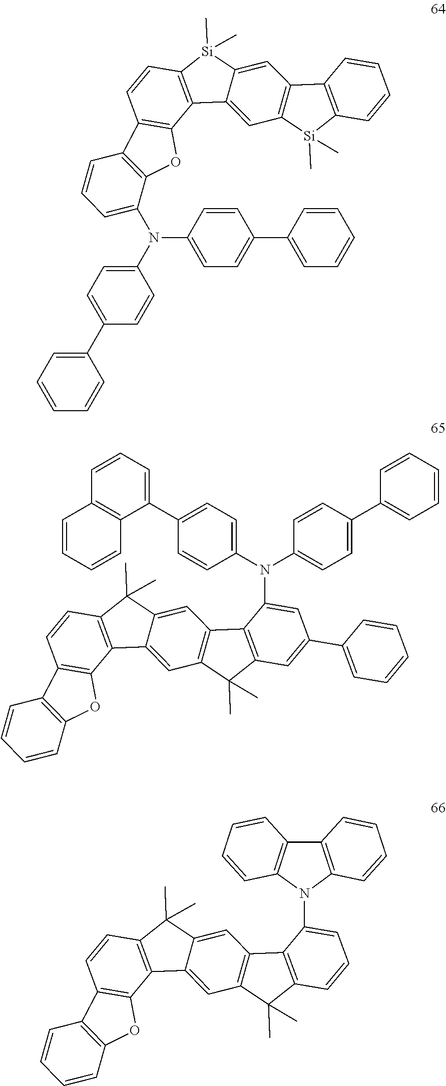 [SMILES: C[Si]1(C)c2ccccc2-c2cc3c(cc21)-c1c(ccc2c1oc1c(N(c4ccc(-c5ccccc5)cc4)c4ccc(-c5ccccc5)cc4)cccc12)[Si]3(C)C ; CC1(C)c2cc3c(cc2-c2c(N(c4ccc(-c5ccccc5)cc4)c4ccc(-c5cccc6ccccc56)cc4)cc(-c4ccccc4)cc21)C(C)(C)c1ccc2c(oc4ccccc42)c1-3 ; CC1(C)c2cc3c(cc2-c2c(-n4c5ccccc5c5ccccc54)cccc21)C(C)(C)c1ccc2c(oc4ccccc42)c1-3]

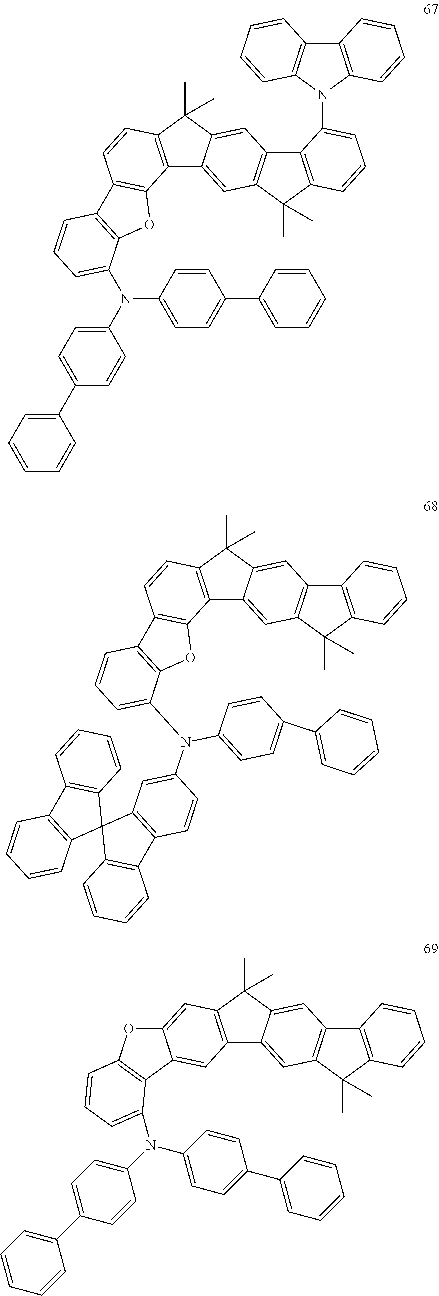 [SMILES: CC1(C)c2cc3c(cc2-c2c(-n4c5ccccc5c5ccccc54)cccc21)C(C)(C)c1ccc2c(oc4c(N(c5ccc(-c6ccccc6)cc5)c5ccc(-c6ccccc6)cc5)cccc42)c1-3 ; CC1(C)c2ccccc2-c2cc3c(cc21)-c1c(ccc2c1oc1c(N(c4ccc(-c5ccccc5)cc4)c4ccc5c(c4)C4(c6ccccc6-c6ccccc64)c4ccccc4-5)cccc12)C3(C)C ; CC1(C)c2ccccc2-c2cc3c(cc21)-c1cc2c(cc1C3(C)C)oc1cccc(N(c3ccc(-c4ccccc4)cc3)c3ccc(-c4ccccc4)cc3)c12]

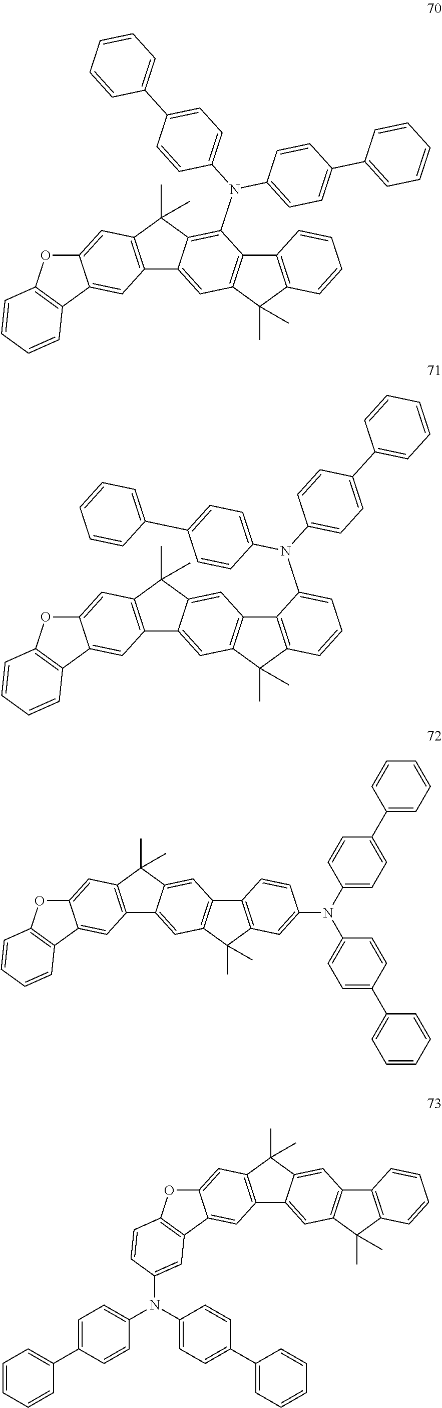 [SMILES: CC1(C)c2ccccc2-c2c1cc1c(c2N(c2ccc(-c3ccccc3)cc2)c2ccc(-c3ccccc3)cc2)C(C)(C)c2cc3oc4ccccc4c3cc2-1 ; CC1(C)c2cc3c(cc2-c2cc4c(cc21)oc1ccccc14)C(C)(C)c1cccc(N(c2ccc(-c4ccccc4)cc2)c2ccc(-c4ccccc4)cc2)c1-3 ; CC1(C)c2cc(N(c3ccc(-c4ccccc4)cc3)c3ccc(-c4ccccc4)cc3)ccc2-c2cc3c(cc21)-c1cc2c(cc1C3(C)C)oc1ccccc12 ; CC1(C)c2ccccc2-c2cc3c(cc21)-c1cc2c(cc1C3(C)C)oc1ccc(N(c3ccc(-c4ccccc4)cc3)c3ccc(-c4ccccc4)cc3)cc12]

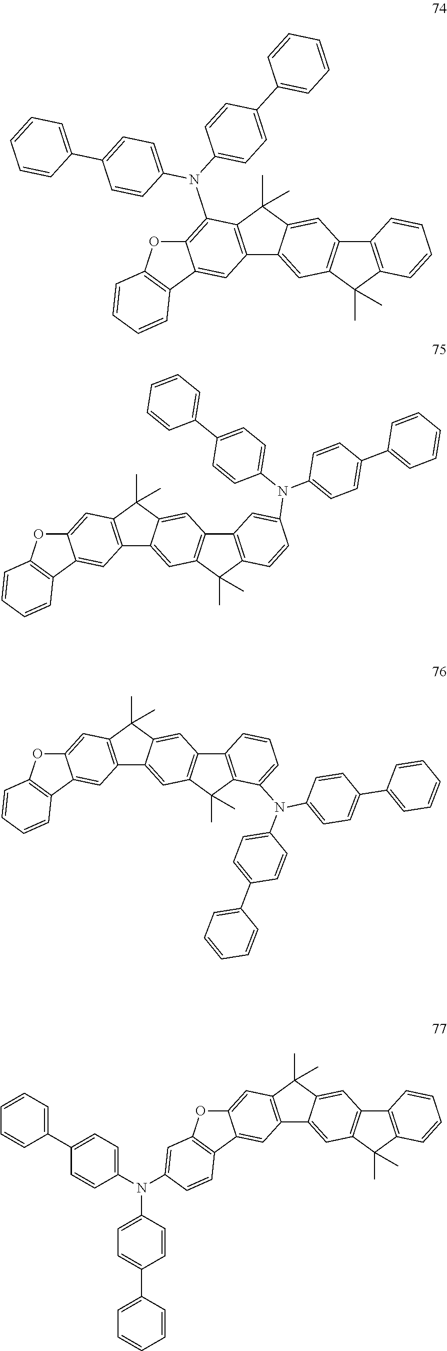 [SMILES: CC1(C)c2ccccc2-c2cc3c(cc21)-c1cc2c(oc4ccccc42)c(N(c2ccc(-c4ccccc4)cc2)c2ccc(-c4ccccc4)cc2)c1C3(C)C ; CC1(C)c2ccc(N(c3ccc(-c4ccccc4)cc3)c3ccc(-c4ccccc4)cc3)cc2-c2cc3c(cc21)-c1cc2c(cc1C3(C)C)oc1ccccc12 ; CC1(C)c2cc3c(cc2-c2cc4c(cc21)oc1ccccc14)C(C)(C)c1c-3cccc1N(c1ccc(-c2ccccc2)cc1)c1ccc(-c2ccccc2)cc1 ; CC1(C)c2ccccc2-c2cc3c(cc21)-c1cc2c(cc1C3(C)C)oc1cc(N(c3ccc(-c4ccccc4)cc3)c3ccc(-c4ccccc4)cc3)ccc12]

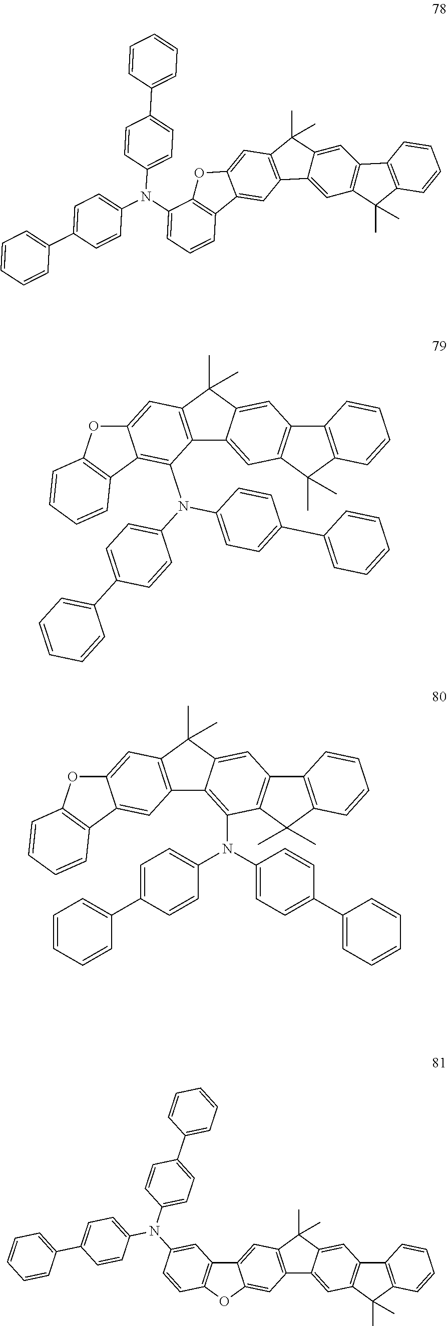 [SMILES: CC1(C)c2ccccc2-c2cc3c(cc21)-c1cc2c(cc1C3(C)C)oc1c(N(c3ccc(-c4ccccc4)cc3)c3ccc(-c4ccccc4)cc3)cccc12 ; CC1(C)c2ccccc2-c2cc3c(cc21)-c1c(cc2oc4ccccc4c2c1N(c1ccc(-c2ccccc2)cc1)c1ccc(-c2ccccc2)cc1)C3(C)C ; CC1(C)c2cc3oc4ccccc4c3cc2-c2c1cc1c(c2N(c2ccc(-c3ccccc3)cc2)c2ccc(-c3ccccc3)cc2)C(C)(C)c2ccccc2-1 ; CC1(C)c2ccccc2-c2cc3c(cc21)-c1cc2oc4ccc(N(c5ccc(-c6ccccc6)cc5)c5ccc(-c6ccccc6)cc5)cc4c2cc1C3(C)C]

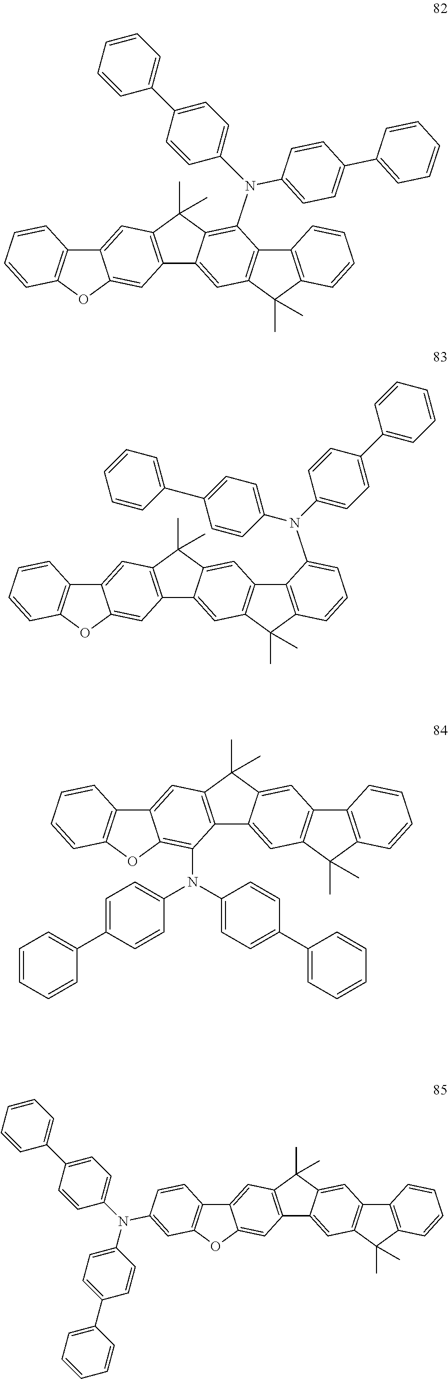 [SMILES: CC1(C)c2ccccc2-c2c1cc1c(c2N(c2ccc(-c3ccccc3)cc2)c2ccc(-c3ccccc3)cc2)C(C)(C)c2cc3c(cc2-1)oc1ccccc13 ; CC1(C)c2cc3c(cc2-c2cc4oc5ccccc5c4cc21)C(C)(C)c1cccc(N(c2ccc(-c4ccccc4)cc2)c2ccc(-c4ccccc4)cc2)c1-3 ; CC1(C)c2ccccc2-c2cc3c(cc21)-c1c(cc2c(oc4ccccc42)c1N(c1ccc(-c2ccccc2)cc1)c1ccc(-c2ccccc2)cc1)C3(C)C ; CC1(C)c2ccccc2-c2cc3c(cc21)-c1cc2oc4cc(N(c5ccc(-c6ccccc6)cc5)c5ccc(-c6ccccc6)cc5)ccc4c2cc1C3(C)C]

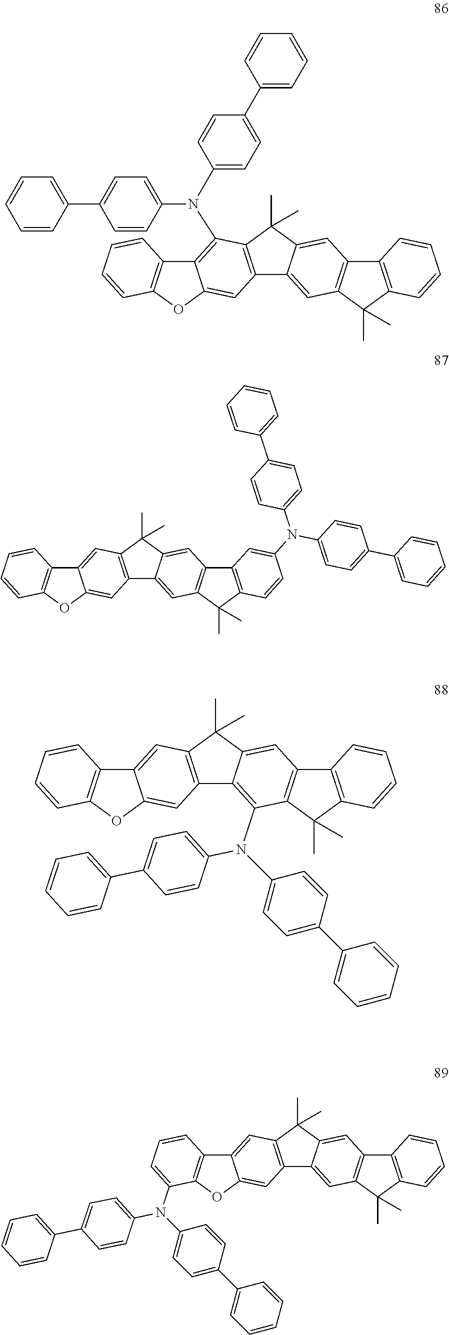 [SMILES: CC1(C)c2ccccc2-c2cc3c(cc21)-c1cc2oc4ccccc4c2c(N(c2ccc(-c4ccccc4)cc2)c2ccc(-c4ccccc4)cc2)c1C3(C)C ; CC1(C)c2ccc(N(c3ccc(-c4ccccc4)cc3)c3ccc(-c4ccccc4)cc3)cc2-c2cc3c(cc21)-c1cc2oc4ccccc4c2cc1C3(C)C ; CC1(C)c2cc3c(cc2-c2c1cc1c(c2N(c2ccc(-c4ccccc4)cc2)c2ccc(-c4ccccc4)cc2)C(C)(C)c2ccccc2-1)oc1ccccc13 ; CC1(C)c2ccccc2-c2cc3c(cc21)-c1cc2oc4c(N(c5ccc(-c6ccccc6)cc5)c5ccc(-c6ccccc6)cc5)cccc4c2cc1C3(C)C]

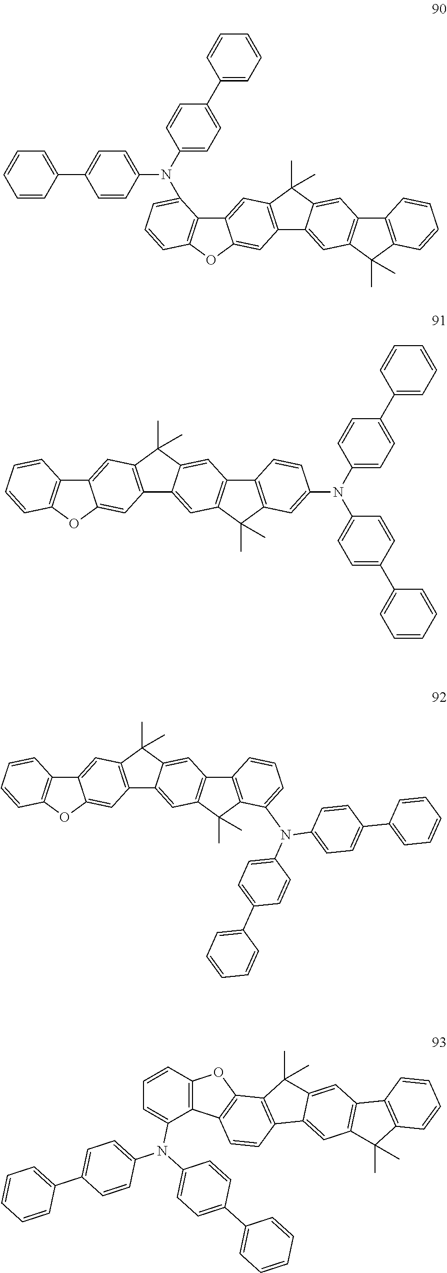 [SMILES: CC1(C)c2ccccc2-c2cc3c(cc21)-c1cc2oc4cccc(N(c5ccc(-c6ccccc6)cc5)c5ccc(-c6ccccc6)cc5)c4c2cc1C3(C)C ; CC1(C)c2cc(N(c3ccc(-c4ccccc4)cc3)c3ccc(-c4ccccc4)cc3)ccc2-c2cc3c(cc21)-c1cc2oc4ccccc4c2cc1C3(C)C ; CC1(C)c2cc3c(cc2-c2cc4oc5ccccc5c4cc21)C(C)(C)c1c-3cccc1N(c1ccc(-c2ccccc2)cc1)c1ccc(-c2ccccc2)cc1 ; CC1(C)c2ccccc2-c2cc3c(cc21)-c1ccc2c(oc4cccc(N(c5ccc(-c6ccccc6)cc5)c5ccc(-c6ccccc6)cc5)c42)c1C3(C)C]

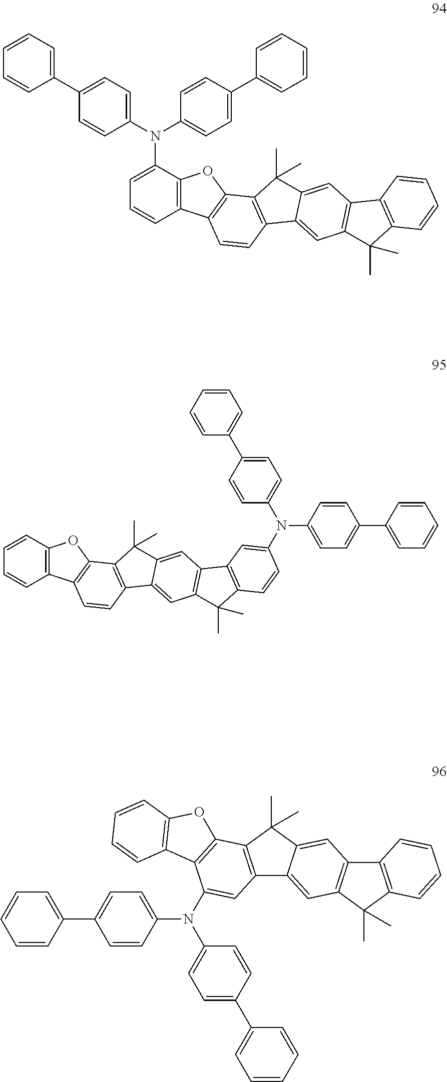 [SMILES: CC1(C)c2ccccc2-c2cc3c(cc21)-c1ccc2c(oc4c(N(c5ccc(-c6ccccc6)cc5)c5ccc(-c6ccccc6)cc5)cccc42)c1C3(C)C ; CC1(C)c2ccc(N(c3ccc(-c4ccccc4)cc3)c3ccc(-c4ccccc4)cc3)cc2-c2cc3c(cc21)-c1ccc2c(oc4ccccc42)c1C3(C)C ; CC1(C)c2ccccc2-c2cc3c(cc21)-c1cc(N(c2ccc(-c4ccccc4)cc2)c2ccc(-c4ccccc4)cc2)c2c(oc4ccccc42)c1C3(C)C]

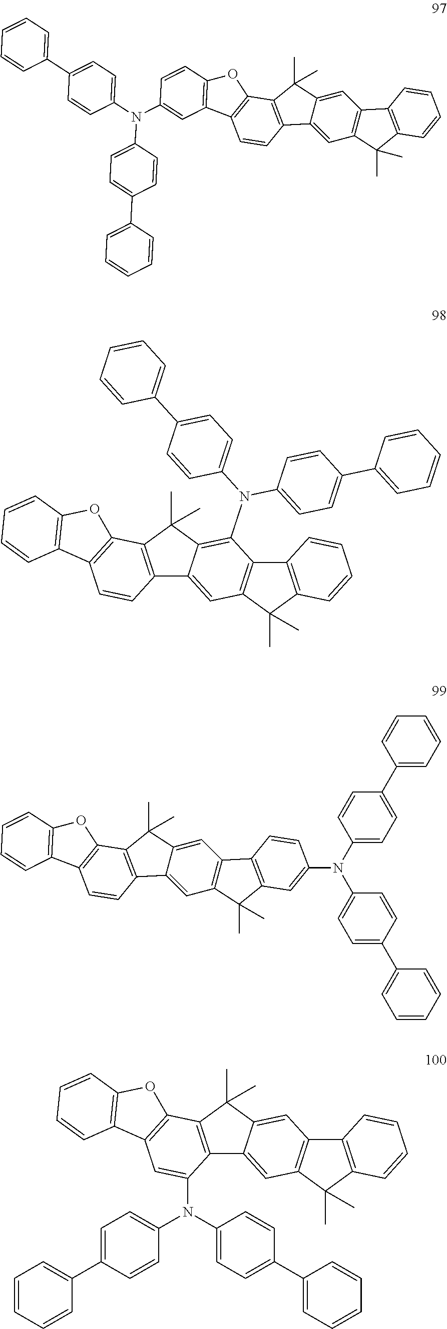 [SMILES: CC1(C)c2ccccc2-c2cc3c(cc21)-c1ccc2c(oc4ccc(N(c5ccc(-c6ccccc6)cc5)c5ccc(-c6ccccc6)cc5)cc42)c1C3(C)C ; CC1(C)c2ccccc2-c2c1cc1c(c2N(c2ccc(-c3ccccc3)cc2)c2ccc(-c3ccccc3)cc2)C(C)(C)c2c-1ccc1c2oc2ccccc21 ; CC1(C)c2cc(N(c3ccc(-c4ccccc4)cc3)c3ccc(-c4ccccc4)cc3)ccc2-c2cc3c(cc21)-c1ccc2c(oc4ccccc42)c1C3(C)C ; CC1(C)c2ccccc2-c2cc3c(cc21)-c1c(N(c2ccc(-c4ccccc4)cc2)c2ccc(-c4ccccc4)cc2)cc2c(oc4ccccc42)c1C3(C)C]

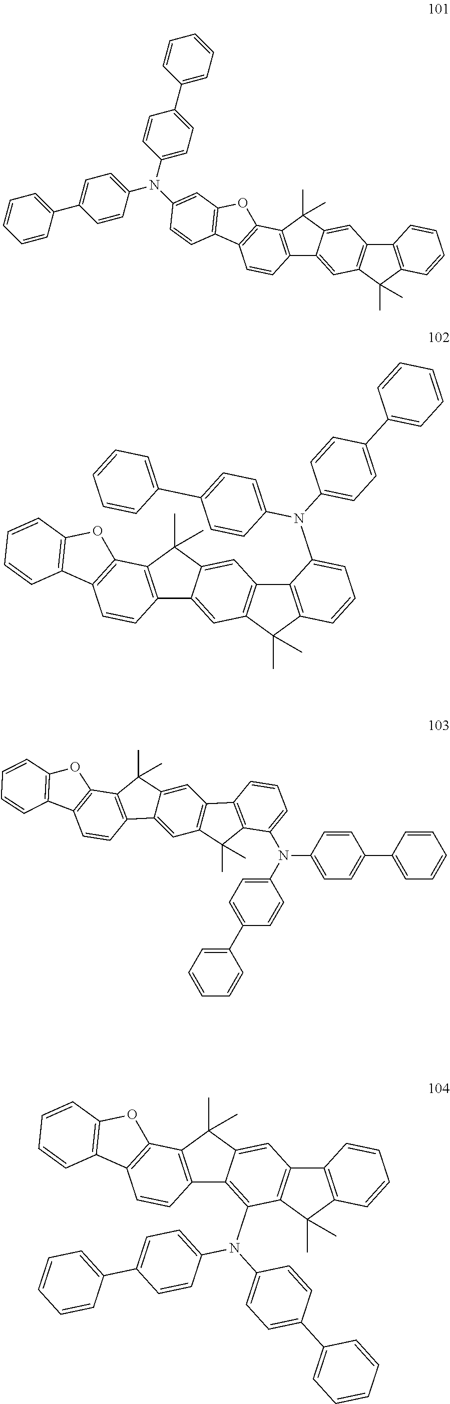 [SMILES: CC1(C)c2ccccc2-c2cc3c(cc21)-c1ccc2c(oc4cc(N(c5ccc(-c6ccccc6)cc5)c5ccc(-c6ccccc6)cc5)ccc42)c1C3(C)C ; CC1(C)c2cc3c(cc2-c2c(N(c4ccc(-c5ccccc5)cc4)c4ccc(-c5ccccc5)cc4)cccc21)C(C)(C)c1c-3ccc2c1oc1ccccc12 ; CC1(C)c2cc3c(cc2-c2cccc(N(c4ccc(-c5ccccc5)cc4)c4ccc(-c5ccccc5)cc4)c21)C(C)(C)c1c-3ccc2c1oc1ccccc12 ; CC1(C)c2ccccc2-c2cc3c(c(N(c4ccc(-c5ccccc5)cc4)c4ccc(-c5ccccc5)cc4)c21)-c1ccc2c(oc4ccccc42)c1C3(C)C]

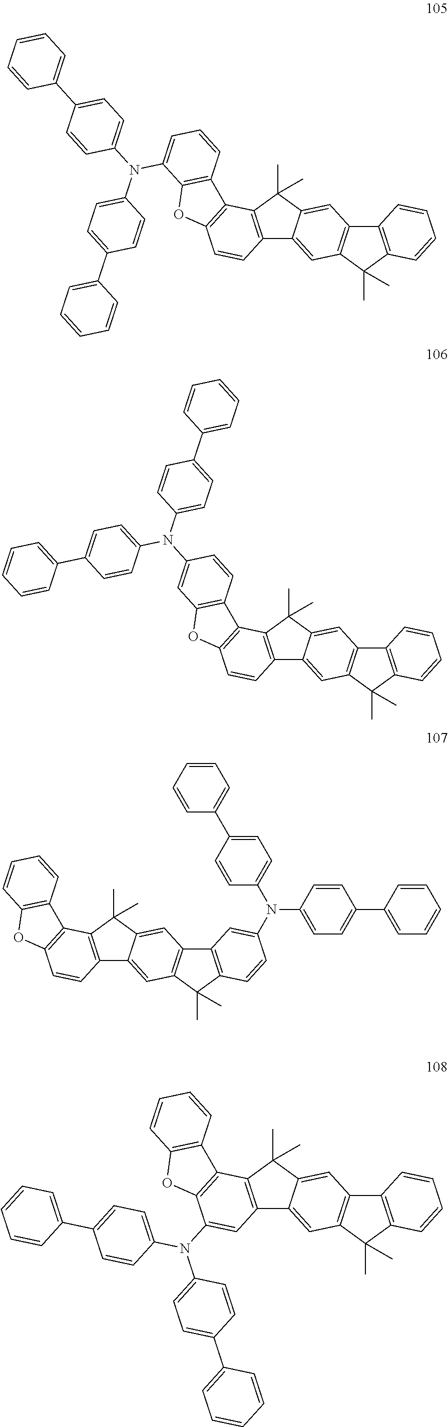 [SMILES: CC1(C)c2ccccc2-c2cc3c(cc21)-c1ccc2oc4c(N(c5ccc(-c6ccccc6)cc5)c5ccc(-c6ccccc6)cc5)cccc4c2c1C3(C)C ; CC1(C)c2ccccc2-c2cc3c(cc21)-c1ccc2oc4cc(N(c5ccc(-c6ccccc6)cc5)c5ccc(-c6ccccc6)cc5)ccc4c2c1C3(C)C ; CC1(C)c2ccc(N(c3ccc(-c4ccccc4)cc3)c3ccc(-c4ccccc4)cc3)cc2-c2cc3c(cc21)-c1ccc2oc4ccccc4c2c1C3(C)C ; CC1(C)c2ccccc2-c2cc3c(cc21)-c1cc(N(c2ccc(-c4ccccc4)cc2)c2ccc(-c4ccccc4)cc2)c2oc4ccccc4c2c1C3(C)C]

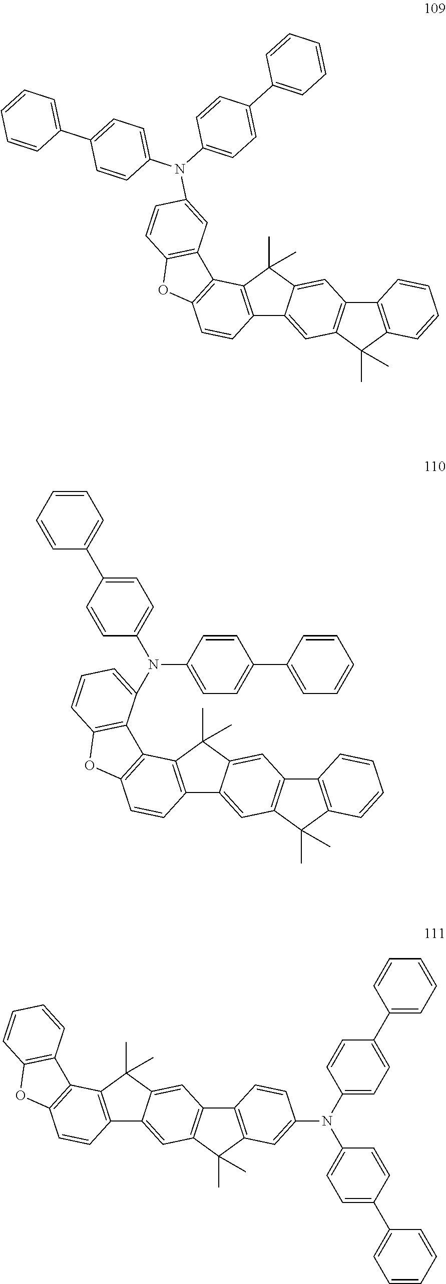 [SMILES: CC1(C)c2ccccc2-c2cc3c(cc21)-c1ccc2oc4ccc(N(c5ccc(-c6ccccc6)cc5)c5ccc(-c6ccccc6)cc5)cc4c2c1C3(C)C ; CC1(C)c2ccccc2-c2cc3c(cc21)-c1ccc2oc4cccc(N(c5ccc(-c6ccccc6)cc5)c5ccc(-c6ccccc6)cc5)c4c2c1C3(C)C ; CC1(C)c2cc(N(c3ccc(-c4ccccc4)cc3)c3ccc(-c4ccccc4)cc3)ccc2-c2cc3c(cc21)-c1ccc2oc4ccccc4c2c1C3(C)C]

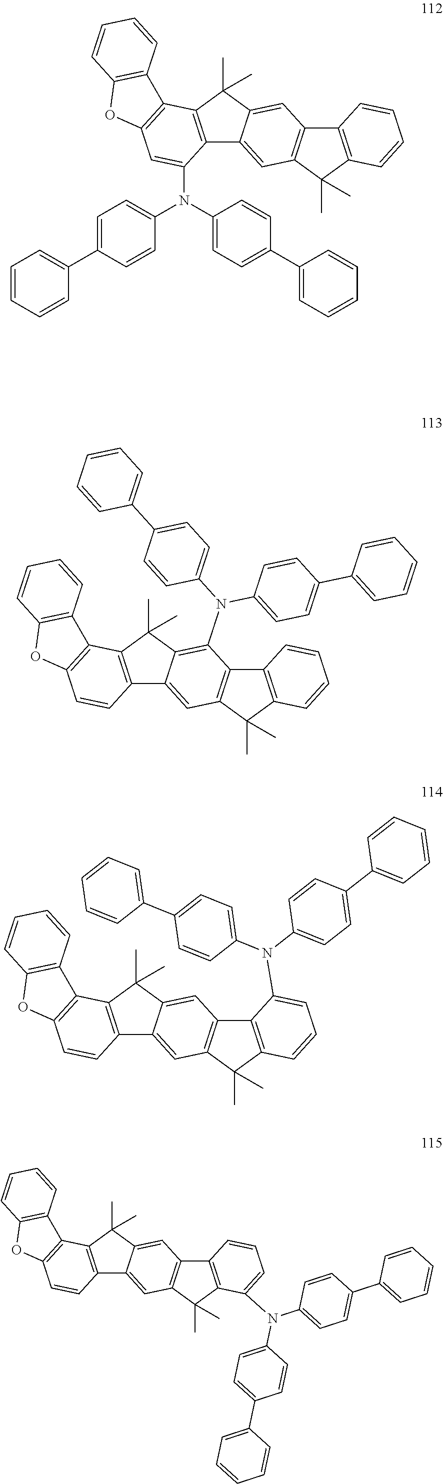 [SMILES: CC1(C)c2ccccc2-c2cc3c(cc21)-c1c(N(c2ccc(-c4ccccc4)cc2)c2ccc(-c4ccccc4)cc2)cc2oc4ccccc4c2c1C3(C)C ; CC1(C)c2ccccc2-c2c1cc1c(c2N(c2ccc(-c3ccccc3)cc2)c2ccc(-c3ccccc3)cc2)C(C)(C)c2c-1ccc1oc3ccccc3c21 ; CC1(C)c2cc3c(cc2-c2c(N(c4ccc(-c5ccccc5)cc4)c4ccc(-c5ccccc5)cc4)cccc21)C(C)(C)c1c-3ccc2oc3ccccc3c12 ; CC1(C)c2cc3c(cc2-c2cccc(N(c4ccc(-c5ccccc5)cc4)c4ccc(-c5ccccc5)cc4)c21)C(C)(C)c1c-3ccc2oc3ccccc3c12]

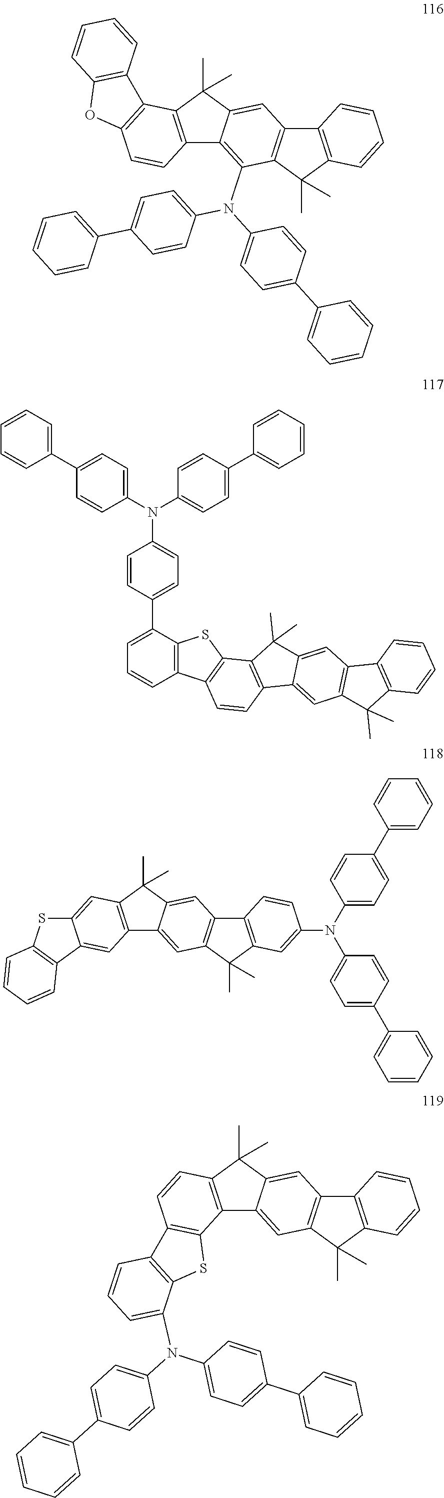 [SMILES: CC1(C)c2ccccc2-c2cc3c(c(N(c4ccc(-c5ccccc5)cc4)c4ccc(-c5ccccc5)cc4)c21)-c1ccc2oc4ccccc4c2c1C3(C)C ; CC1(C)c2ccccc2-c2cc3c(cc21)-c1ccc2c(sc4c(-c5ccc(N(c6ccc(-c7ccccc7)cc6)c6ccc(-c7ccccc7)cc6)cc5)cccc42)c1C3(C)C ; CC1(C)c2cc(N(c3ccc(-c4ccccc4)cc3)c3ccc(-c4ccccc4)cc3)ccc2-c2cc3c(cc21)-c1cc2c(cc1C3(C)C)sc1ccccc12 ; CC1(C)c2ccccc2-c2cc3c(cc21)-c1c(ccc2c1sc1c(N(c4ccc(-c5ccccc5)cc4)c4ccc(-c5ccccc5)cc4)cccc12)C3(C)C]

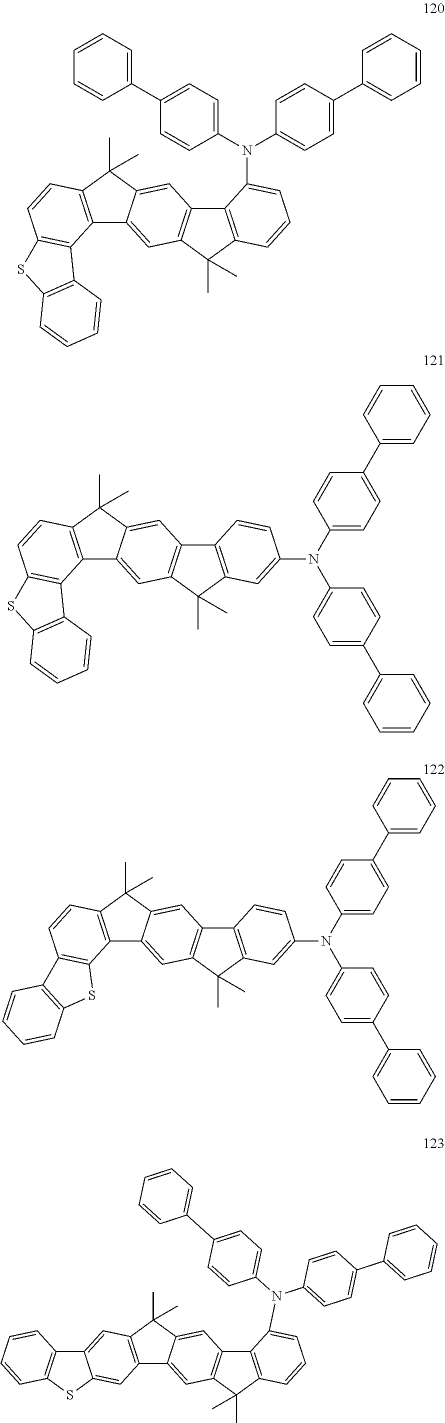 [SMILES: CC1(C)c2cc3c(cc2-c2c(N(c4ccc(-c5ccccc5)cc4)c4ccc(-c5ccccc5)cc4)cccc21)C(C)(C)c1ccc2sc4ccccc4c2c1-3 ; CC1(C)c2cc(N(c3ccc(-c4ccccc4)cc3)c3ccc(-c4ccccc4)cc3)ccc2-c2cc3c(cc21)-c1c(ccc2sc4ccccc4c12)C3(C)C ; CC1(C)c2cc(N(c3ccc(-c4ccccc4)cc3)c3ccc(-c4ccccc4)cc3)ccc2-c2cc3c(cc21)-c1c(ccc2c1sc1ccccc12)C3(C)C ; CC1(C)c2cc3c(cc2-c2cc4sc5ccccc5c4cc21)C(C)(C)c1cccc(N(c2ccc(-c4ccccc4)cc2)c2ccc(-c4ccccc4)cc2)c1-3]

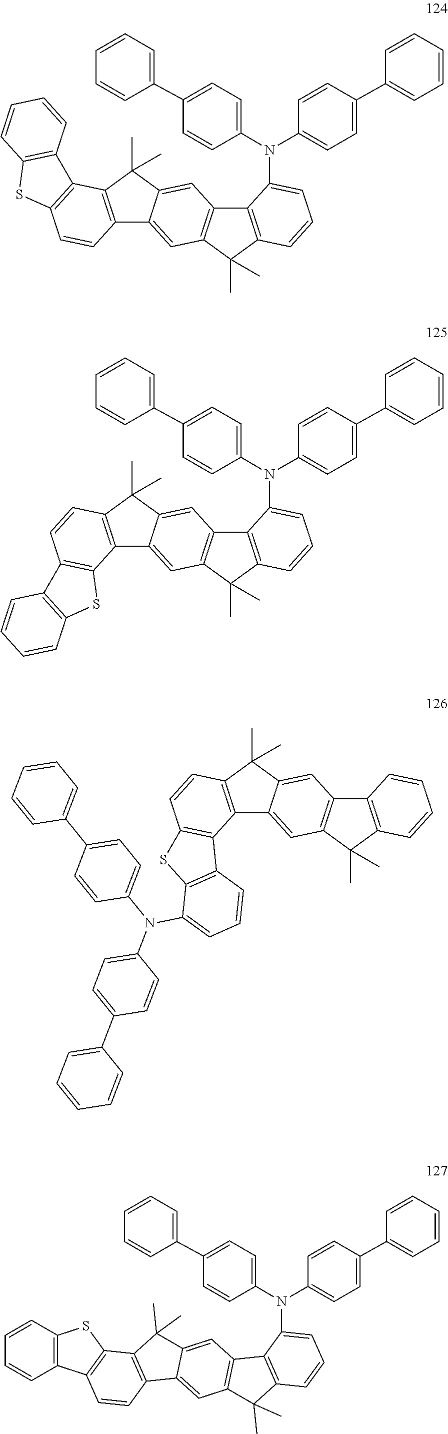 [SMILES: CC1(C)c2cc3c(cc2-c2c(N(c4ccc(-c5ccccc5)cc4)c4ccc(-c5ccccc5)cc4)cccc21)C(C)(C)c1c-3ccc2sc3ccccc3c12 ; CC1(C)c2cc3c(cc2-c2c(N(c4ccc(-c5ccccc5)cc4)c4ccc(-c5ccccc5)cc4)cccc21)C(C)(C)c1ccc2c(sc4ccccc42)c1-3 ; CC1(C)c2ccccc2-c2cc3c(cc21)-c1c(ccc2sc4c(N(c5ccc(-c6ccccc6)cc5)c5ccc(-c6ccccc6)cc5)cccc4c12)C3(C)C ; CC1(C)c2cc3c(cc2-c2c(N(c4ccc(-c5ccccc5)cc4)c4ccc(-c5ccccc5)cc4)cccc21)C(C)(C)c1c-3ccc2c1sc1ccccc12]

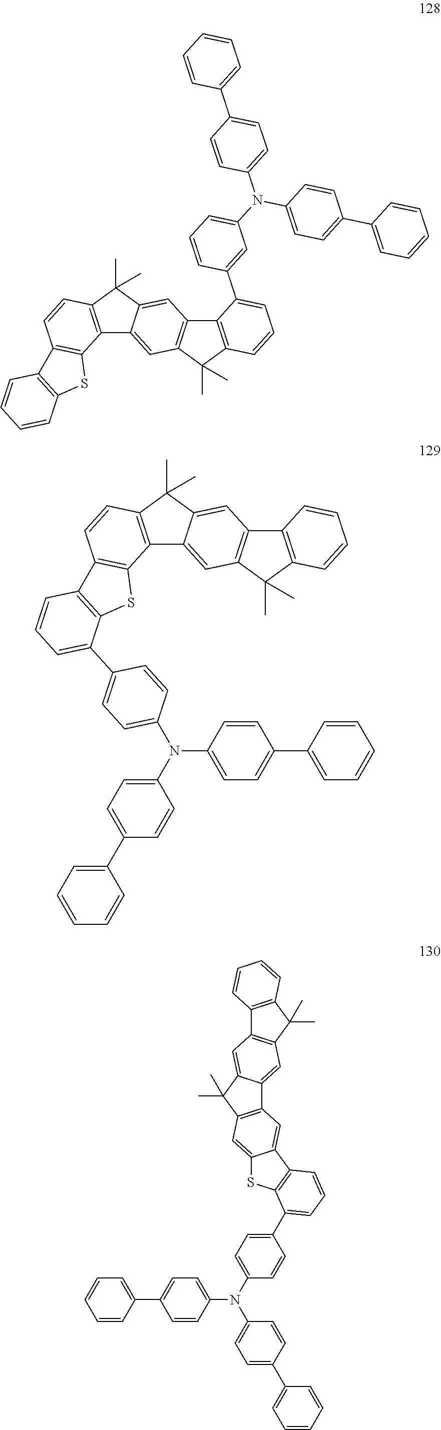 [SMILES: CC1(C)c2cc3c(cc2-c2c(-c4cccc(N(c5ccc(-c6ccccc6)cc5)c5ccc(-c6ccccc6)cc5)c4)cccc21)C(C)(C)c1ccc2c(sc4ccccc42)c1-3 ; CC1(C)c2ccccc2-c2cc3c(cc21)-c1c(ccc2c1sc1c(-c4ccc(N(c5ccc(-c6ccccc6)cc5)c5ccc(-c6ccccc6)cc5)cc4)cccc12)C3(C)C ; CC1(C)c2ccccc2-c2cc3c(cc21)-c1cc2c(cc1C3(C)C)sc1c(-c3ccc(N(c4ccc(-c5ccccc5)cc4)c4ccc(-c5ccccc5)cc4)cc3)cccc12]

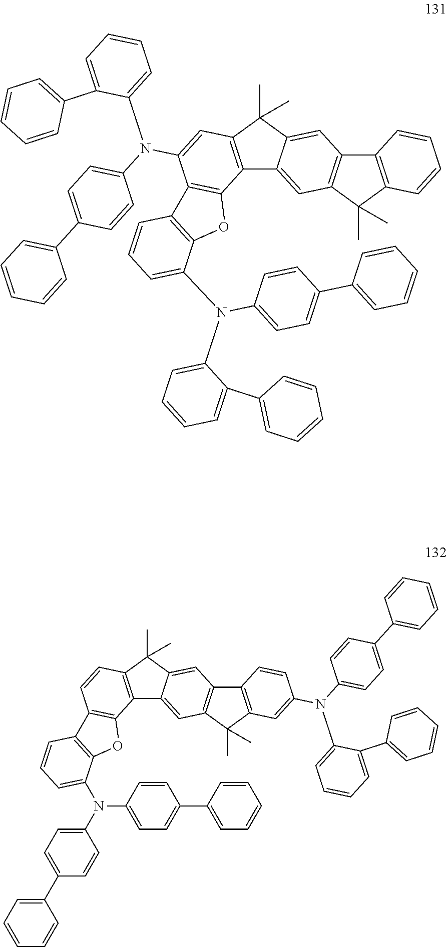 [SMILES: CC1(C)c2ccccc2-c2cc3c(cc21)-c1c(cc(N(c2ccc(-c4ccccc4)cc2)c2ccccc2-c2ccccc2)c2c1oc1c(N(c4ccc(-c5ccccc5)cc4)c4ccccc4-c4ccccc4)cccc12)C3(C)C ; CC1(C)c2cc(N(c3ccc(-c4ccccc4)cc3)c3ccccc3-c3ccccc3)ccc2-c2cc3c(cc21)-c1c(ccc2c1oc1c(N(c4ccc(-c5ccccc5)cc4)c4ccc(-c5ccccc5)cc4)cccc12)C3(C)C]

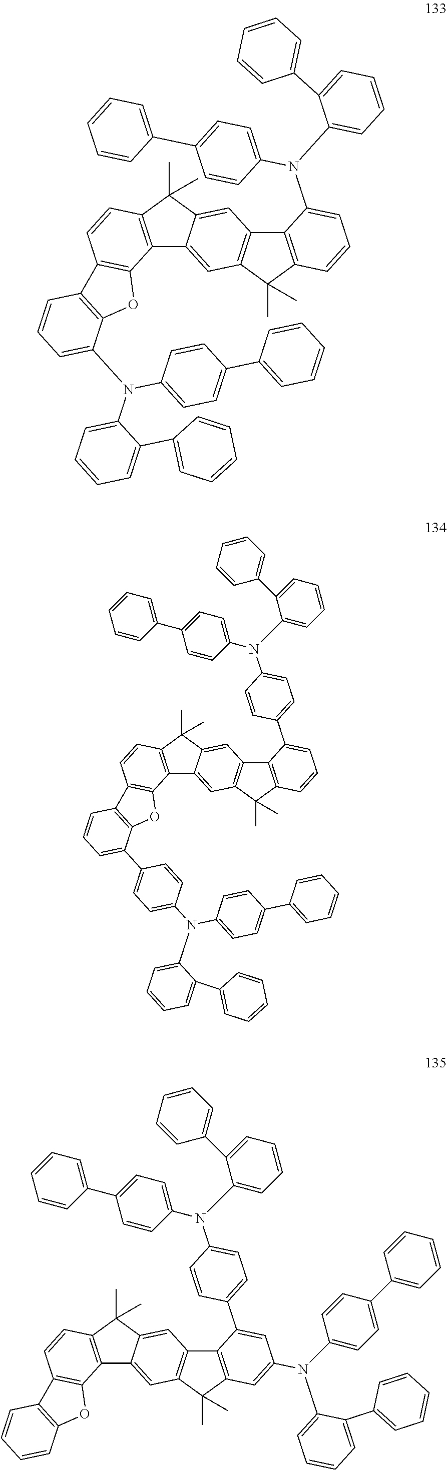 [SMILES: CC1(C)c2cc3c(cc2-c2c(N(c4ccc(-c5ccccc5)cc4)c4ccccc4-c4ccccc4)cccc21)C(C)(C)c1ccc2c(oc4c(N(c5ccc(-c6ccccc6)cc5)c5ccccc5-c5ccccc5)cccc42)c1-3 ; CC1(C)c2cc3c(cc2-c2c(-c4ccc(N(c5ccc(-c6ccccc6)cc5)c5ccccc5-c5ccccc5)cc4)cccc21)C(C)(C)c1ccc2c(oc4c(-c5ccc(N(c6ccc(-c7ccccc7)cc6)c6ccccc6-c6ccccc6)cc5)cccc42)c1-3 ; CC1(C)c2cc3c(cc2-c2c(-c4ccc(N(c5ccc(-c6ccccc6)cc5)c5ccccc5-c5ccccc5)cc4)cc(N(c4ccc(-c5ccccc5)cc4)c4ccccc4-c4ccccc4)cc21)C(C)(C)c1ccc2c(oc4ccccc42)c1-3]

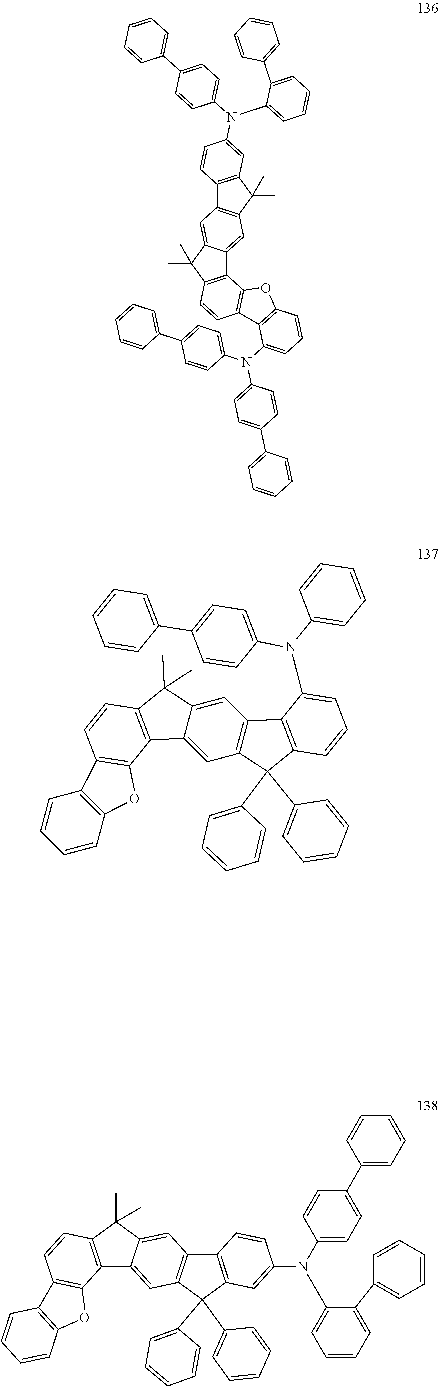 [SMILES: CC1(C)c2cc(N(c3ccc(-c4ccccc4)cc3)c3ccccc3-c3ccccc3)ccc2-c2cc3c(cc21)-c1c(ccc2c1oc1cccc(N(c4ccc(-c5ccccc5)cc4)c4ccc(-c5ccccc5)cc4)c12)C3(C)C ; CC1(C)c2cc3c(cc2-c2c1ccc1c2oc2ccccc21)C(c1ccccc1)(c1ccccc1)c1cccc(N(c2ccccc2)c2ccc(-c4ccccc4)cc2)c1-3 ; CC1(C)c2cc3c(cc2-c2c1ccc1c2oc2ccccc21)C(c1ccccc1)(c1ccccc1)c1cc(N(c2ccc(-c4ccccc4)cc2)c2ccccc2-c2ccccc2)ccc1-3]

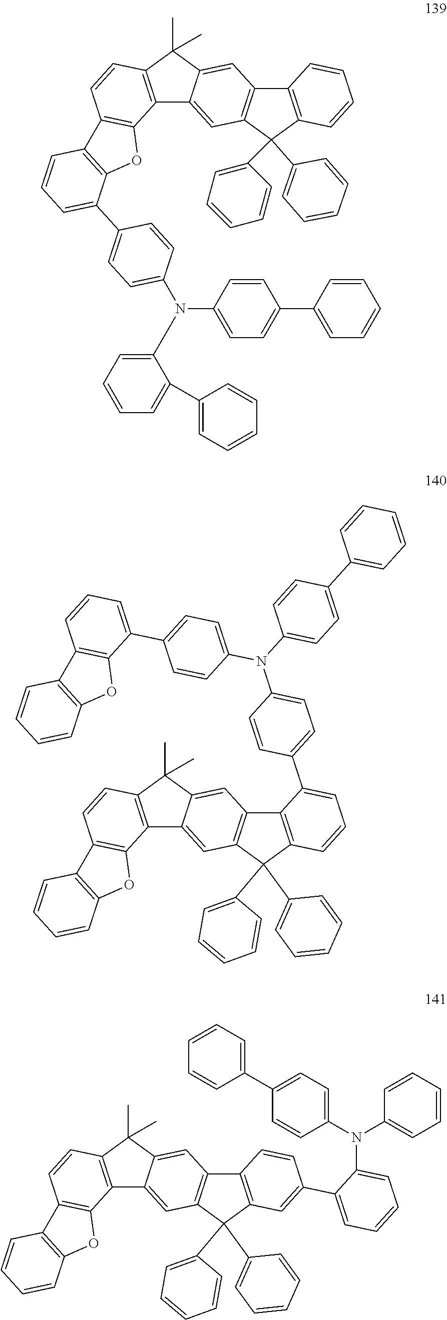 [SMILES: CC1(C)c2cc3c(cc2-c2c1ccc1c2oc2c(-c4ccc(N(c5ccc(-c6ccccc6)cc5)c5ccccc5-c5ccccc5)cc4)cccc21)C(c1ccccc1)(c1ccccc1)c1ccccc1-3 ; CC1(C)c2cc3c(cc2-c2c1ccc1c2oc2ccccc21)C(c1ccccc1)(c1ccccc1)c1cccc(-c2ccc(N(c4ccc(-c5ccccc5)cc4)c4ccc(-c5cccc6c5oc5ccccc56)cc4)cc2)c1-3 ; CC1(C)c2cc3c(cc2-c2c1ccc1c2oc2ccccc21)C(c1ccccc1)(c1ccccc1)c1cc(-c2ccccc2N(c2ccccc2)c2ccc(-c4ccccc4)cc2)ccc1-3]

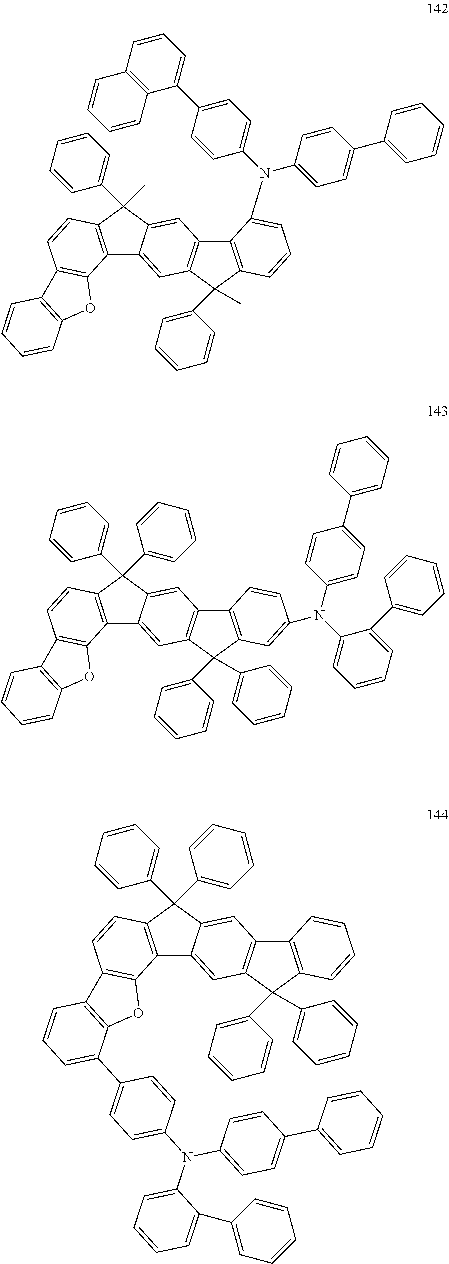 [SMILES: CC1(c2ccccc2)c2cc3c(cc2-c2c(N(c4ccc(-c5ccccc5)cc4)c4ccc(-c5cccc6ccccc56)cc4)cccc21)C(C)(c1ccccc1)c1ccc2c(oc4ccccc42)c1-3 ; c1ccc(-c2ccc(N(c3ccc4c(c3)C(c3ccccc3)(c3ccccc3)c3cc5c(cc3-4)C(c3ccccc3)(c3ccccc3)c3ccc4c(oc6ccccc64)c3-5)c3ccccc3-c3ccccc3)cc2)cc1 ; c1ccc(-c2ccc(N(c3ccc(-c4cccc5c4oc4c6c(ccc45)C(c4ccccc4)(c4ccccc4)c4cc5c(cc4-6)C(c4ccccc4)(c4ccccc4)c4ccccc4-5)cc3)c3ccccc3-c3ccccc3)cc2)cc1]

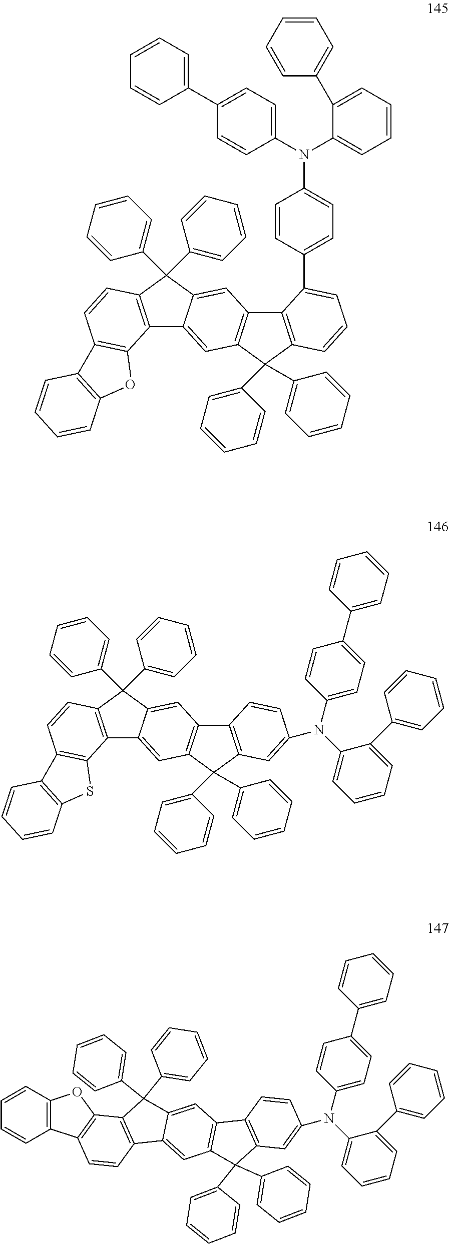 [SMILES: c1ccc(-c2ccc(N(c3ccc(-c4cccc5c4-c4cc6c(cc4C5(c4ccccc4)c4ccccc4)-c4c(ccc5c4oc4ccccc45)C6(c4ccccc4)c4ccccc4)cc3)c3ccccc3-c3ccccc3)cc2)cc1 ; c1ccc(-c2ccc(N(c3ccc4c(c3)C(c3ccccc3)(c3ccccc3)c3cc5c(cc3-4)C(c3ccccc3)(c3ccccc3)c3ccc4c(sc6ccccc64)c3-5)c3ccccc3-c3ccccc3)cc2)cc1 ; c1ccc(-c2ccc(N(c3ccc4c(c3)C(c3ccccc3)(c3ccccc3)c3cc5c(cc3-4)C(c3ccccc3)(c3ccccc3)c3c-5ccc4c3oc3ccccc34)c3ccccc3-c3ccccc3)cc2)cc1]

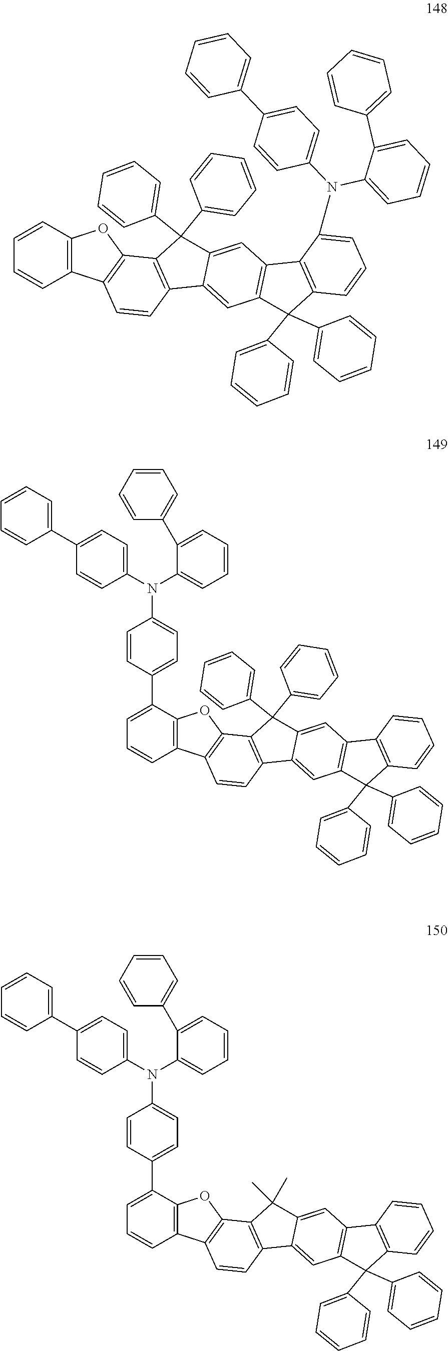 [SMILES: c1ccc(-c2ccc(N(c3ccccc3-c3ccccc3)c3cccc4c3-c3cc5c(cc3C4(c3ccccc3)c3ccccc3)-c3ccc4c(oc6ccccc64)c3C5(c3ccccc3)c3ccccc3)cc2)cc1 ; c1ccc(-c2ccc(N(c3ccc(-c4cccc5c4oc4c6c(ccc45)-c4cc5c(cc4C6(c4ccccc4)c4ccccc4)-c4ccccc4C5(c4ccccc4)c4ccccc4)cc3)c3ccccc3-c3ccccc3)cc2)cc1 ; CC1(C)c2cc3c(cc2-c2ccc4c(oc5c(-c6ccc(N(c7ccc(-c8ccccc8)cc7)c7ccccc7-c7ccccc7)cc6)cccc54)c21)C(c1ccccc1)(c1ccccc1)c1ccccc1-3]

The compounds of formula (I) may be prepared by the synthesis method described hereinafter. The person skilled in the art will be able to modify these within the scope of their knowledge in the art in order to prepare further compounds according to the application that are not directly comparable by the methods shown below.

The compounds of the formula (I) may be produced by different synthesis routes that are elucidated in detail hereinafter.

In a first variant (scheme 1), a benzene derivative having two carboxylic ester groups, one dibenzofuran or dibenzothiophene group and one halogen atom as substituent is coupled to a benzene derivative having an amino group and a boronic acid as substituent in a Suzuki reaction (a)). Thereafter, metal organyl is added onto the carbonyl group of the carboxylic ester groups, preferably magnesium organyl in a Grignard reaction (b)). This forms tertiary alcohol groups on the benzene rings. These are cyclized in a further step under acidic conditions, so as to form the indenofluorenyl structure (c)).

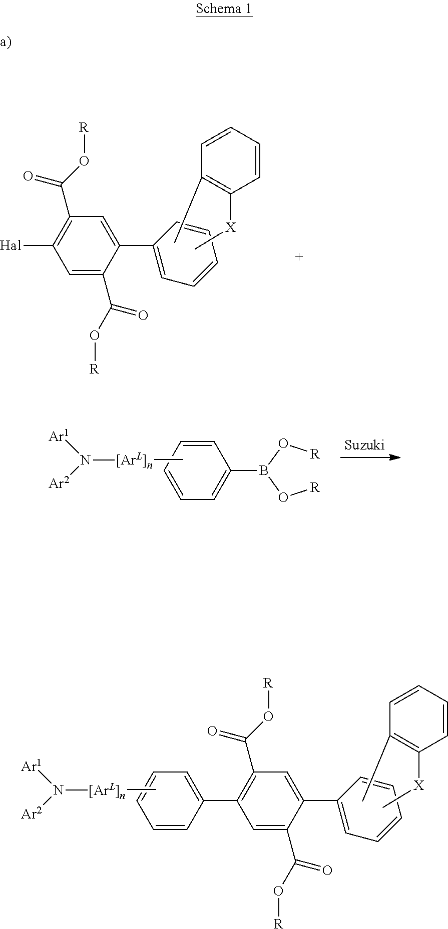

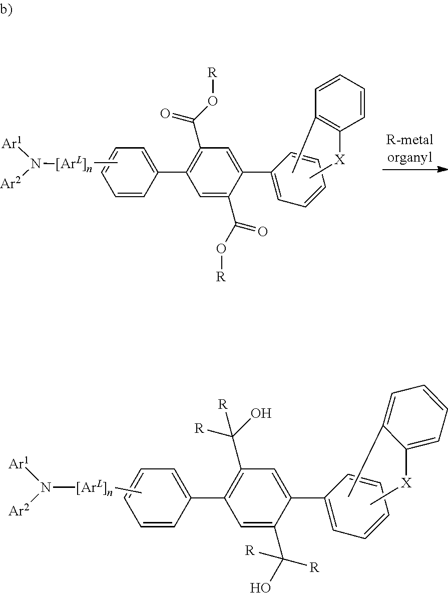

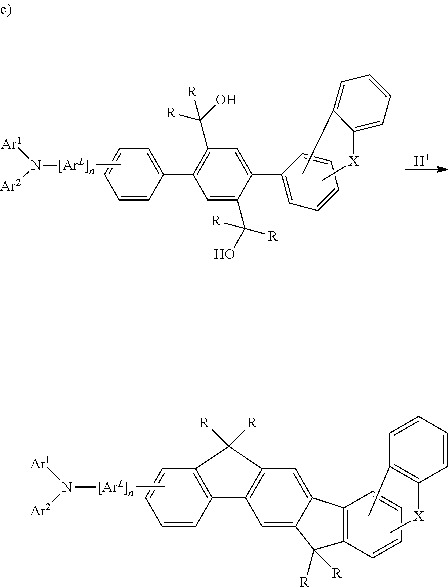

X is O or S, Ar^{L} and n, and Ar¹ and Ar² are as defined above. Hal is Cl, Br or I, preferably Cl. R is an organic radical.

In a second variant (scheme 2), steps a) to c) are conducted as shown above in scheme 1, except that, rather than amine-substituted phenyl, halogen-substituted phenyl is used in step a). Subsequently, in a step d), a Suzuki coupling reaction is conducted with an Ar¹Ar²N—Ar^{L}—B(OR)₂ group, or a Buchwald coupling reaction with an Ar¹Ar²NH group (d)).

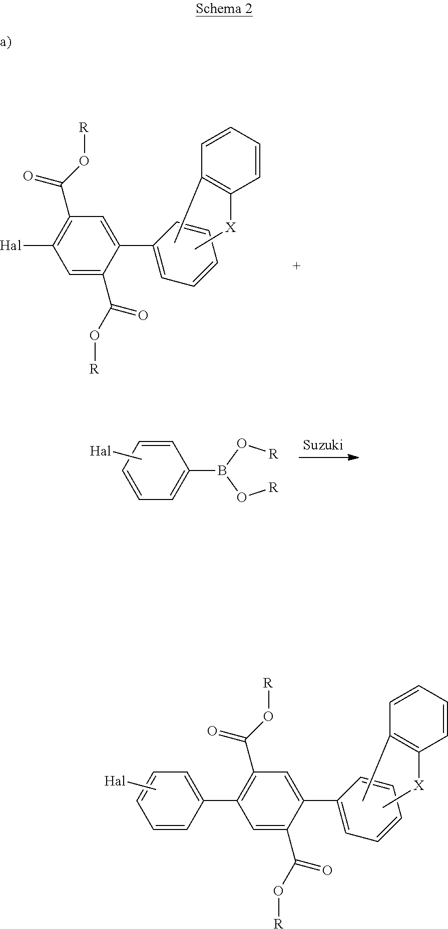

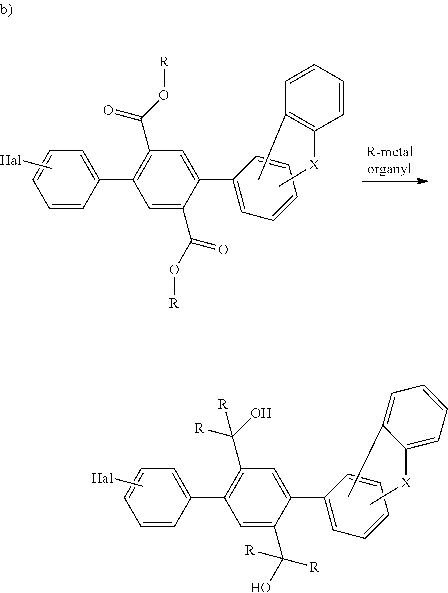

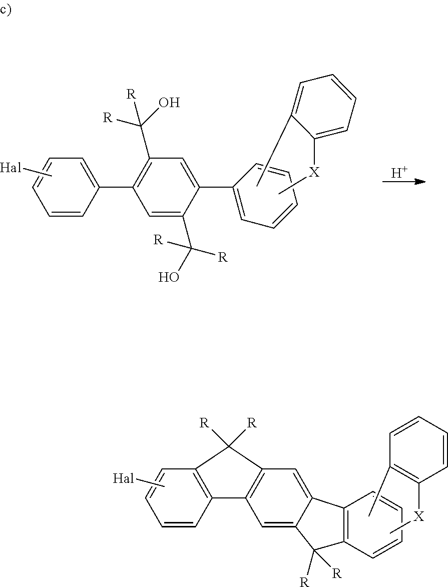

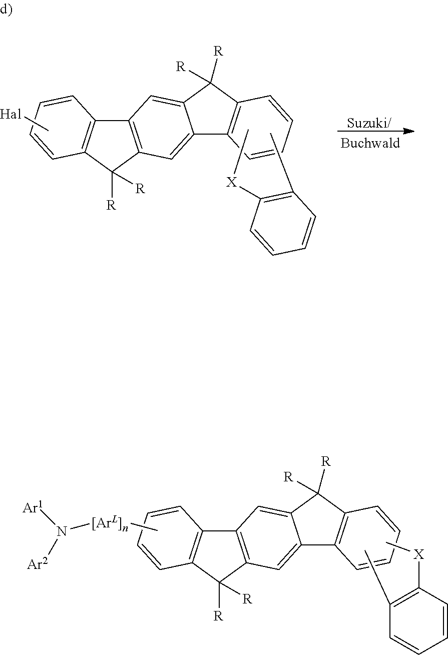

X is O or S, Ar^{L} and n, and Ar¹ and Ar² are as defined above. Hal is Cl, Br or I, preferably Cl. R is an organic radical.

In a third variant (scheme 3), steps a) to c) are conducted as shown above in scheme 1, except that, rather than amine-substituted phenyl, unsubstituted phenyl is used in step a). After step c), a borylation (d)) is conducted, followed by a Suzuki coupling with a compound Ar¹Ar²N—Ar^{L}-Hal (e)).

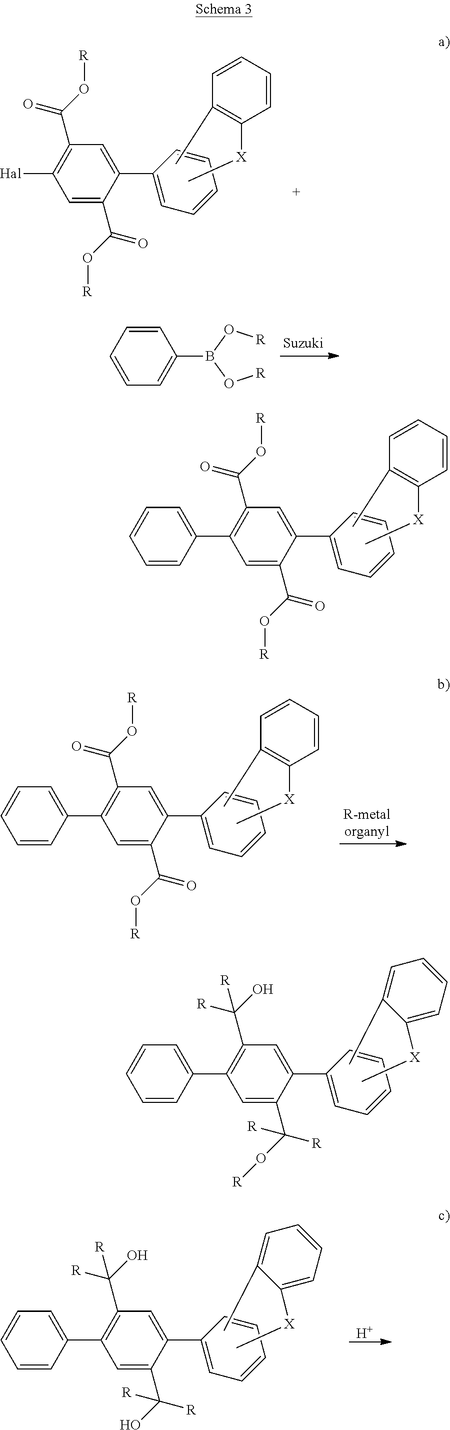

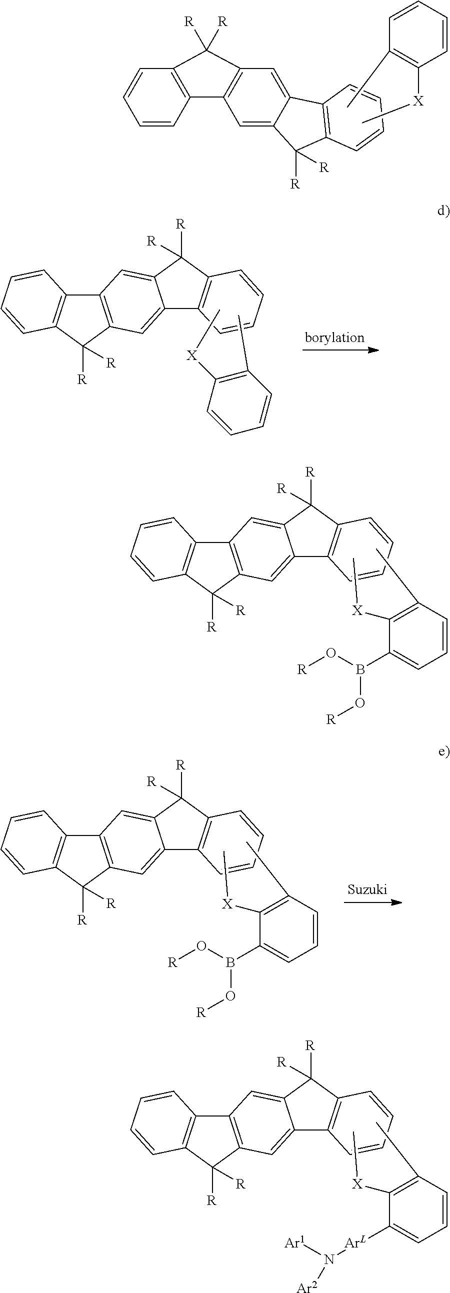

X, Ar^{L} and n, and Ar¹ and Ar², are as defined above. Hal is Cl, Br or I, preferably Cl. R is an organic radical.

For preparation of compounds that correspond to the preferred embodiments of formula (I), according to one of formulae (I-3), (I-3-A), (I-3-1), (I-3-2), (I-3-1-A) and (I-3-2-A), in which the amino group is bonded to the middle benzene ring of the indenofluorene, the procedure for the synthesis is preferably in accordance with an alternative variant 4 (scheme 4). This proceeds from a fluorenyl derivative bearing two halogen atoms on one of its benzene rings. This is reacted (step a)) in a Suzuki or Buchwald reaction with a compound Ar¹Ar²N—Ar^{L}—B(OR)₂ or Ar¹Ar²NH, and then a boronic acid group is introduced. Thereafter, in a step b), a Suzuki reaction with a dibenzothiophene or dibenzofuran derivative bearing a carboxylic ester group is conducted on the boronic acid group. To the latter is added a metal organyl, as in steps b) and c) of schemes 1 to 3, and an acid-catalyzed ring closure reaction is conducted, in which the compound of the formula (I) is obtained (steps c) and d)).

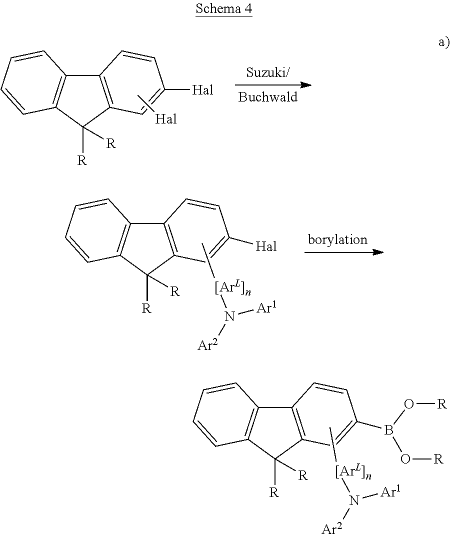

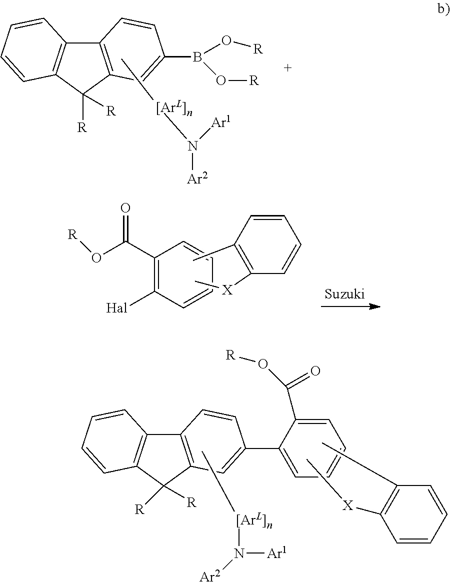

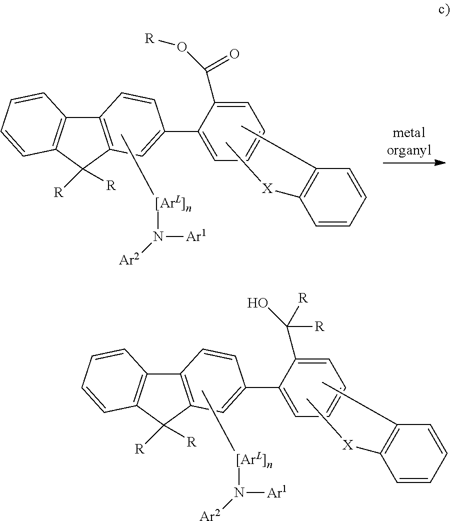

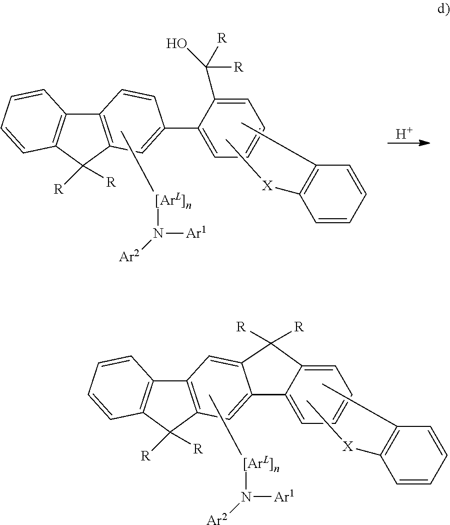

X, Ar^{L} and n, and Ar¹ and Ar², are as defined above. Hal is Cl, Br or I, preferably Cl. R is an organic radical.

The person skilled in the art will be able to make use of the processes described above to obtain compounds of the formula (I). They will alternatively also adapt and modify and optimize the processes within the scope of their common art knowledge, to the extent necessary, in order to prepare compounds of formula (I). The compounds shown above may each be substituted by organic radicals at their positions shown as unsubstituted.

The present application thus provides a process for preparing a compound of the formula (I), characterized in that there is at least one sequence of process steps a) followed by b), where process step a) comprises a reaction in which a carboxylic ester group that binds to a biphenyl unit in the ortho position to the bond between the two benzene rings of the biphenyl unit is converted to a tertiary alcohol group by addition of a metal organyl, preferably Grignard reagent, and where process step b) comprises a reaction in which the tertiary alcohol group obtained in process step a) enters into a ring closure reaction under acidic conditions, especially acidic ion exchange resin, for example Amberlyst-15, such that a fluorenyl unit is formed from the biphenyl unit.

The process preferably encompasses further steps selected from Suzuki coupling, Buchwald coupling and borylation.

The above-described compounds of formula (I), especially compounds substituted by reactive leaving groups, such as bromine, iodine, chlorine, boronic acid or boronic esters, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C═C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired positions substituted by R¹, R², R³, or R⁴ in formula (I). According to the linkage of the compound of formula (I), the compound is part of a side chain of the oligomer or polymer or part of the main chain.

Further disclosure relating to oligomers, polymers or dendrimers can be found at page 49 line 26-page 51 line 17 of WO 2020/109434 A1. The disclosure of these text passages is hereby fully incorporated into the present application by citation.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (-)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound of formula (I) or at least one polymer, oligomer or dendrimer containing at least one unit of formula (I) and at least one solvent, preferably an organic solvent. The way in which such solutions can be prepared is known to those skilled in the art.

There follows a description of the use of the compound of the formula (I) according to the application:

The compound of formula (I) is suitable for use in an electronic device, especially an organic electroluminescent device (OLED). Depending on the substitution, the compound of the formula (I) can be used in different functions and layers. Preference is given to use as a hole-transporting material in a hole-transporting layer and/or as matrix material in an emitting layer, more preferably in combination with a phosphorescent emitter.

The invention therefore further provides for the use of a compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (0-lasers) and more preferably organic electroluminescent devices (OLEDs).

The invention further provides an electronic device comprising at least one compound of formula (I). This electronic device is preferably selected from the abovementioned devices.

Particular preference is given to an organic electroluminescent device comprising an anode, cathode and at least one emitting layer, characterized in that at least one organic layer comprising at least one compound of formula (I) is present in the device. Preference is given to an organic electroluminescent device comprising an anode, cathode and at least one emitting layer, characterized in that at least one organic layer in the device, selected from hole-transporting and emitting layers, comprises at least one compound of formula (I).

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
anode-
hole injection layer-
hole transport layer-
optionally further hole transport layers-
emitting layer-
optionally hole blocker layer-
electron transport layer-
electron injection layer-
cathode-.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of color-emitting emitter compounds, an emitter compound used individually which emits over a broad wavelength range may also be suitable.

It is preferable that the compound of the formula (I) is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound of the formula (I) contains a phosphorescent emitting layer, it is preferable that this layer contains two or more, preferably exactly two, different matrix materials (mixed matrix system). Preferred embodiments of mixed matrix systems are described in detail further down.

If the compound of formula (I) is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer comprising the compound of the formula (I) additionally comprises one or more further hole-transporting compounds. These further hole-transporting compounds are preferably selected from triarylamine compounds, more preferably from monotriarylamine compounds.

They are most preferably selected from the preferred embodiments of hole transport materials that are specified further down. In the preferred embodiment described, the compound of the formula (I) and the one or more further hole-transporting compounds are preferably each present in a proportion of at least 10%, more preferably each in a proportion of at least 20%.

In a preferred embodiment, a hole-transporting layer comprising the compound of the formula (I) additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, 12, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth(III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are also the compounds depicted explicitly on pages 86-87 of published specification WO2021/156323A1.

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I) may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

The compound of the formula (I) is preferably present in an electron blocker layer. In this case, it is preferably not p-doped. Further preferably, in this case, it is preferably in the form of a single compound in the layer without addition of a further compound.

In an alternative preferred embodiment, the compound of the formula (I) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (I) in a mixed matrix system is preferably the matrix material having hole-transporting properties. Correspondingly, when the compound of the formula (I) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

In a preferred embodiment, in the case of mixed matrix systems, the two or more matrix materials present in the mixed matrix system, at least one of which preferably conforms to the formula (I), are used as a mixture and applied by evaporation.

The desired electron-transporting and hole-transporting properties of the mixed matrix components may, however, also be combined mainly or entirely in a single mixed matrix component, in which case the further mixed matrix component(s) fulfill(s) other functions.

Preference is given to using the following material classes in the abovementioned layers of the device:

### Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

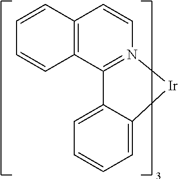

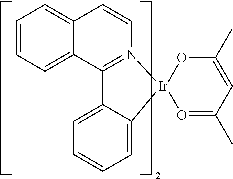

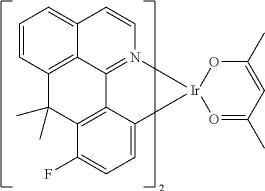

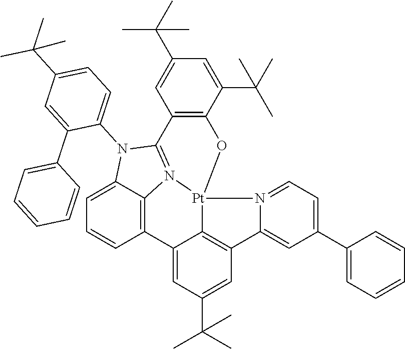

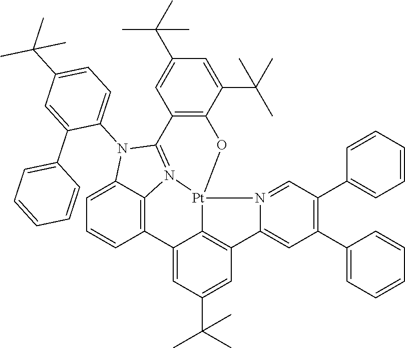

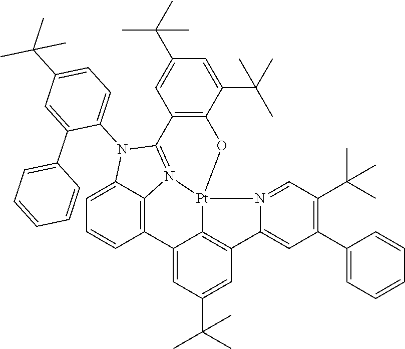

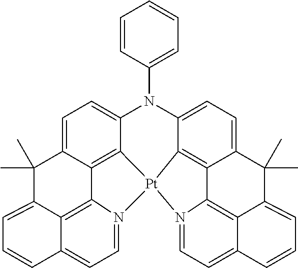

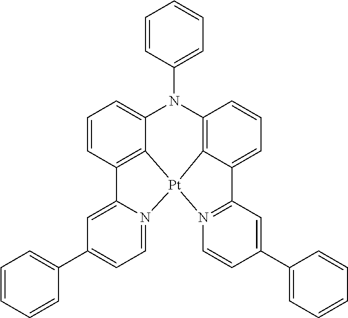

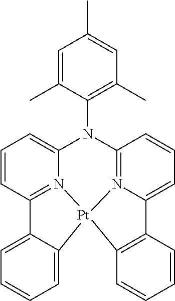

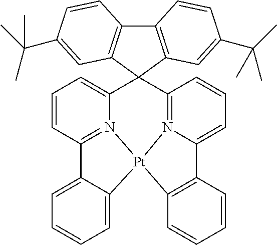

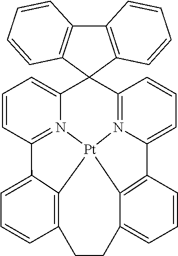

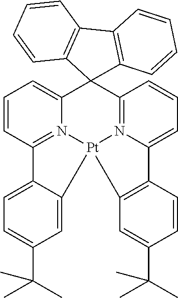

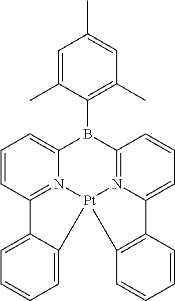

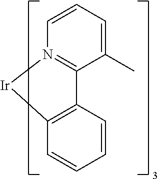

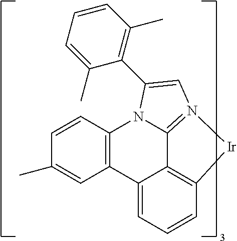

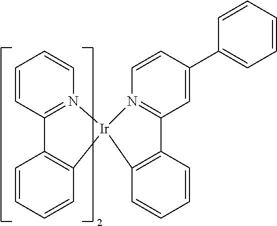

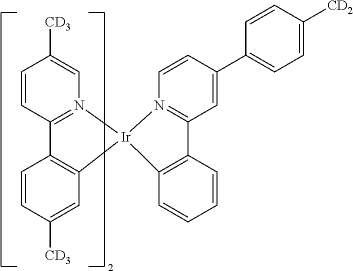

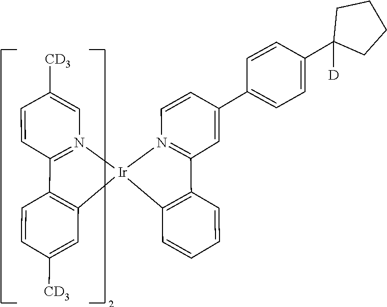

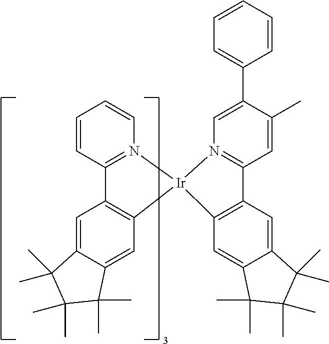

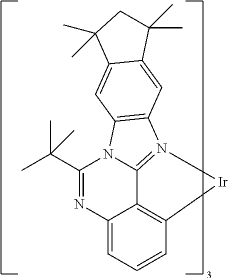

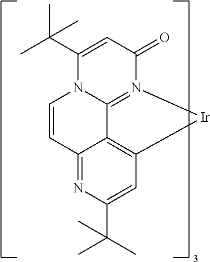

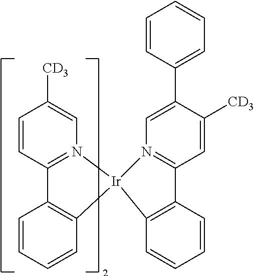

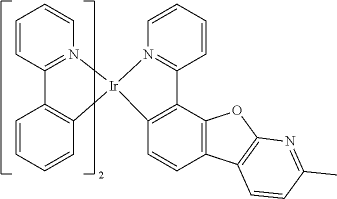

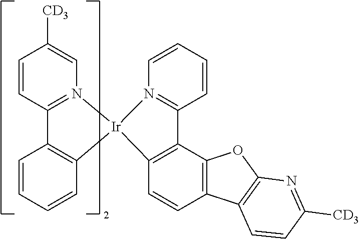

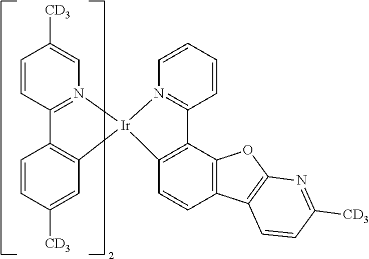

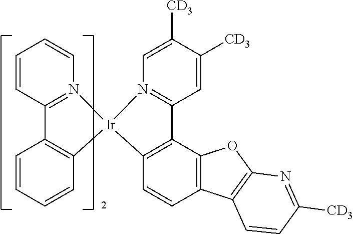

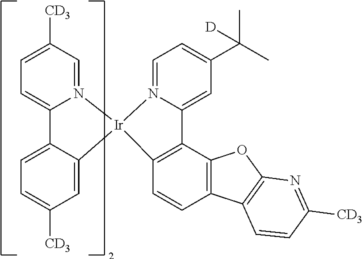

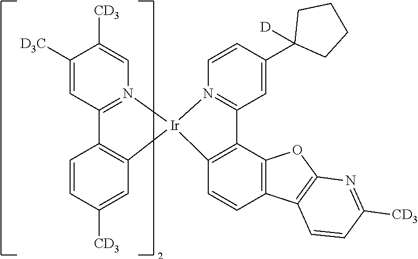

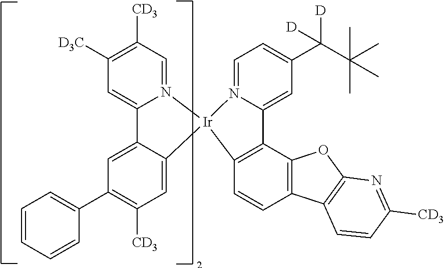

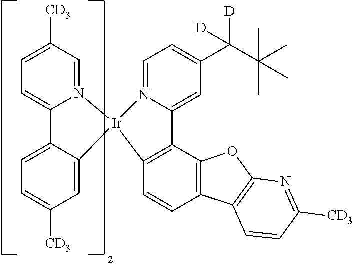

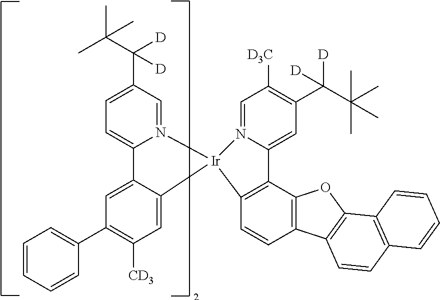

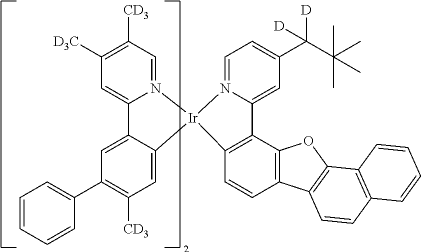

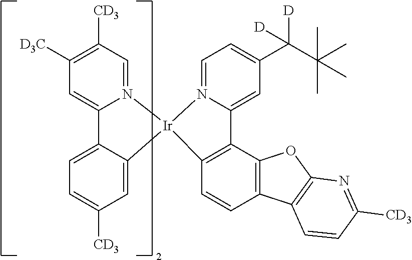

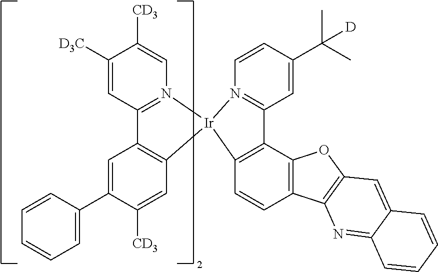

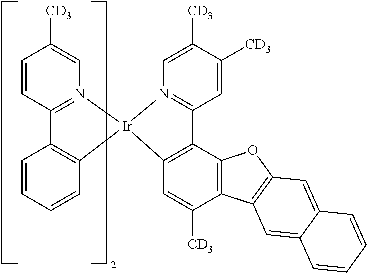

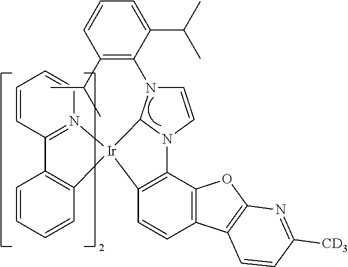

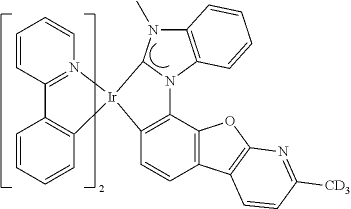

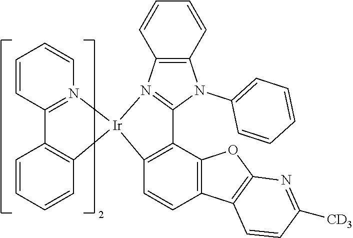

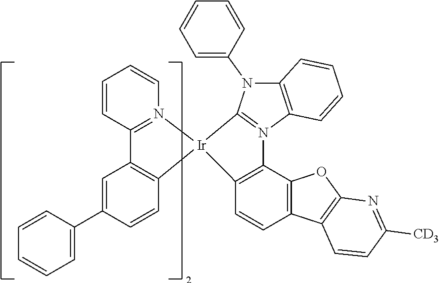

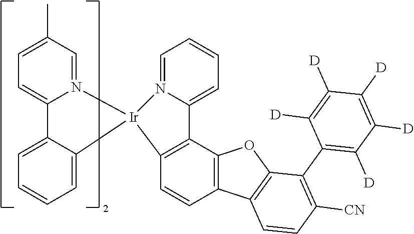

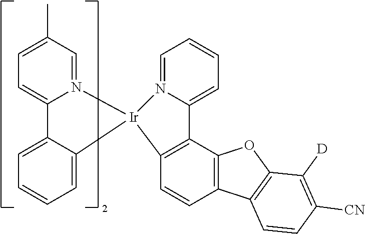

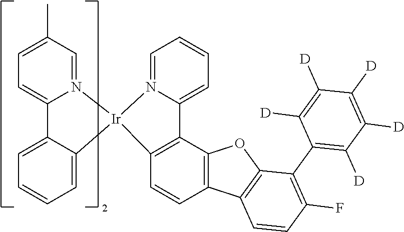

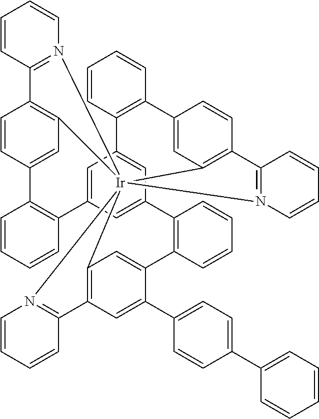

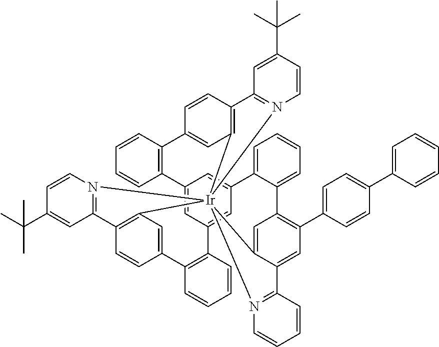

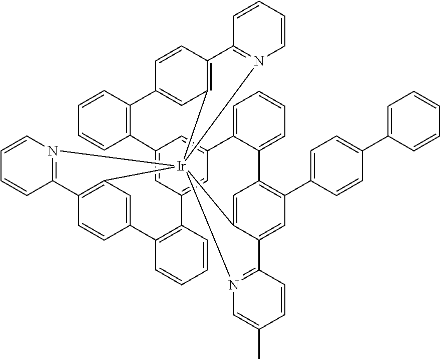

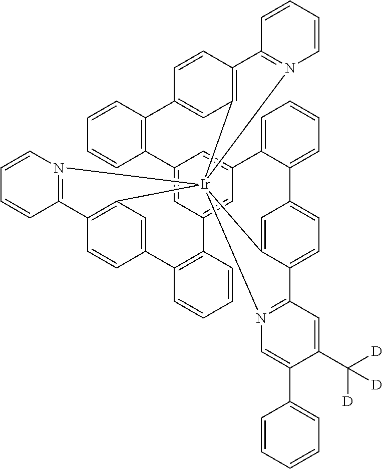

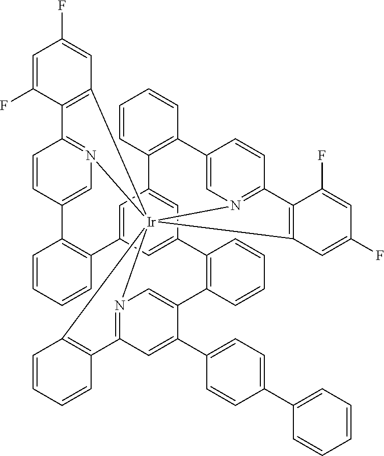

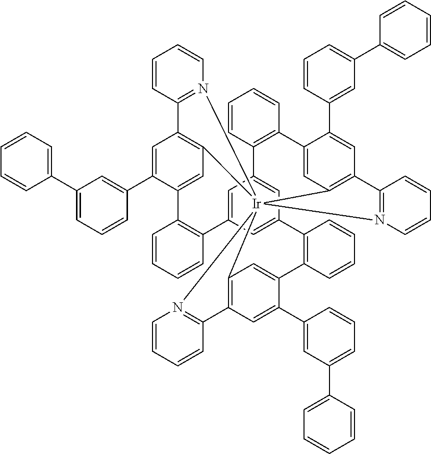

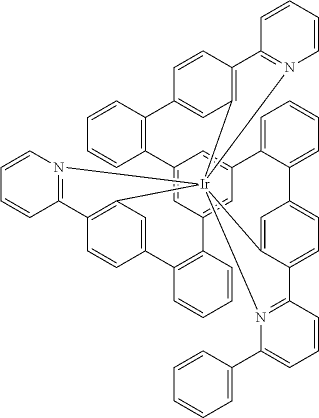

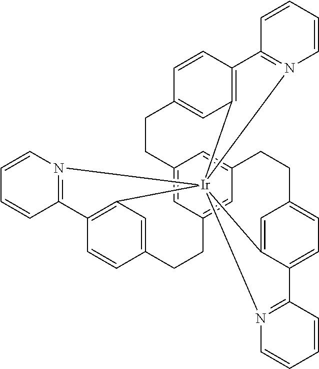

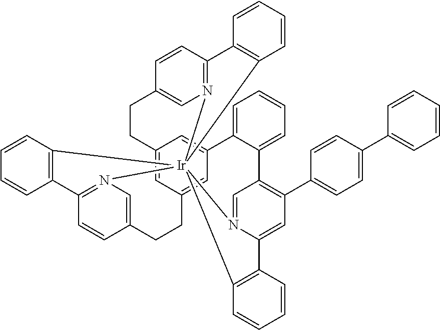

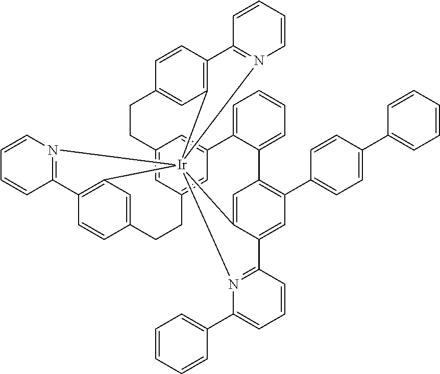

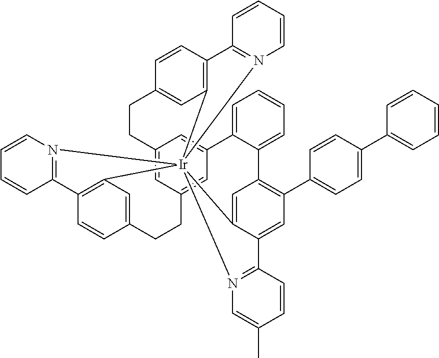

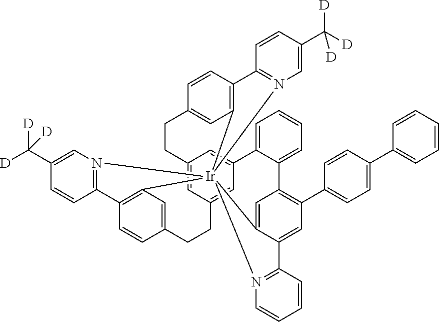

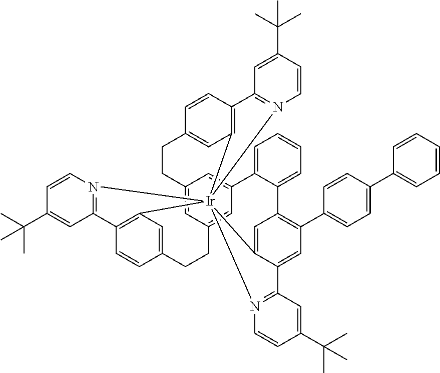

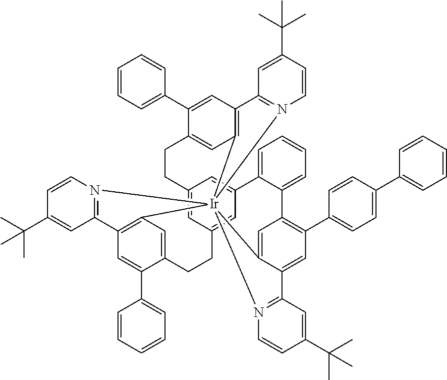

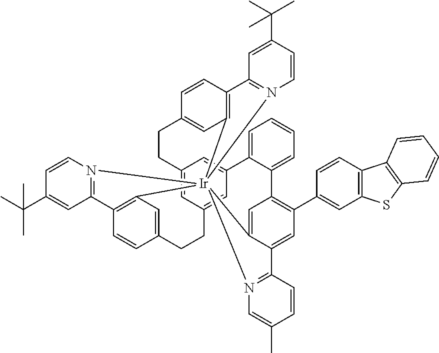

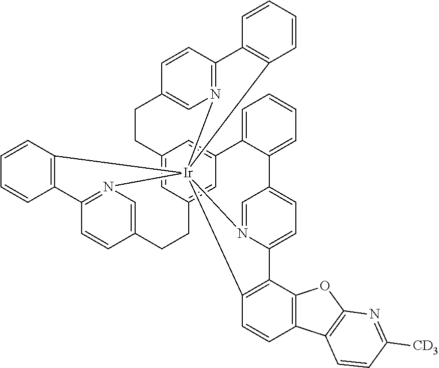

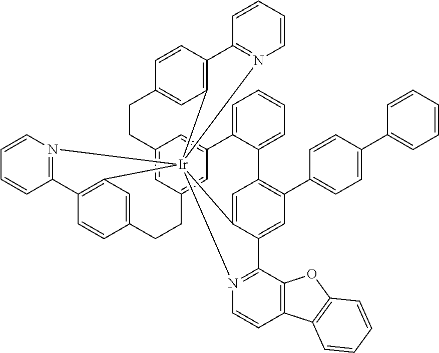

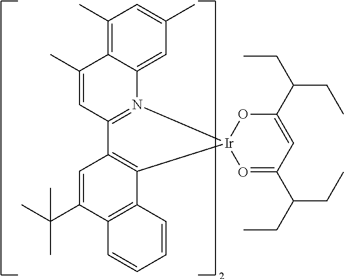

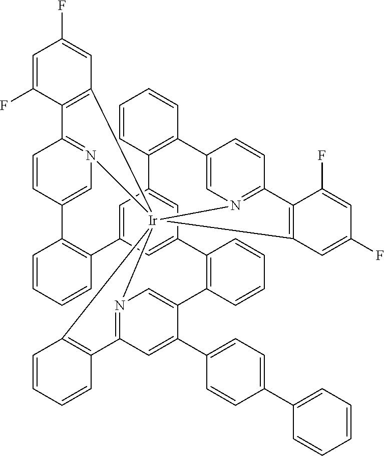

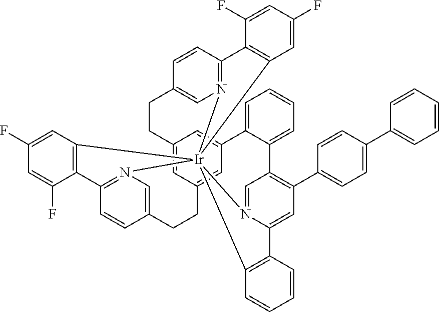

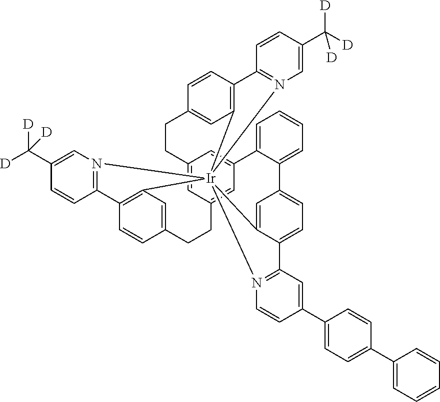

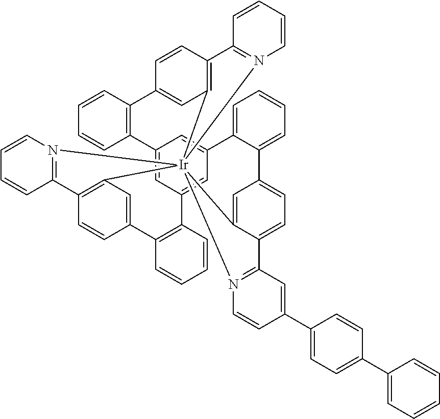

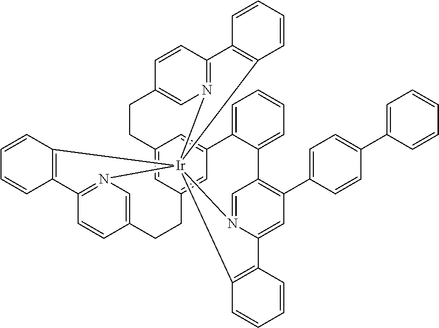

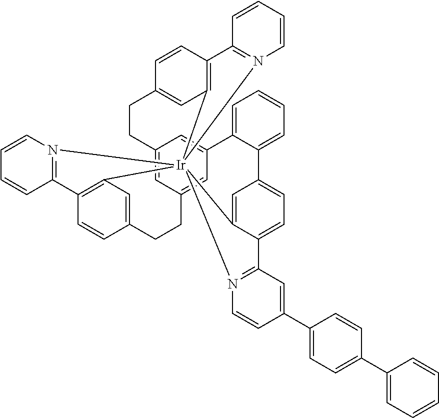

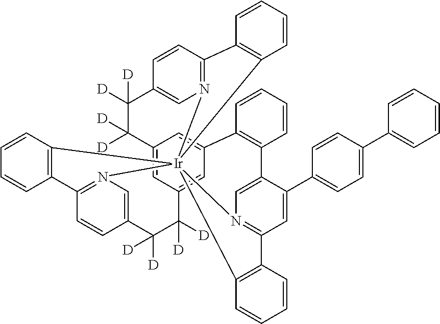

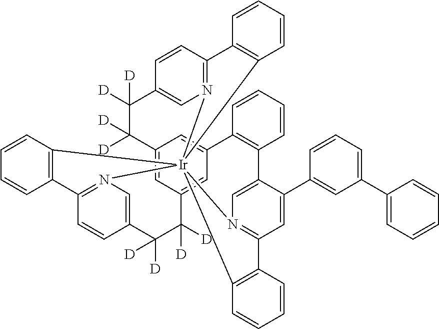

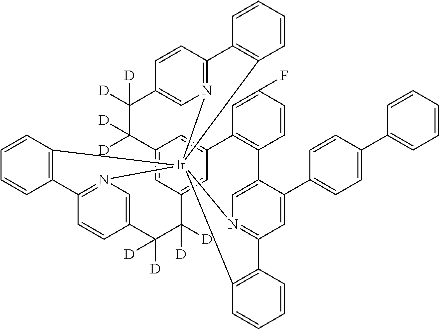

### Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. What is meant by an aromatic anthraceneamine is a compound in which a diarylamino group is bonded directly to an anthracene group, preferably in the 9 position. What is meant by an aromatic anthracenediamine is a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

### Matrix Materials for Fluorescent Emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

### Matrix Materials for Phosphorescent Emitters:

Preferred matrix materials for phosphorescent emitters are, as well as the compounds of the formula (I), aromatic ketones, aromatic phosphine oxides or aromatic sulfoxides or sulfones, triarylamines, carbazole derivatives, e.g. CBP (N,N-biscarbazolylbiphenyl) or carbazole derivatives, indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

### Electron-Transporting Materials:

Suitable electron-transporting materials are, for example, the compounds disclosed in Y. Shirota et al., Chem. Rev. 2007, 107(4), 953-1010, or other materials used in these layers according to the prior art.

Materials used for the electron transport layer may be any materials that are used as electron transport materials in the electron transport layer according to the prior art. Especially suitable are aluminum complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Preferred electron transport and electron injection materials are also the compounds shown explicitly on pages 73-75 of WO2020/109434A1.

### Hole-Transporting Materials:

Further compounds which, in addition to the compounds of the formula (I), are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups.

Preferred hole-transporting compounds are also the compounds depicted explicitly on pages 76-80 of WO2020/109434A1.

The compounds HT-1 to HT-21 below have particularly good suitability for use in a layer having hole transport function in an OLED. This is true not only of OLEDs according to the definitions and claims of the present application but also of OLEDs in general:

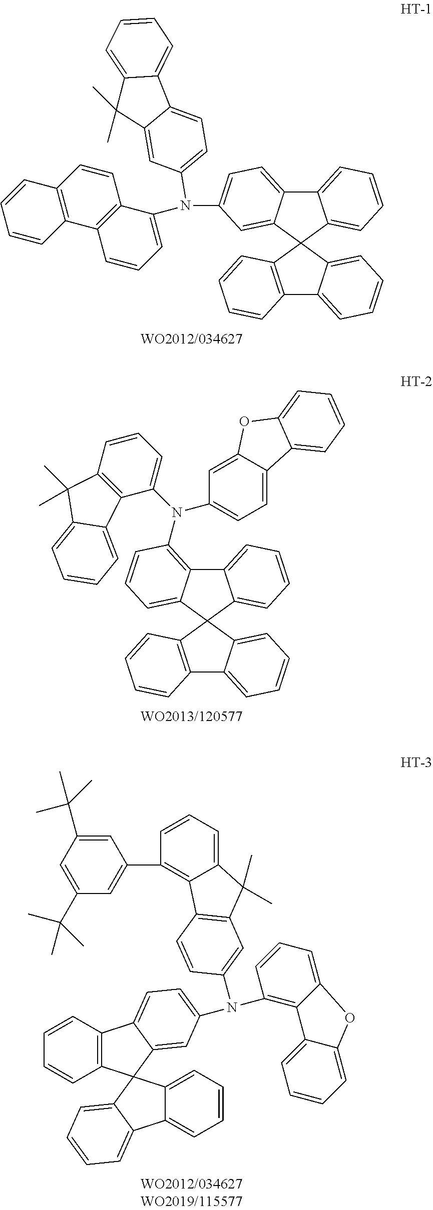 [SMILES: CC1(C)c2ccccc2-c2ccc(N(c3ccc4c(c3)C3(c5ccccc5-c5ccccc53)c3ccccc3-4)c3cccc4c3ccc3ccccc34)cc21 ; CC1(C)c2ccccc2-c2c(N(c3ccc4c(c3)oc3ccccc34)c3cccc4c3-c3ccccc3C43c4ccccc4-c4ccccc43)cccc21 ; CC(C)(C)c1cc(-c2cccc3c2-c2ccc(N(c4ccc5c(c4)C4(c6ccccc6-c6ccccc64)c4ccccc4-5)c4cccc5oc6ccccc6c45)cc2C3(C)C)cc(C(C)(C)C)c1]

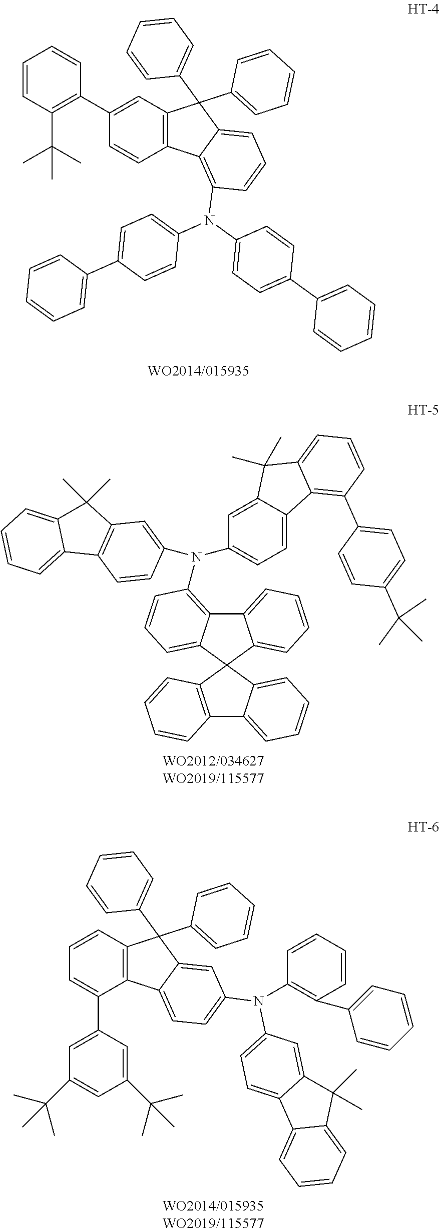

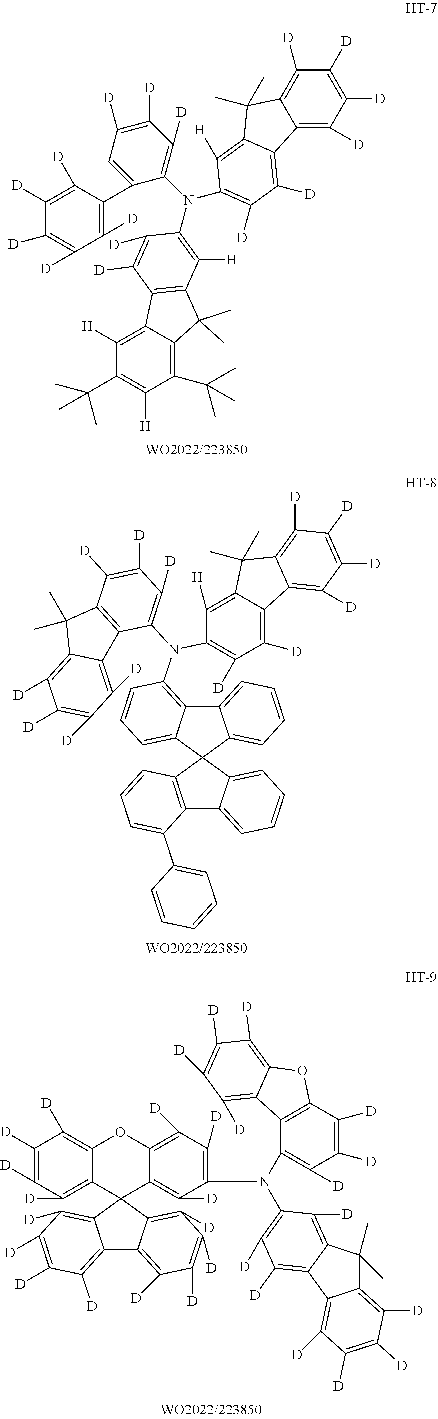

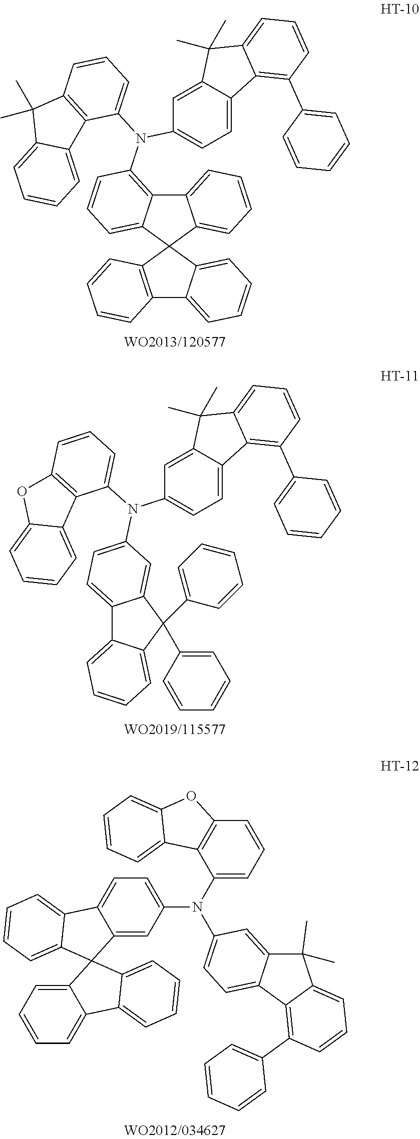 [SMILES: CC1(C)c2cc(N(c3cccc4c3-c3ccccc3C4(C)C)c3cccc4c3-c3ccccc3C43c4ccccc4-c4ccccc43)ccc2-c2c(-c3ccccc3)cccc21 ; CC1(C)c2cc(N(c3ccc4c(c3)C(c3ccccc3)(c3ccccc3)c3ccccc3-4)c3cccc4oc5ccccc5c34)ccc2-c2c(-c3ccccc3)cccc21 ; CC1(C)c2cc(N(c3ccc4c(c3)C3(c5ccccc5-c5ccccc53)c3ccccc3-4)c3cccc4oc5ccccc5c34)ccc2-c2c(-c3ccccc3)cccc21]

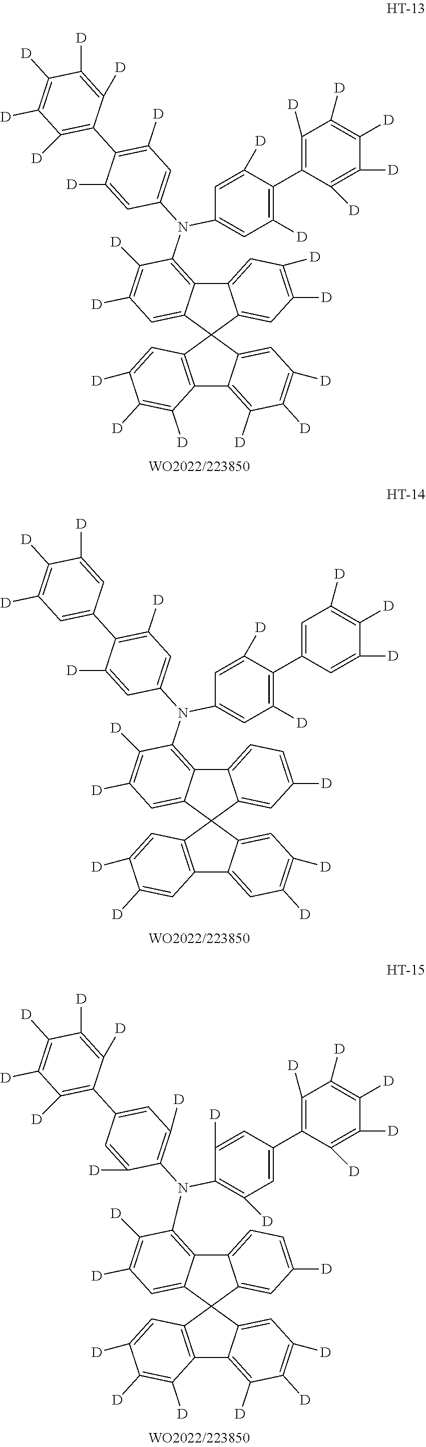

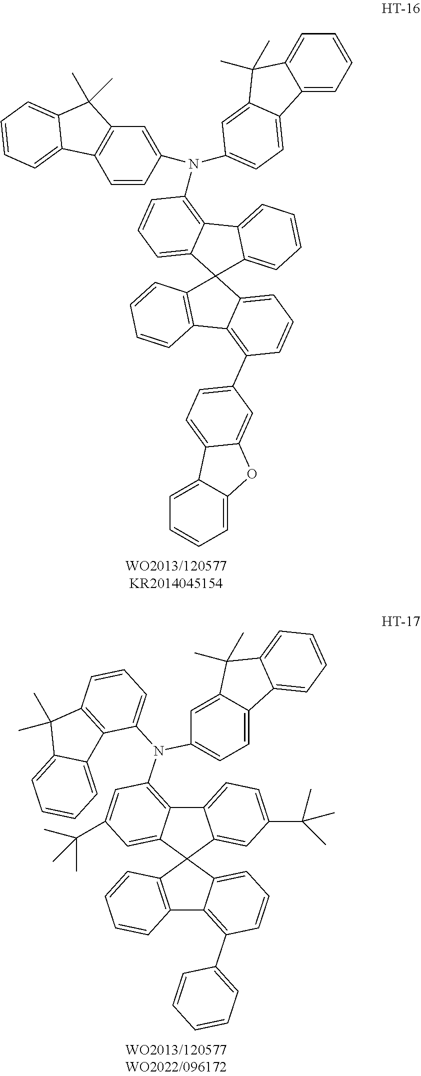 [SMILES: CC1(C)c2ccccc2-c2ccc(N(c3ccc4c(c3)C(C)(C)c3ccccc3-4)c3cccc4c3-c3ccccc3C43c4ccccc4-c4c(-c5ccc6c(c5)oc5ccccc56)cccc43)cc21 ; CC(C)(C)c1ccc2c(c1)C1(c3ccccc3-c3c(-c4ccccc4)cccc31)c1cc(C(C)(C)C)cc(N(c3ccc4c(c3)C(C)(C)c3ccccc3-4)c3cccc4c3-c3ccccc3C4(C)C)c1-2]

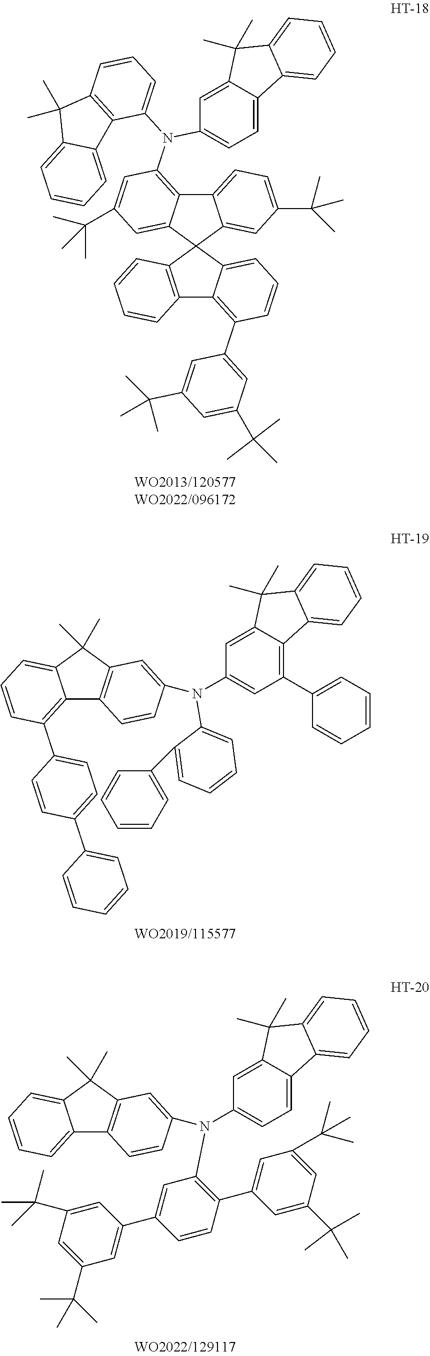

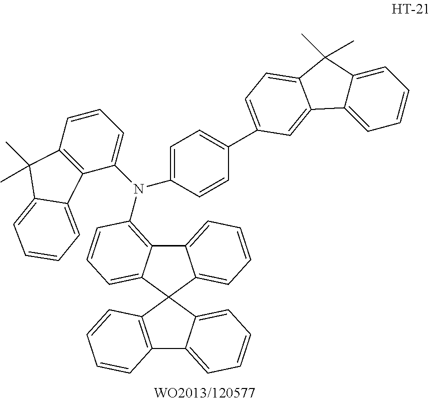 [SMILES: CC1(C)c2ccccc2-c2cc(-c3ccc(N(c4cccc5c4-c4ccccc4C5(C)C)c4cccc5c4-c4ccccc4C54c5ccccc5-c5ccccc54)cc3)ccc21]

Compounds HT-1 to HT-21 may generally be used in any hole transport layers of OLEDs. The term “hole transport layer” here means any layer of an OLED between anode and emitting layer. The term “OLED” is not especially restricted and applies to all OLEDs, especially OLED structures that were customary at the filing date of the present application.

The compounds HT-1 to HT-21 may be prepared by methods disclosed in the application texts listed in the above table under the respective compounds HT-1 to HT-21. The teaching relating to the use of the compounds and the processes for preparing the compounds in the abovementioned application texts are hereby explicitly incorporated into the present disclosure by reference. Compounds HT-1 to HT-21 have excellent properties when used in OLEDs, especially excellent lifetime and efficiency. This is the case especially when they are used in a hole transport layer of the OLED.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the outcoupling of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I) can be used in displays, as light sources in lighting applications and as light sources in medical and/or cosmetic applications.

The electronic device according to the application, comprising anode, cathode, an emitting layer and a layer containing a compound of the formula (I) between the anode and the emitting layer, preferably has one or more of the abovementioned preferred features relating to electronic devices containing a compound of the formula (I).

In particular, with regard to the electronic device according to the invention that contains a compound of the formula (I), it is preferable that this is an OLED. More particularly, with regard to the device, it is preferable that this has a blue-fluorescing emitting layer, and the compound of the formula (I) is in a hole-transporting layer which, on the anode side, adjoins a further layer which adjoins the emitting layer on the anode side. Alternatively, with regard to the device, it is preferable that this has a blue-fluorescing or green-phosphorescing emitting layer, and the compound of the formula (I) is in a hole-transporting layer which adjoins the emitting layer on the anode side.

### EXAMPLES

### A) Synthesis Examples

### Synthesis of Int-1a

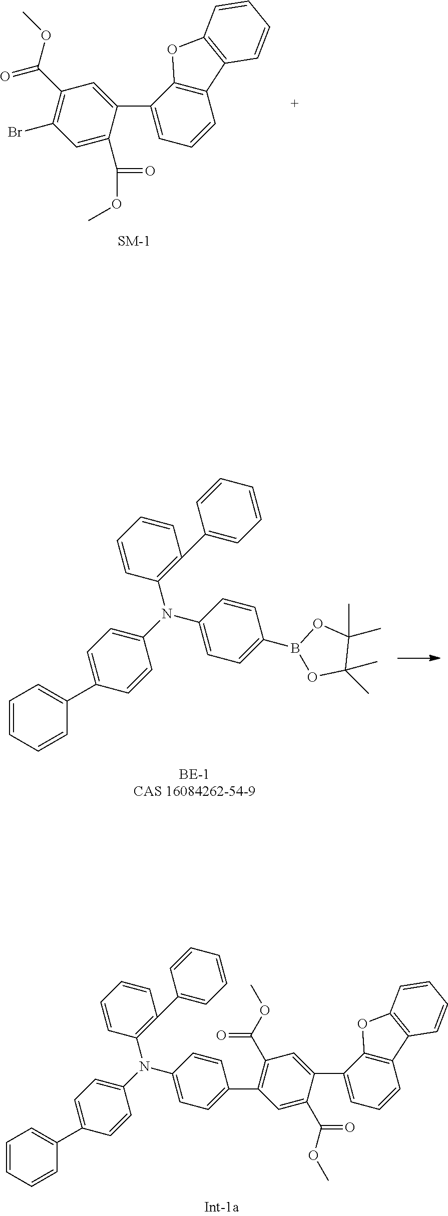 [SMILES: COC(=O)c1cc(-c2cccc3c2oc2ccccc23)c(C(=O)OC)cc1Br ; CC1(C)OB(c2ccc(N(c3ccc(-c4ccccc4)cc3)c3ccccc3-c3ccccc3)cc2)OC1(C)C ; COC(=O)c1cc(-c2cccc3c2oc2ccccc23)c(C(=O)OC)cc1-c1ccc(N(c2ccc(-c3ccccc3)cc2)c2ccccc2-c2ccccc2)cc1]

15.4 g (35 mmol) of SM-1, 19.3 g (37 mmol) of BE-1 and 16.0 g (70 mmol) of potassium phosphate monohydrate form an initial charge in 450 ml of THE/water (2:1). Subsequently, 883 mg (1.0 mmol) of XPhos palladacycle Gen. 3 is added and the mixture is stirred at 60° C. On completion of conversion, the reaction mixture is cooled down to room temperature and extracted twice with THF, dried over sodium sulfate, filtered and concentrated on a rotary evaporator. The residue is filtered through AlOx (heptane/toluene) and then concentrated. After crystallization from ethyl acetate and isopropanol, the product is isolated in solid form with an HPLC purity >96%.

Yield 20.9 g (27 mmol; 77%)

The following compounds can be prepared analogously:

| Compd. | SM | BE | Product |
|---|---|---|---|
| Int-1b | SM-1 | 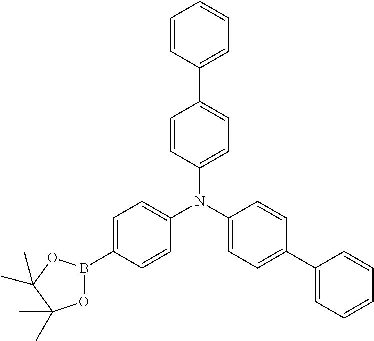 | 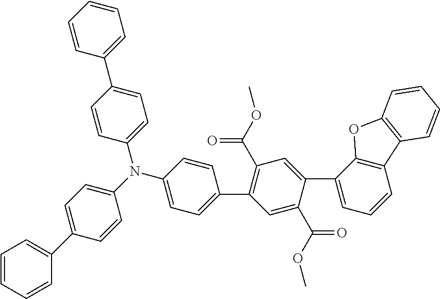 [SMILES: COC(=O)c1cc(-c2cccc3c2oc2ccccc23)c(C(=O)OC)cc1-c1ccc(N(c2ccc(-c3ccccc3)cc2)c2ccc(-c3ccccc3)cc2)cc1] |
| | | CAS 952431-30-0 | |
| Int-1c | SM-1 | 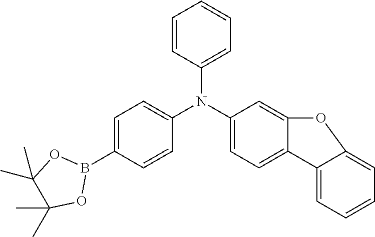 | 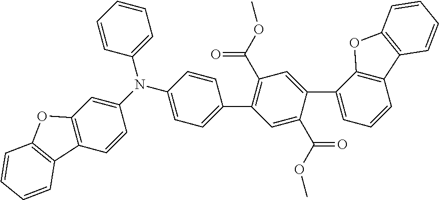 [SMILES: COC(=O)c1cc(-c2cccc3c2oc2ccccc23)c(C(=O)OC)cc1-c1ccc(N(c2ccccc2)c2ccc3c(c2)oc2ccccc23)cc1] |
| | | CAS 1613551-19-1 | |
| Int-1d | SM-1 | 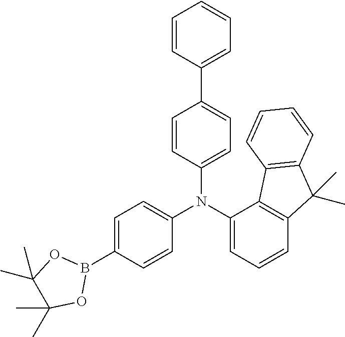 | 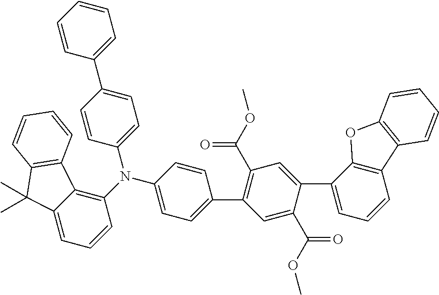 [SMILES: COC(=O)c1cc(-c2cccc3c2oc2ccccc23)c(C(=O)OC)cc1-c1ccc(N(c2ccc(-c3ccccc3)cc2)c2cccc3c2-c2ccccc2C3(C)C)cc1] |
| | | CAS 2102016-74-8 | |
| Int-1e | SM-1 | 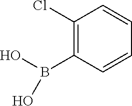 | 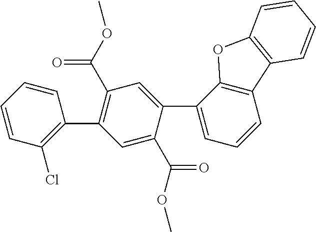 [SMILES: COC(=O)c1cc(-c2cccc3c2oc2ccccc23)c(C(=O)OC)cc1-c1ccccc1Cl] |
| | | CAS 3900-89-8 | |
| Int-1f | SM-1 | 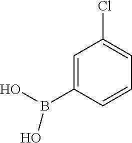 | 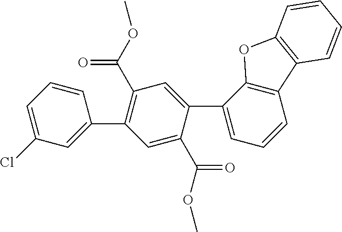 [SMILES: COC(=O)c1cc(-c2cccc3c2oc2ccccc23)c(C(=O)OC)cc1-c1cccc(Cl)c1] |
| | | CAS 63503-60-6 | |
| Int-1g | SM-1 | 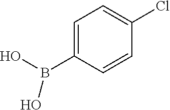 | 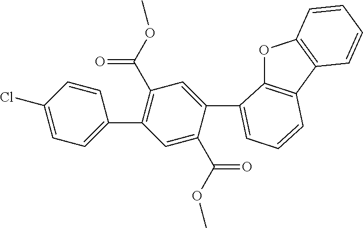 [SMILES: COC(=O)c1cc(-c2cccc3c2oc2ccccc23)c(C(=O)OC)cc1-c1ccc(Cl)cc1] |
| | | CAS 1679-18-1 | |
| Int-1h | SM-1 | 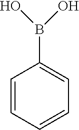 | 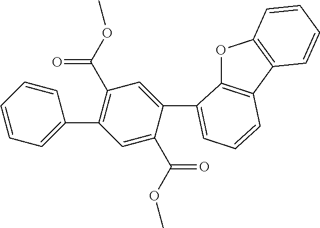 [SMILES: COC(=O)c1cc(-c2cccc3c2oc2ccccc23)c(C(=O)OC)cc1-c1ccccc1] |
| | | CAS 98-80-6 | |
| Int-1i | 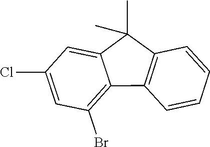 | 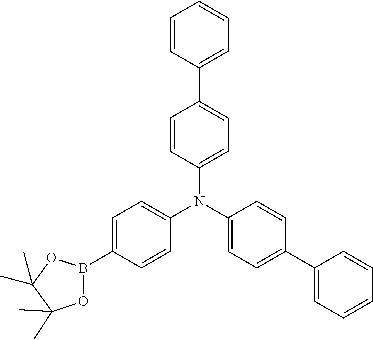 | 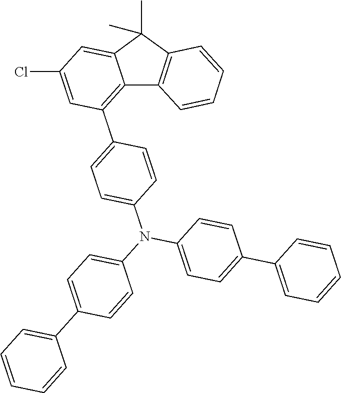 [SMILES: CC1(C)c2ccccc2-c2c(-c3ccc(N(c4ccc(-c5ccccc5)cc4)c4ccc(-c5ccccc5)cc4)cc3)cc(Cl)cc21] |
| | CAS 2043961- | CAS 952431-30-0 | |
| | 99-3 | | |
| Int-1j | 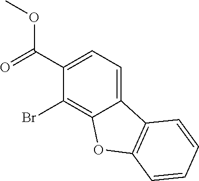 | 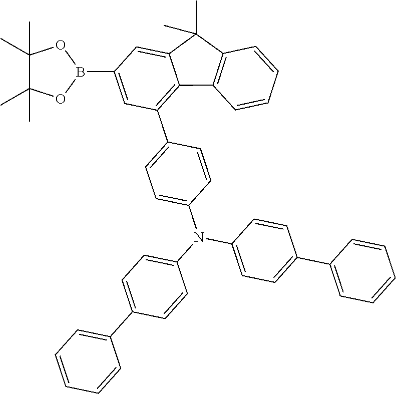 | 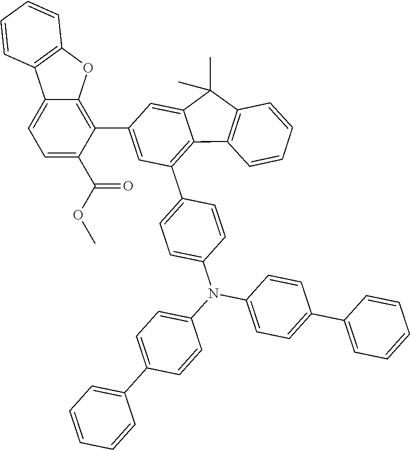 [SMILES: COC(=O)c1ccc2c(oc3ccccc32)c1-c1cc(-c2ccc(N(c3ccc(-c4ccccc4)cc3)c3ccc(-c4ccccc4)cc3)cc2)c2c(c1)C(C)(C)c1ccccc1-2] |
| | CN109824681 A | Int-1i-b* | |
| Int-1k | 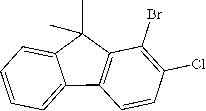 | 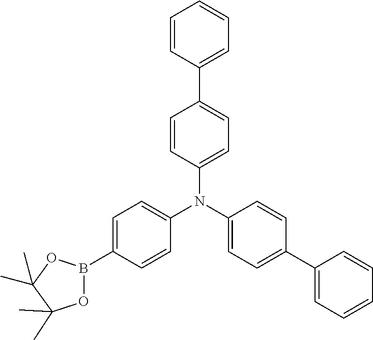 | 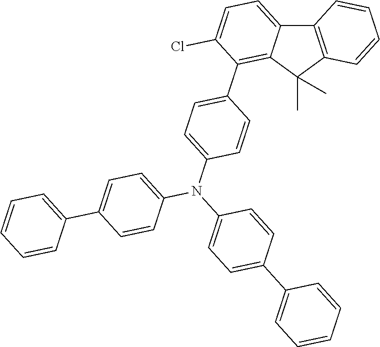 [SMILES: CC1(C)c2ccccc2-c2ccc(Cl)c(-c3ccc(N(c4ccc(-c5ccccc5)cc4)c4ccc(-c5ccccc5)cc4)cc3)c21] |
| | | CAS 952431-30-0 | |
| Int-1l | 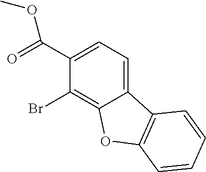 | 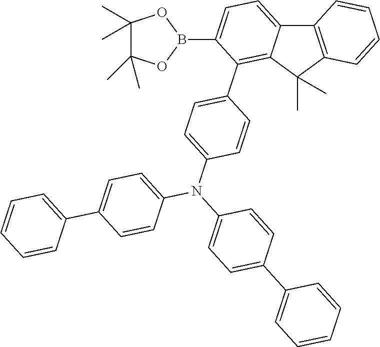 | 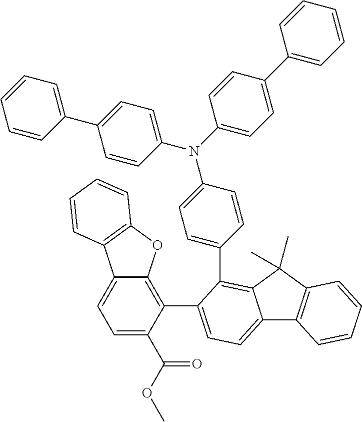 [SMILES: COC(=O)c1ccc2c(oc3ccccc32)c1-c1ccc2c(c1-c1ccc(N(c3ccc(-c4ccccc4)cc3)c3ccc(-c4ccccc4)cc3)cc1)C(C)(C)c1ccccc1-2] |
| | CN109824681 A | Int-1k-b* | |
| Int-1m | 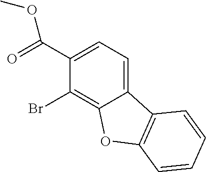 | 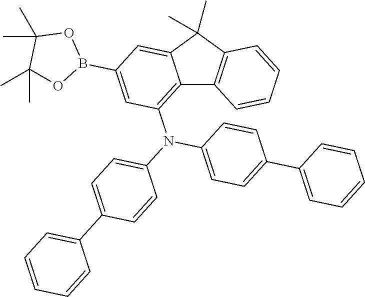 | 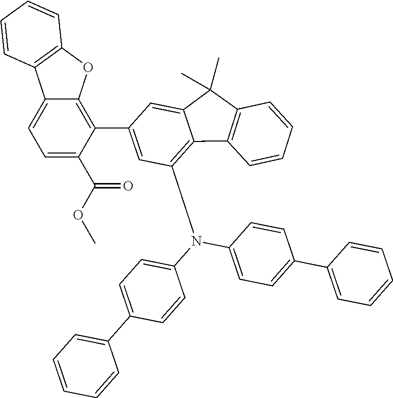 [SMILES: COC(=O)c1ccc2c(oc3ccccc32)c1-c1cc(N(c2ccc(-c3ccccc3)cc2)c2ccc(-c3ccccc3)cc2)c2c(c1)C(C)(C)c1ccccc1-2] |
| | CN109824681 A | Int-5-1-b* | |
| Int-1n | 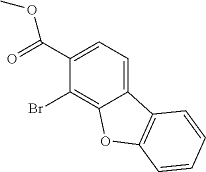 | 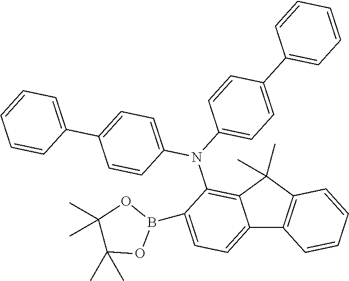 | 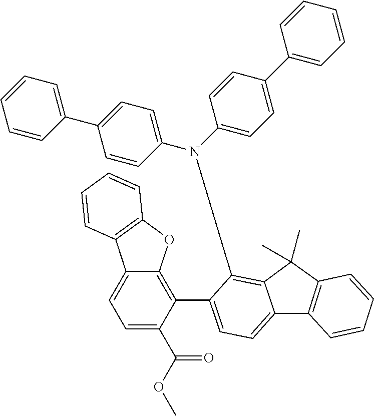 [SMILES: COC(=O)c1ccc2c(oc3ccccc32)c1-c1ccc2c(c1N(c1ccc(-c3ccccc3)cc1)c1ccc(-c3ccccc3)cc1)C(C)(C)c1ccccc1-2] |
| | CN109824681 A | Int-5-2-b* | |

Borylation in the reactions specified above proceeds analogously to that in the case of compound EG1 from WO2019/170572.

### Synthesis of Int-2a

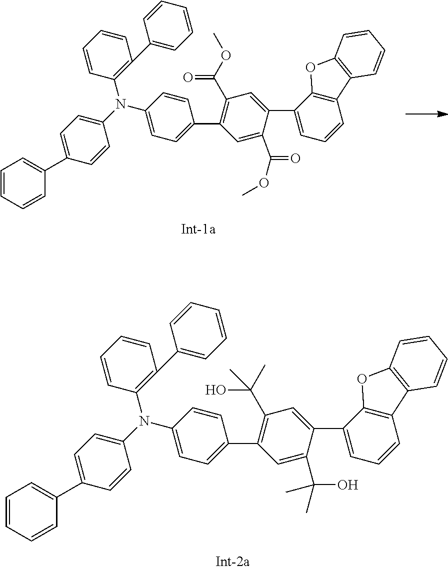 [SMILES: COC(=O)c1cc(-c2cccc3c2oc2ccccc23)c(C(=O)OC)cc1-c1ccc(N(c2ccc(-c3ccccc3)cc2)c2ccccc2-c2ccccc2)cc1 ; CC(C)(O)c1cc(-c2cccc3c2oc2ccccc23)c(C(C)(C)O)cc1-c1ccc(N(c2ccc(-c3ccccc3)cc2)c2ccccc2-c2ccccc2)cc1]

To an initial charge of 13.5 g (55 mmol) of cerium(III) chloride is added 20.2 g (27 mmol) of Int-1a dissolved in 300 ml of THF, and the mixture is stirred at room temperature for one hour. The reaction mixture is cooled down to 0° C. and, at that temperature, 51.4 ml (154 mmol) of methylmagnesium chloride (3M in THF) is added dropwise and the mixture is stirred at that temperature for 45 minutes. Subsequently, the reaction mixture is stirred at room temperature for a further two hours and, on completion of conversion, saturated aqueous ammonium chloride solution is added cautiously. The suspension is extended with 200 ml of water and extracted three times with 200 ml of THF. The combined organic phases are dried over sodium sulfate, filtered and concentrated, and the product is obtained in solid form.

Yield 20.4 g (27 mmol; 100%)

The following compounds can be prepared analogously:

| Compd. | Reactant | Product |
|---|---|---|
| Int-2b | Int-1b | 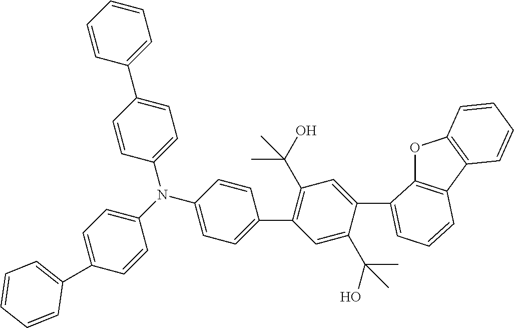 [SMILES: CC(C)(O)c1cc(-c2cccc3c2oc2ccccc23)c(C(C)(C)O)cc1-c1ccc(N(c2ccc(-c3ccccc3)cc2)c2ccc(-c3ccccc3)cc2)cc1] |
| Int-2c | Int-1c | 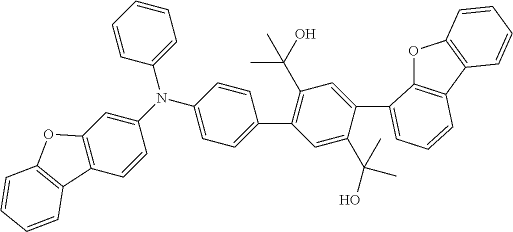 [SMILES: CC(C)(O)c1cc(-c2cccc3c2oc2ccccc23)c(C(C)(C)O)cc1-c1ccc(N(c2ccccc2)c2ccc3c(c2)oc2ccccc23)cc1] |
| Int-2d | Int-1d | 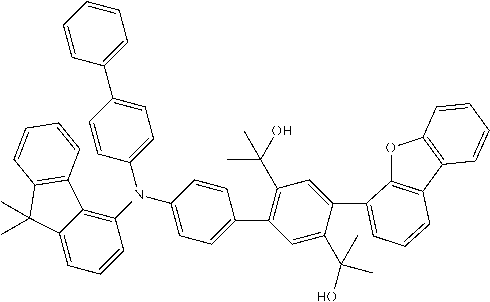 [SMILES: CC(C)(O)c1cc(-c2cccc3c2oc2ccccc23)c(C(C)(C)O)cc1-c1ccc(N(c2ccc(-c3ccccc3)cc2)c2cccc3c2-c2ccccc2C3(C)C)cc1] |
| Int-2e | Int-1e | 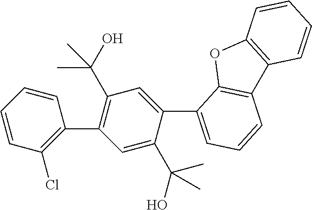 [SMILES: CC(C)(O)c1cc(-c2cccc3c2oc2ccccc23)c(C(C)(C)O)cc1-c1ccccc1Cl] |
| Int-2f | Int-1f | 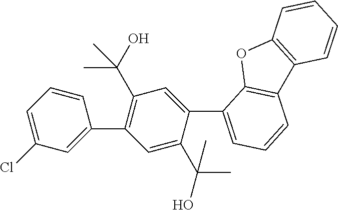 [SMILES: CC(C)(O)c1cc(-c2cccc3c2oc2ccccc23)c(C(C)(C)O)cc1-c1cccc(Cl)c1] |
| Int-2g | Int-1g | 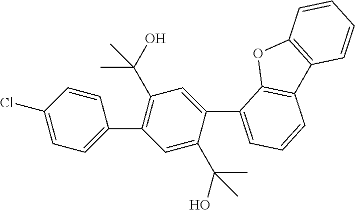 [SMILES: CC(C)(O)c1cc(-c2cccc3c2oc2ccccc23)c(C(C)(C)O)cc1-c1ccc(Cl)cc1] |
| Int-2h | Int-1h | 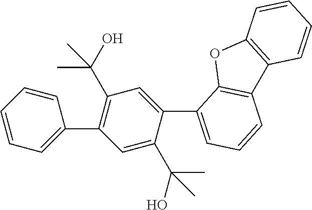 [SMILES: CC(C)(O)c1cc(-c2cccc3c2oc2ccccc23)c(C(C)(C)O)cc1-c1ccccc1] |
| Int-2i | Int-1j | 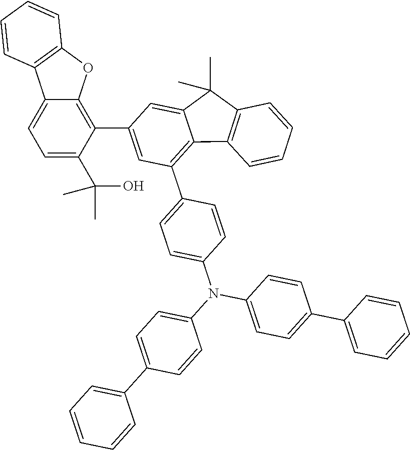 [SMILES: CC(C)(O)c1ccc2c(oc3ccccc32)c1-c1cc(-c2ccc(N(c3ccc(-c4ccccc4)cc3)c3ccc(-c4ccccc4)cc3)cc2)c2c(c1)C(C)(C)c1ccccc1-2] |
| Int-2j | Int-1l | 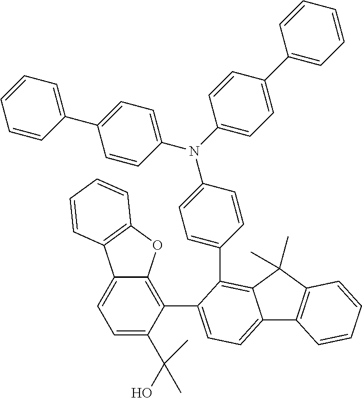 [SMILES: CC(C)(O)c1ccc2c(oc3ccccc32)c1-c1ccc2c(c1-c1ccc(N(c3ccc(-c4ccccc4)cc3)c3ccc(-c4ccccc4)cc3)cc1)C(C)(C)c1ccccc1-2] |
| Int-2k | Int-1m | 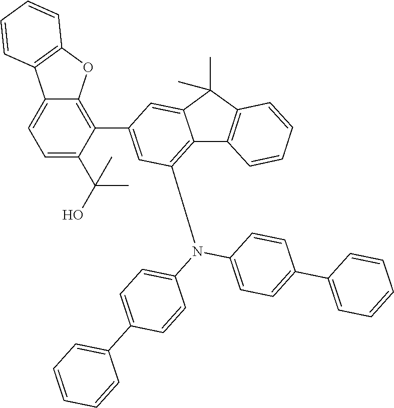 [SMILES: CC(C)(O)c1ccc2c(oc3ccccc32)c1-c1cc(N(c2ccc(-c3ccccc3)cc2)c2ccc(-c3ccccc3)cc2)c2c(c1)C(C)(C)c1ccccc1-2] |
| Int-2l | In-1n | 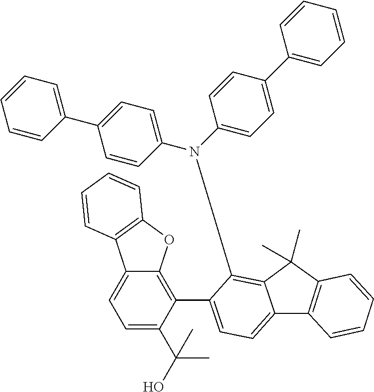 [SMILES: CC(C)(O)c1ccc2c(oc3ccccc32)c1-c1ccc2c(c1N(c1ccc(-c3ccccc3)cc1)c1ccc(-c3ccccc3)cc1)C(C)(C)c1ccccc1-2] |

### Synthesis of Compound 1a

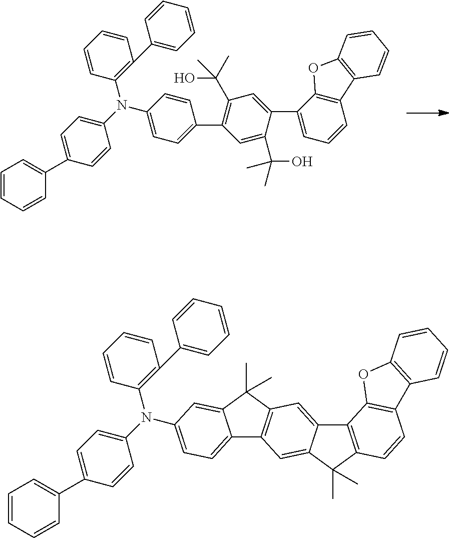 [SMILES: CC(C)(O)c1cc(-c2cccc3c2oc2ccccc23)c(C(C)(C)O)cc1-c1ccc(N(c2ccc(-c3ccccc3)cc2)c2ccccc2-c2ccccc2)cc1 ; CC1(C)c2cc(N(c3ccc(-c4ccccc4)cc3)c3ccccc3-c3ccccc3)ccc2-c2cc3c(cc21)-c1c(ccc2c1oc1ccccc12)C3(C)C]

To a solution of 20.2 g (27 mmol) of Int-2a in 500 ml of THE is added 3.7 g (38 mmol) of Amberlyst-15. The reaction mixture is stirred at 100° C. overnight until conversion is complete, and then filtered and concentrated on a rotary evaporator. The residue is twice subjected to hot extraction over AlOx with toluene and then crystallized repeatedly from toluene/heptane up to an HPLC purity >99.9%. After zone sublimation (10⁻⁶ bar, 340° C.), the product is obtained in solid form.

Yield: 10.1 g (14 mmol; 53%)

The following compounds can be prepared analogously:

| Compd. | Reactant | Product |
|---|---|---|
| 1b | Int-2b | 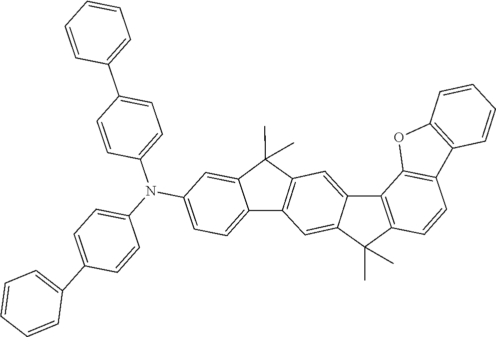 [SMILES: CC1(C)c2cc(N(c3ccc(-c4ccccc4)cc3)c3ccc(-c4ccccc4)cc3)ccc2-c2cc3c(cc21)-c1c(ccc2c1oc1ccccc12)C3(C)C] |
| 1c | Int-2c | 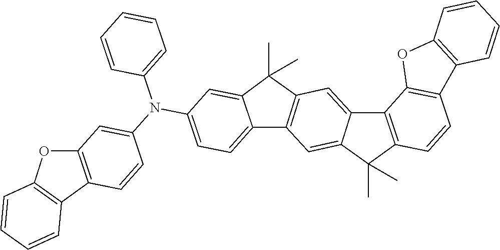 [SMILES: CC1(C)c2cc(N(c3ccccc3)c3ccc4c(c3)oc3ccccc34)ccc2-c2cc3c(cc21)-c1c(ccc2c1oc1ccccc12)C3(C)C] |
| 1d | Int-2d | 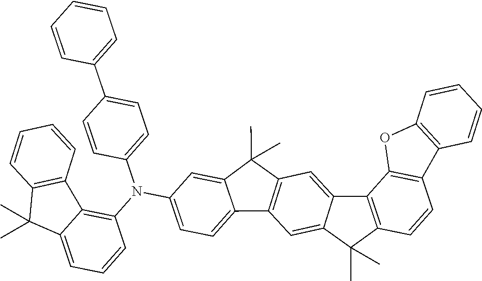 [SMILES: CC1(C)c2cc(N(c3ccc(-c4ccccc4)cc3)c3cccc4c3-c3ccccc3C4(C)C)ccc2-c2cc3c(cc21)-c1c(ccc2c1oc1ccccc12)C3(C)C] |
| 1e | Int-2i | 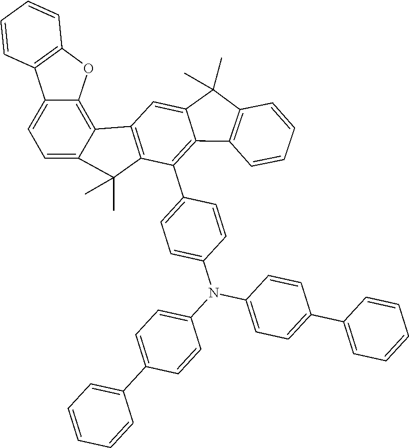 [SMILES: CC1(C)c2ccccc2-c2c1cc1c(c2-c2ccc(N(c3ccc(-c4ccccc4)cc3)c3ccc(-c4ccccc4)cc3)cc2)C(C)(C)c2ccc3c(oc4ccccc43)c2-1] |
| 1f | Int-2j | 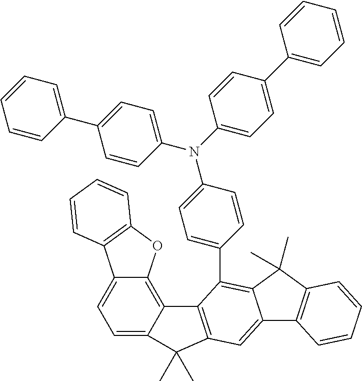 [SMILES: CC1(C)c2cc3c(c(-c4ccc(N(c5ccc(-c6ccccc6)cc5)c5ccc(-c6ccccc6)cc5)cc4)c2-c2c1ccc1c2oc2ccccc21)C(C)(C)c1ccccc1-3] |
| 1g | Int-2k | 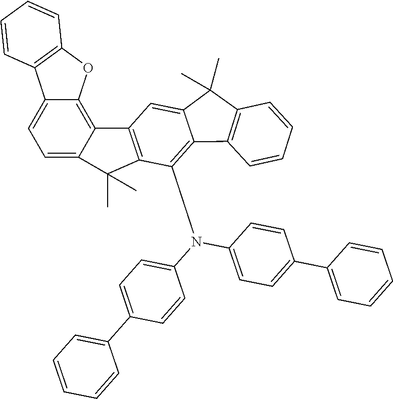 [SMILES: CC1(C)c2ccccc2-c2c1cc1c(c2N(c2ccc(-c3ccccc3)cc2)c2ccc(-c3ccccc3)cc2)C(C)(C)c2ccc3c(oc4ccccc43)c2-1] |
| 1h | Int-2l | 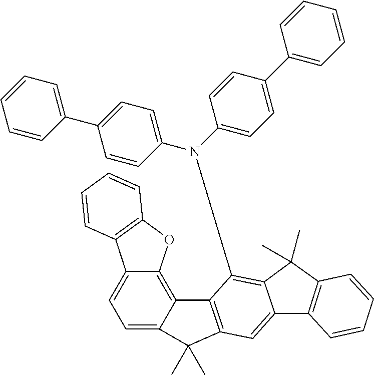 [SMILES: CC1(C)c2cc3c(c(N(c4ccc(-c5ccccc5)cc4)c4ccc(-c5ccccc5)cc4)c2-c2c1ccc1c2oc2ccccc21)C(C)(C)c1ccccc1-3] |
| Int-3a | Int-2e | 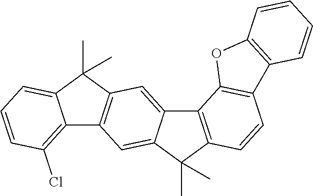 [SMILES: CC1(C)c2cc3c(cc2-c2c(Cl)cccc21)C(C)(C)c1ccc2c(oc4ccccc42)c1-3] |
| Int-3b-a and Int- 3b-b* | Int-2f | 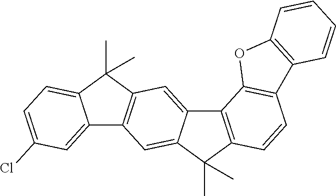 [SMILES: CC1(C)c2ccc(Cl)cc2-c2cc3c(cc21)-c1c(ccc2c1oc1ccccc12)C3(C)C] |
| | | Int-3b-a |
| | | 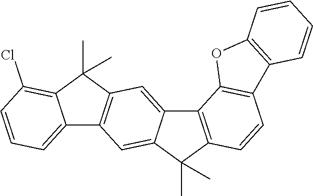 [SMILES: CC1(C)c2cc3c(cc2-c2c1ccc1c2oc2ccccc21)C(C)(C)c1c(Cl)cccc1-3] |
| | | Int-3b-b |
| Int-3c | Int-2g | 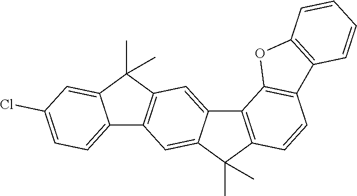 [SMILES: CC1(C)c2cc(Cl)ccc2-c2cc3c(cc21)-c1c(ccc2c1oc1ccccc12)C3(C)C] |
| Int-3d | Int-2h | 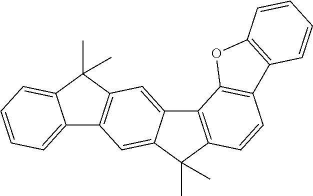 [SMILES: CC1(C)c2ccccc2-c2cc3c(cc21)-c1c(ccc2c1oc1ccccc12)C3(C)C] |

Int-3b-a and Int-3b-b are formed as an isomer mixture that can be separated by chromatography.

### Synthesis of 2a

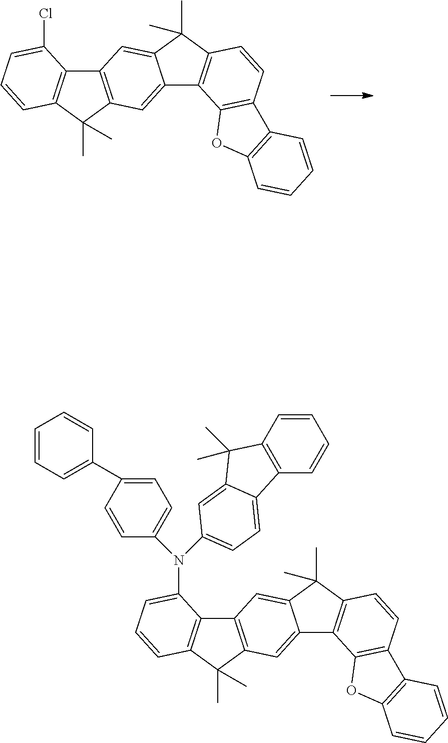 [SMILES: CC1(C)c2cc3c(cc2-c2c(Cl)cccc21)C(C)(C)c1ccc2c(oc4ccccc42)c1-3 ; CC1(C)c2ccccc2-c2ccc(N(c3ccc(-c4ccccc4)cc3)c3cccc4c3-c3cc5c(cc3C4(C)C)-c3c(ccc4c3oc3ccccc34)C5(C)C)cc21]

11.1 g (26 mmol) of Int-3a, 8.85 g (25 mmol) of 9,9-dimethylfluorenyl-2-(4-biphenyl)amine, 3.5 g (37 mmol) of sodium tert-butoxide and 616 mg (0.73 mmol) of XPhos Pd Gen3 are suspended in 400 ml of toluene and stirred at 100° C. for 16 hours. On completion of conversion, the reaction mixture is allowed to cool down to room temperature and filtered through aluminum oxide and washed with toluene. After the solvents have been removed, the crude product is dissolved in toluene/heptane 1:1 and filtered through silica gel. Further purification is effected by repeated crystallization from heptane/toluene up to an HPLC purity of >99.9%. Finally, the product, after two sublimations (310° C., 10⁻⁶ bar), is obtained in solid form.

Yield: 7.5 g (10 mmol; 39%)

The following compounds can be prepared analogously:

| Compd. | Amine | Reactant | Product |
|---|---|---|---|
| 2b | CAS 1879963- 55-9 | Int-3a | 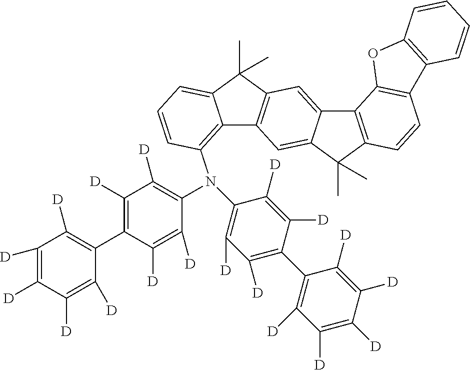 [SMILES: [2H]c1c([2H])c([2H])c(-c2c([2H])c([2H])c(N(c3cccc4c3-c3cc5c(cc3C4(C)C)-c3c(ccc4c3oc3ccccc34)C5(C)C)c3c([2H])c([2H])c(-c4c([2H])c([2H])c([2H])c([2H])c4[2H])c([2H])c3[2H])c([2H])c2[2H])c([2H])c1[2H]] |
| 2c | CAS 897671- 69-1 | Int-3b-a | 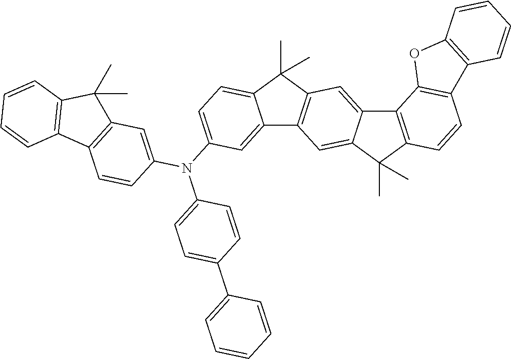 [SMILES: CC1(C)c2ccccc2-c2ccc(N(c3ccc(-c4ccccc4)cc3)c3ccc4c(c3)-c3cc5c(cc3C4(C)C)-c3c(ccc4c3oc3ccccc34)C5(C)C)cc21] |
| 2d | CAS 102113- 98-4 | Int-3c | 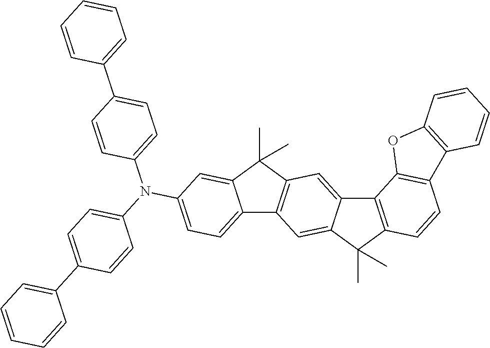 [SMILES: CC1(C)c2cc(N(c3ccc(-c4ccccc4)cc3)c3ccc(-c4ccccc4)cc3)ccc2-c2cc3c(cc21)-c1c(ccc2c1oc1ccccc12)C3(C)C] |
| 2e | CAS 102113- 98-4 | Int-3b-b | 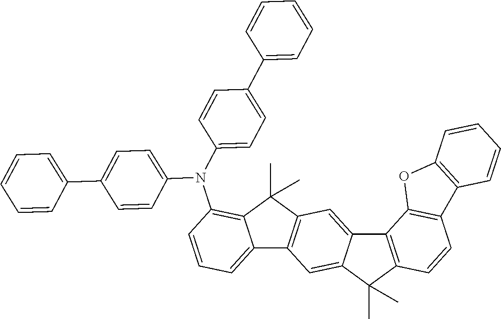 [SMILES: CC1(C)c2cc3c(cc2-c2c1ccc1c2oc2ccccc21)C(C)(C)c1c-3cccc1N(c1ccc(-c2ccccc2)cc1)c1ccc(-c2ccccc2)cc1] |
| 2f | CAS 102113- 98-4 | CAS 2412533- 15-2 | 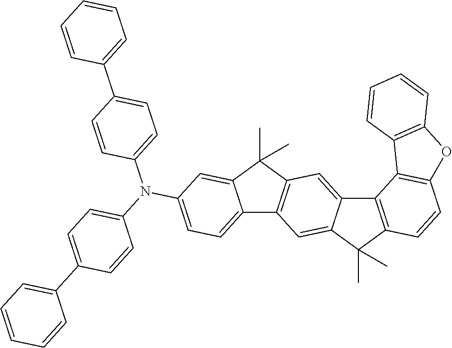 [SMILES: CC1(C)c2cc(N(c3ccc(-c4ccccc4)cc3)c3ccc(-c4ccccc4)cc3)ccc2-c2cc3c(cc21)-c1c(ccc2oc4ccccc4c12)C3(C)C] |
| 2g | CAS 955959- 89-4 | CAS 2412533- 15-2 | 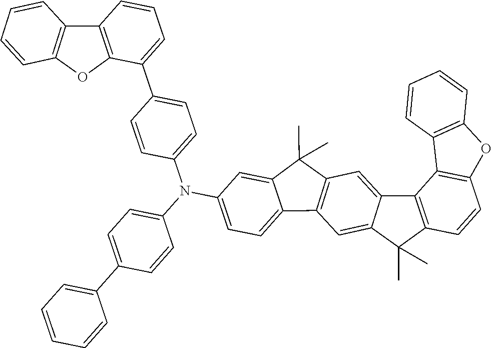 [SMILES: CC1(C)c2cc(N(c3ccc(-c4ccccc4)cc3)c3ccc(-c4cccc5c4oc4ccccc45)cc3)ccc2-c2cc3c(cc21)-c1c(ccc2oc4ccccc4c12)C3(C)C] |
| Int-5-1 | CAS 102113- 98-4 | 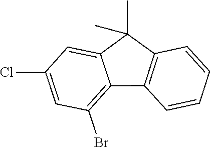 [SMILES: CC1(C)c2ccccc2-c2c(Br)cc(Cl)cc21] | 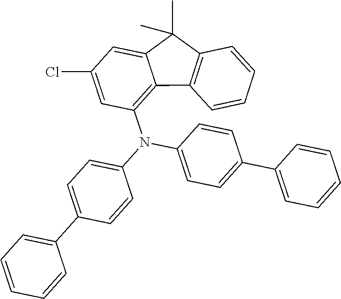 [SMILES: CC1(C)c2ccccc2-c2c(N(c3ccc(-c4ccccc4)cc3)c3ccc(-c4ccccc4)cc3)cc(Cl)cc21] |
| | | CAS 2043961- | |
| | | 99-3 | |
| Int-5-2 | CAS 102113- 98-4 | 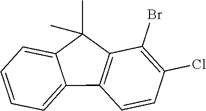 [SMILES: CC1(C)c2ccccc2-c2ccc(Cl)c(Br)c21] | 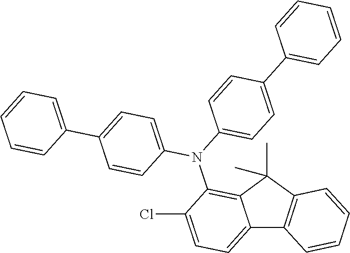 [SMILES: CC1(C)c2ccccc2-c2ccc(Cl)c(N(c3ccc(-c4ccccc4)cc3)c3ccc(-c4ccccc4)cc3)c21] |

### Synthesis of Int-4

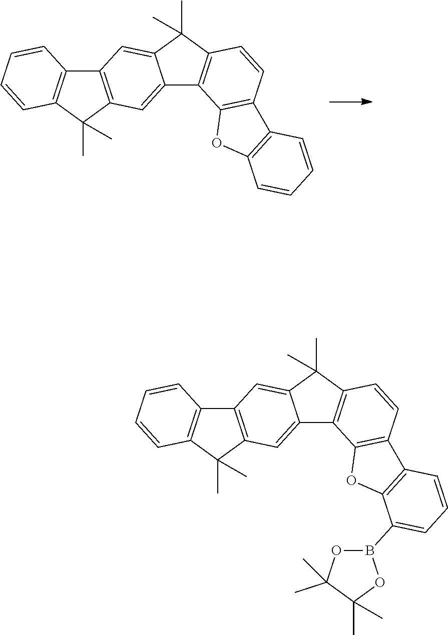 [SMILES: CC1(C)c2ccccc2-c2cc3c(cc21)-c1c(ccc2c1oc1ccccc12)C3(C)C ; CC1(C)c2ccccc2-c2cc3c(cc21)-c1c(ccc2c1oc1c(B4OC(C)(C)C(C)(C)O4)cccc12)C3(C)C]

16.2 g (40 mmol) of Int-3d is suspended in 600 ml of THE and cooled down to −45° C., 31 ml (43 mmol) of sec-BuLi (1.4M in cyclohexane) is slowly added dropwise and, on completion of addition, the mixture is stirred at −20° C. for one hour. Subsequently, the reaction mixture is cooled down to −35° C. and 17.8 ml (80 mmol) of 2-isopropoxy-4,4,5,5-tetramethyl-[1,3,2]dioxaborolane is added dropwise and the mixture is stirred at −20° C. for 30 minutes. The reaction mixture is allowed to come to room temperature overnight. 50 ml of saturated aqueous ammonium chloride solution is added gradually to the reaction mixture, and the organic phase is extended with ethyl acetate. The organic phase is washed twice with 200 ml of water, filtered and dried over sodium sulfate. The solvents are removed on a rotary evaporator, and the product is obtained in solid form.

Yield: 17.5 g (33.4 mmol; 84%)

### Synthesis of 3a

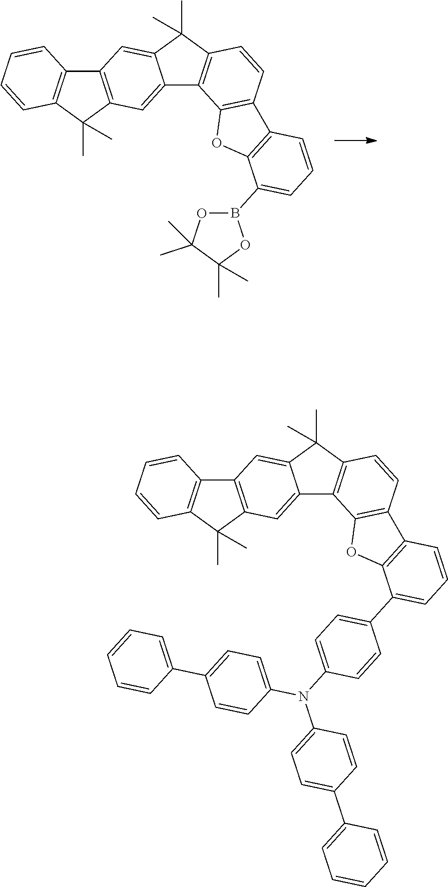 [SMILES: CC1(C)c2ccccc2-c2cc3c(cc21)-c1c(ccc2c1oc1c(B4OC(C)(C)C(C)(C)O4)cccc12)C3(C)C ; CC1(C)c2ccccc2-c2cc3c(cc21)-c1c(ccc2c1oc1c(-c4ccc(N(c5ccc(-c6ccccc6)cc5)c5ccc(-c6ccccc6)cc5)cc4)cccc12)C3(C)C]

12.1 g (23 mmol) of Int-4, 11.0 g (23 mmol) of bis(biphenyl-4-yl)(4-bromophenyl)amine (CAS 499128-71-1) and 8.8 g (23 mmol) of potassium phosphate monohydrate are suspended in 250 ml of THE/water (4:1). After addition of 483 mg (0.57 mmol) of XPhos Palladacycle Gen3, the reaction mixture is stirred at 60° C. overnight until conversion is complete. The reaction mixture is left to cool down to room temperature, and the precipitated solids are filtered off and washed with water, isopropanol and finally with heptane. The residue is four times subjected to hot extraction over AlOx with toluene and crystallized therefrom up to an HPLC purity >99.9%. After zone sublimation (10⁻⁶ bar, 360° C.), the product is obtained in solid form.

The following compounds can be prepared analogously:

| Compd. | Amine | Reactant | Product |
|---|---|---|---|
| 3b | CAS 1246562-40-2 | Int-4 | 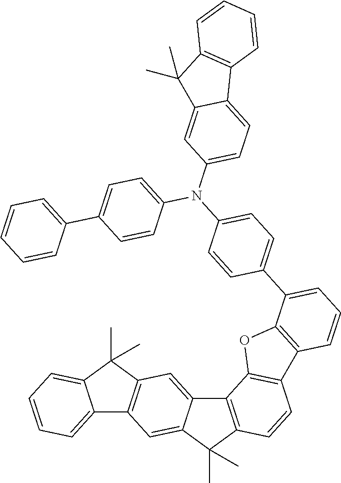 [SMILES: CC1(C)c2ccccc2-c2ccc(N(c3ccc(-c4ccccc4)cc3)c3ccc(-c4cccc5c4oc4c6c(ccc45)C(C)(C)c4cc5c(cc4-6)C(C)(C)c4ccccc4-5)cc3)cc21] |
| 3c | CAS 1443130-62-8 | Int-4 | 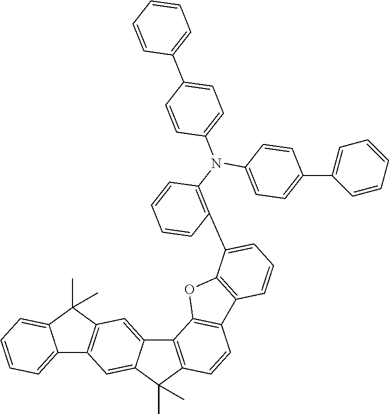 [SMILES: CC1(C)c2ccccc2-c2cc3c(cc21)-c1c(ccc2c1oc1c(-c4ccccc4N(c4ccc(-c5ccccc5)cc4)c4ccc(-c5ccccc5)cc4)cccc12)C3(C)C] |
| 3d | CAS 1160294-67-6 | Int-4 | 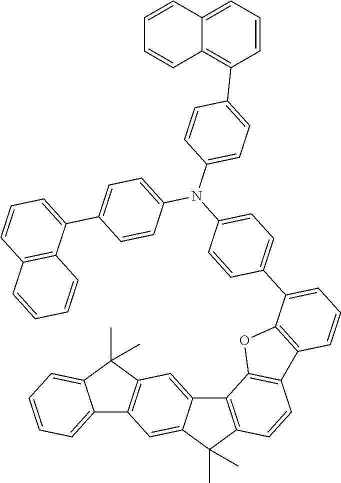 [SMILES: CC1(C)c2ccccc2-c2cc3c(cc21)-c1c(ccc2c1oc1c(-c4ccc(N(c5ccc(-c6cccc7ccccc67)cc5)c5ccc(-c6cccc7ccccc67)cc5)cc4)cccc12)C3(C)C] |

### B) Device Examples

### 1) General Production Process for the OLEDs and Characterization of the OLEDs

Glass plates which have been coated with structured ITO (indium tin oxide) in a thickness of 50 nm form the substrates to which the OLEDs are applied.

The OLEDs basically have the following layer structure: substrate/hole injection layer (HIL)/hole transport layer (HTL1)/optional second hole transport layer (HTL2)/electron blocker layer (EBL)/emission layer (EML)/optional hole blocker layer (HBL)/electron transport layer (ETL1)/optional second electron transport layer (ETL2)/electron injection layer (EIL) and finally a cathode. The cathode is formed by an aluminum layer of thickness 100 nm. The exact structure of the OLEDs can be found in the tables which follow. The materials required for production of the OLEDs are shown in a table below.

All materials are applied by thermal vapor deposition in a vacuum chamber. In this case, the emission layer consists of at least one matrix material (host material) and an emitting dopant (emitter) which is added to the matrix material(s) in a particular proportion by volume by co-evaporation. Details given in such a form as H:SEB (95%:5%) mean here that the material H is present in the layer in a proportion by volume of 95% and SEB in a proportion of 5%. In an analogous manner, the electron transport layer and the hole injection layer also consist of a mixture of two materials. The structures of the materials that are used in the OLEDs are shown in the table below. The ETM-2 used as electron transport material in OLED example 11 is a spirobifluorenyl-triazine derivative. The EBM-2 used as material of the electron blocker layer in OLED example 11 is a triarylamino-substituted spirobifluorene.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @10 mA/cm² refers to the external quantum efficiency which is attained at 10 mA/cm². The lifetime LT is defined as the time after which the luminance drops from the starting luminance to a certain proportion in the course of operation with constant current density. An LT90 figure means here that the lifetime reported corresponds to the time after which the luminance has dropped to 90% of its starting value. The figure @60 mA/cm² means here that the lifetime in question is measured at 60 mA/cm².

### 2) Use in the Electron Blocker Layer of a Blue-Fluorescing OLED:

OLEDs are produced with the following structure:

TABLE 1A
OLED structure

| | HIL | HTL1 | HTL2 | EBL | EML | ETL1 | EIL |
|---|---|---|---|---|---|---|---|
| | Thick- | Thick- | Thick- | Thick- | Thick- | Thick- | Thick- |
| Ex. | ness/nm | ness/nm | ness/nm | ness/nm | ness/nm | ness/nm | ness/nm |
| 1 | HTM: | HTM | HTM-1: | HTM-1 | H:SEB | ETM:LiQ | LiQ |
| | p-dopant | 160 nm | p-dopant | 10 nm | (95%:5%) | (50:50%) | 1 nm |
| | (5%) | | (5%) | | 20 nm | 30 nm | |
| | 20 nm | | 20 nm | | | | |
| 2 | HTM: | HTM | HTM-2: | HTM-2 | H:SEB | ETM:LiQ | LiQ |
| | p-dopant | 160 nm | p-dopant | 10 nm | (95%:5%) | (50:50%) | 1 nm |
| | (5%) | | (5%) | | 20 nm | 30 nm | |
| | 20 nm | | 20 nm | | | | |
| 3 | HTM: | HTM | HTM-3: | HTM-3 | H:SEB | ETM:LiQ | LiQ |
| | p-dopant | 160 nm | p-dopant | 10 nm | (95%:5%) | (50:50%) | 1 nm |
| | (5%) | | (5%) | | 20 nm | 30 nm | |
| | 20 nm | | 20 nm | | | | |

OLEDs 1-3 show that the compounds of the invention are of excellent suitability as materials in OLEDs. The OLEDs show very good properties as hole transport materials in this construction and achieve very high external quantum efficiencies and low operating voltage and very good lifetime:

TABLE 1B
OLED data

| Ex. | V | EQE | LT90 |
|---|---|---|---|
| 1 | 3.8 | 7.6 | 150 |
| 2 | 3.8 | 7.8 | 130 |
| 3 | 3.6 | 6.9 | 170 |

### 3) Use in the Hole Transport and Electron Blocker Layer of a Green-Phosphorescing OLED:

OLEDs are produced with the following structure:

TABLE 2A
OLED structure

| | HIL | HTL1 | EBL | EML | ETL1 | ETL2 | EIL |
|---|---|---|---|---|---|---|---|
| | Thick- | Thick- | Thick- | Thick- | Thick- | Thick- | Thick- |
| Ex. | ness/nm | ness/nm | ness/nm | ness/nm | ness/nm | ness/nm | ness/nm |
| 4 | HTM: | HTM | HTM-1 | TMM-1 (59%): | ETM | ETM:LiQ | LiQ |
| | p-dopant | 220 nm | 10 nm | TMM-2(29%): | 10 nm | (50:50%) | 1 nm |
| | (5%) | | | TEG(12%) | | 30 nm | |
| | 20 nm | | | 30 nm | | | |
| 5 | HTM: | HTM | HTM-2 | TMM-1 (59%): | ETM | ETM:LiQ | LiQ |
| | p-dopant | 220 nm | 10 nm | TMM-2(29%): | 10 nm | (50:50%) | 1 nm |
| | (5%) | | | TEG(12%) | | 30 nm | |
| | 20 nm | | | 30 nm | | | |
| 6 | HTM: | HTM | HTM-3 | TMM-1 (59%): | ETM | ETM:LiQ | LiQ |
| | p-dopant | 220 nm | 10 nm | TMM-2(29%): | 10 nm | (50:50%) | 1 nm |
| | (5%) | | | TEG(12%) | | 30 nm | |
| | 20 nm | | | 30 nm | | | |
| 7 | HTM: | HTM | HTM-4 | TMM-1 (59%): | ETM | ETM:LiQ | LiQ |
| | p-dopant | 220 nm | 10 nm | TMM-2(29%): | 10 nm | (50:50%) | 1 nm |
| | (5%) | | | TEG(12%) | | 30 nm | |
| | 20 nm | | | 30 nm | | | |

OLEDs 4-7 show that the compounds of the invention are of excellent suitability as materials of the electron blocker layer for green-phosphorescing OLEDs. The OLEDs show very good properties as hole transport or electron blocker materials and achieve low operating voltages with good external quantum efficiencies and excellent lifetime:

TABLE 2B
OLED data

| Ex. | V | EQE | LT90 |
|---|---|---|---|
| 4 | 3.8 | 15.4 | 110 |
| 5 | 3.8 | 15.8 | 130 |
| 6 | 3.9 | 16.7 | 80 |
| 7 | 4.0 | 15.0 | 150 |

### 3) Use in the Hole Transport Layer of Blue-Fluorescing OLEDs

OLEDs are produced with the following structure:

TABLE 3A
OLED structure

| | HIL | HTL1 | EBL | EML | ETL1 | EIL |
|---|---|---|---|---|---|---|
| | Thick- | Thick- | Thick- | Thick- | Thick- | Thick- |
| Ex. | ness/nm | ness/nm | ness/nm | ness/nm | ness/nm | ness/nm |
| 8 | HTM-1: | HTM-1 | EBM | H:SEB | ETM:LiQ | LiQ |
| | p-dopant | 180 nm | 10 nm | (95%:5%) | (50:50%) | 1 nm |
| | (5%) | | | 20 nm | 30 nm | |
| | 20 nm | | | | | |
| 9 | HTM-2: | HTM-2 | EBM | H:SEB | ETM:LiQ | LiQ |
| | p-dopant | 180 nm | 10 nm | (95%:5%) | (50:50%) | 1 nm |
| | (5%) | | | 20 nm | 30 nm | |
| | 20 nm | | | | | |
| 10 | HTM-4: | HTM-4 | EBM | H:SEB | ETM:LiQ | LiQ |
| | p-dopant | 180 nm | 10 nm | (95%:5%) | (50:50%) | 1 nm |
| | (5%) | | | 20 nm | 30 nm | |
| | 20 nm | | | | | |

OLEDs 8-10 show that the compounds of the invention are of excellent suitability as hole transport material for blue-fluorescing OLEDs. The OLEDs show very good properties as hole transport material and achieve low operating voltages with good external quantum efficiencies and excellent lifetime:

TABLE 3B
OLED data

| Ex. | V | EQE | LT90 |
|---|---|---|---|
| 8 | 4.9 | 8.5 | 90 |
| 9 | 4.6 | 8.8 | 110 |
| 10 | 4.1 | 7.4 | 140 |

In addition, the following blue-fluorescing OLED is produced:

TABLE 4A
OLED structure

| | HIL | HTL1 | EBL | EML | ETL1 | ETL2 | EIL |
|---|---|---|---|---|---|---|---|
| | Thick- | Thick- | Thick- | Thick- | Thick- | Thick- | Thick- |
| Ex. | ness/nm | ness/nm | ness/nm | ness/nm | ness/nm | ness/nm | ness/nm |
| 11 | HTM-5: | HTM-5 | EBM-2 | H-2:SEB-2 | HBM | ETM-2:LiQ | LiQ |
| | p-dopant | 185 nm | 5 nm | (95%:5%) | 5 nm | (50:50%) | 1 nm |
| | (5%) | | | 20 nm | | 30 nm | |
| | 10 nm | | | | | | |

OLED 11 shows that the compounds of the invention are of excellent suitability as hole transport material for blue-fluorescing OLEDs. The OLED shows very good properties as hole transport material and achieves a low operating voltage with good external quantum efficiency and excellent lifetime:

TABLE 4B
OLED data

| Ex. | V | EQE | LT90 |
|---|---|---|---|
| 11 | 4.1 | 7.8 | 179 |

TABLE 5
Materials used

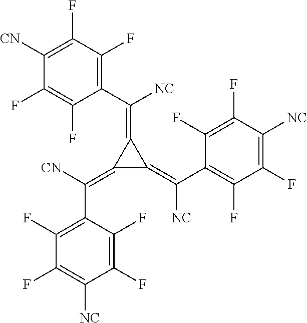

p-Dopant

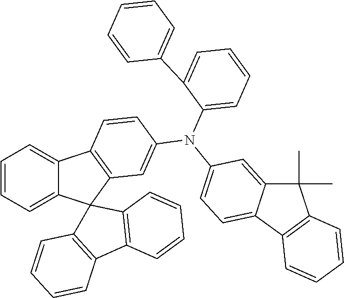 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTM

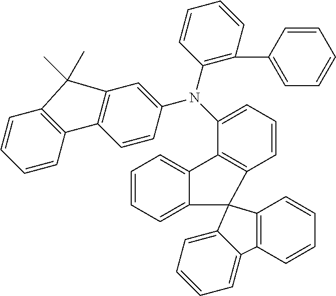 [SMILES: CC1(C)c2ccccc2-c2ccc(N(c3ccccc3-c3ccccc3)c3cccc4c3-c3ccccc3C43c4ccccc4-c4ccccc43)cc21]

EBM

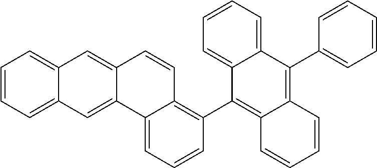 [SMILES: c1ccc(-c2c3ccccc3c(-c3cccc4c3ccc3cc5ccccc5cc34)c3ccccc23)cc1]

H

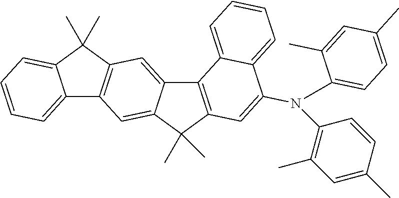 [SMILES: Cc1ccc(N(c2ccc(C)cc2C)c2cc3c(c4ccccc24)-c2cc4c(cc2C3(C)C)-c2ccccc2C4(C)C)c(C)c1]

SEB

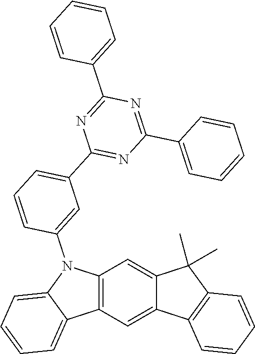 [SMILES: CC1(C)c2ccccc2-c2cc3c4ccccc4n(-c4cccc(-c5nc(-c6ccccc6)nc(-c6ccccc6)n5)c4)c3cc21]

TMM-1

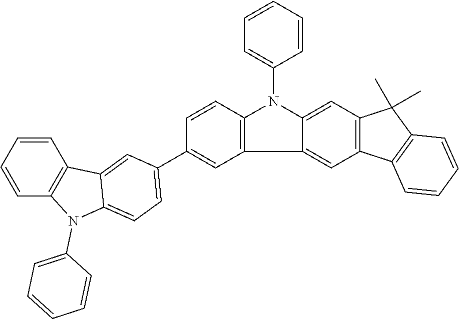 [SMILES: CC1(C)c2ccccc2-c2cc3c4cc(-c5ccc6c(c5)c5ccccc5n6-c5ccccc5)ccc4n(-c4ccccc4)c3cc21]

TMM-2

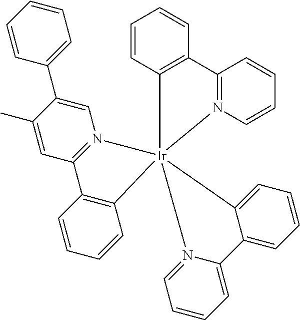

TEG

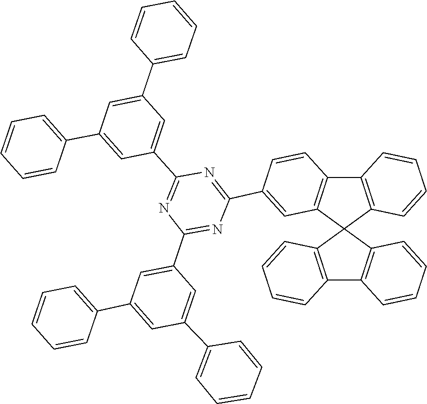 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ETM

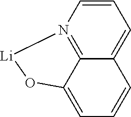

LiQ

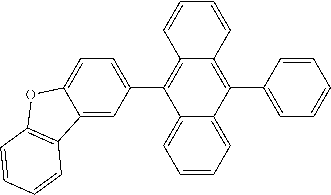 [SMILES: c1ccc(-c2c3ccccc3c(-c3ccc4oc5ccccc5c4c3)c3ccccc23)cc1]

H-2

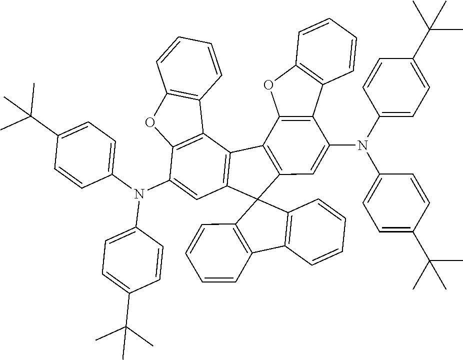 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C32c3ccccc3-c3ccccc32)cc1]

SEB-2

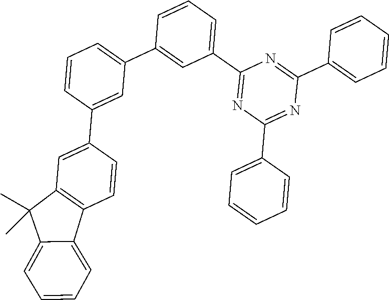 [SMILES: CC1(C)c2ccccc2-c2ccc(-c3cccc(-c4cccc(-c5nc(-c6ccccc6)nc(-c6ccccc6)n5)c4)c3)cc21]

HBM

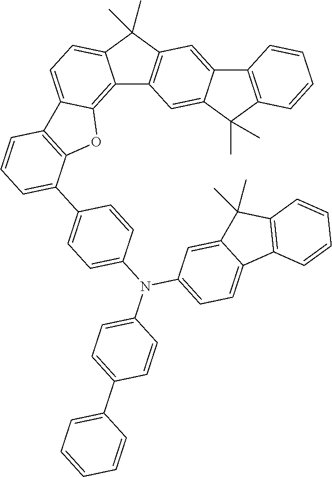 [SMILES: CC1(C)c2ccccc2-c2ccc(N(c3ccc(-c4ccccc4)cc3)c3ccc(-c4cccc5c4oc4c6c(ccc45)C(C)(C)c4cc5c(cc4-6)C(C)(C)c4ccccc4-5)cc3)cc21]

HTM-1

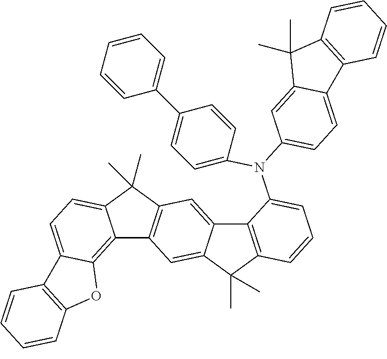

HTM-2

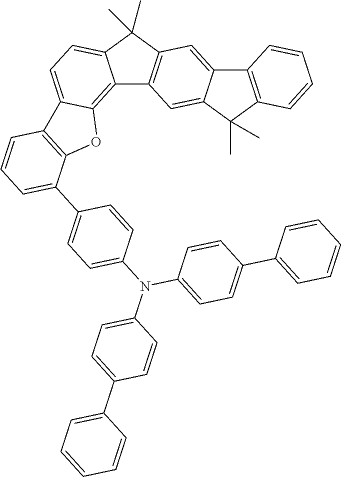 [SMILES: CC1(C)c2ccccc2-c2cc3c(cc21)-c1c(ccc2c1oc1c(-c4ccc(N(c5ccc(-c6ccccc6)cc5)c5ccc(-c6ccccc6)cc5)cc4)cccc12)C3(C)C]

HTM-3

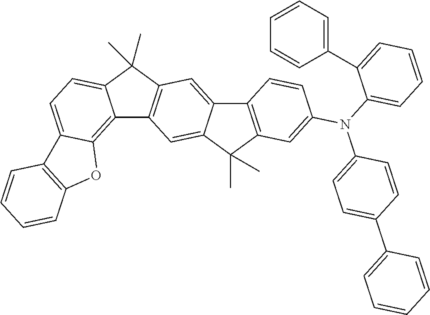

HTM-4

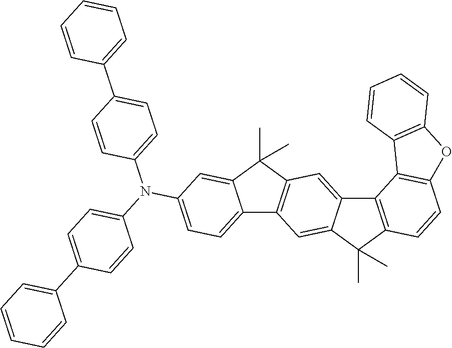 [SMILES: CC1(C)c2cc(N(c3ccc(-c4ccccc4)cc3)c3ccc(-c4ccccc4)cc3)ccc2-c2cc3c(cc21)-c1c(ccc2oc4ccccc4c12)C3(C)C]

HTM-5

1.-16. (canceled)

17. An electronic device comprising anode, cathode, an emitting layer, and a layer comprising a compound of the formula (I) disposed between the anode and the emitting layer, 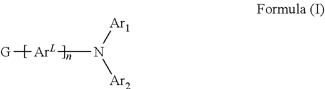
where the variables that occur are as follows:
G is a group of formula (G) which is bonded to the remainder of the formula (I) via one of the unoccupied positions on the benzene rings, where the other unoccupied positions on the benzene rings are each substituted by an R¹ radical; 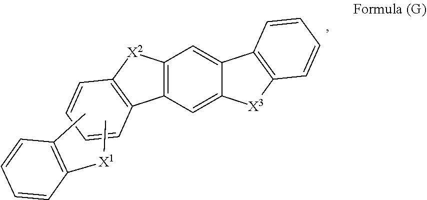
X¹ is O or S;
X² is C(R²)₂ or Si(R²)₂;
X³ is C(R²)₂ or Si(R²)₂;
Ar^{L} is an aromatic ring system which has 6 to 40 aromatic ring atoms and is substituted by R³ radicals, or a heteroaromatic ring system which has 5 to 40 aromatic ring atoms and is substituted by R³ radicals;
Ar¹, Ar² is the same or different and is an aromatic ring system which has 6 to 40 aromatic ring atoms and is substituted by R⁴ radicals, or a heteroaromatic ring system which has 5 to 25 aromatic ring atoms and is substituted by R⁴ radicals, where Ar¹ and Ar² may optionally be joined together via a bond or an E group;
E is C(R⁴)₂, —C(R⁴)₂—C(R⁴)₂—, —C(R⁴)═C(R⁴)—, C═O, Si(R⁴)₂, NR⁴, O, S, S═O, or SO₂;
R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R¹, S(═O)₂R³, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where the alkyl, alkoxy, alkenyl and alkynyl groups mentioned and the aromatic ring systems and heteroaromatic ring systems mentioned are each substituted by R radicals; and where one or more CH₂ groups in the alkyl, alkoxy, alkenyl and alkynyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R radicals; and where one or more CH₂ groups in the alkyl, alkoxy, alkenyl and alkynyl groups mentioned may be replaced by —R⁵C═CR⁵—, —C═C—, Si(R⁵)₂, C═O, C═NR⁵, —C(═O)O—, —C(═O)NR⁵—, NR⁵, P(═O)(R⁵), —O—, —S—, SO or SO₂;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C═C—, Si(R⁵)₂, C═O, C═NR, —C(═O)O—, —C(═O)NR⁵—, NR⁵, P(═O)(R⁵), —O—, —S—, SO or SO₂:
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C═C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; and the alkyl, alkoxy, alkenyl and alkynyl groups, aromatic ring systems and heteroaromatic ring systems mentioned may be substituted by one or more radicals selected from F and CN;
n is 0 or 1, where, when n=0, the G group and the nitrogen atom in formula (I) are bonded directly to one another.

18. The electronic device as claimed in claim 17, wherein formula (I) conforms to one of formulae (I-1-1), (I-1-2), (I-1-3), (I-1-4), (I-3-1), (I-3-2) 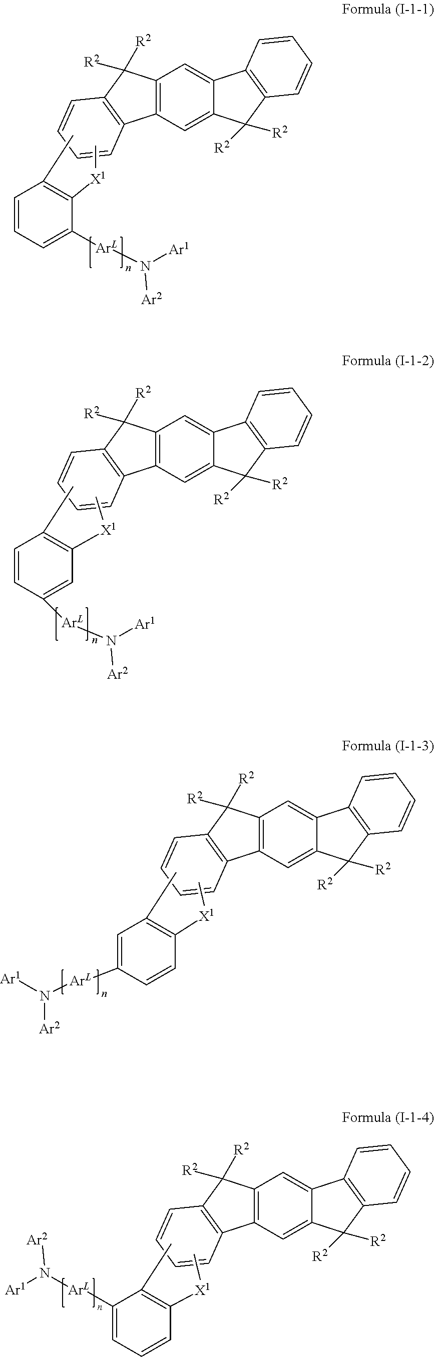 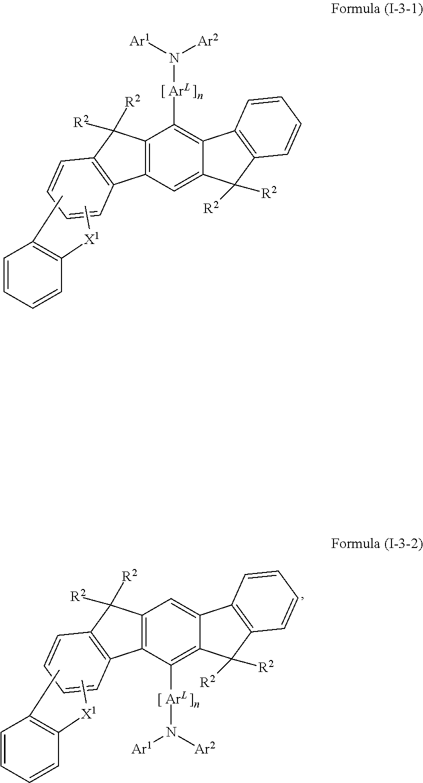
where the variables that occur are as defined in claim 17, and where the other unoccupied positions on the benzene rings are each substituted by an R¹ radical that, in this case, is preferably H.

19. The electronic device as claimed in claim 17, wherein formula (I) conforms to one of formulae (I-4-1), (I-4-2) and (I-4-4), especially formula (I-4-1) 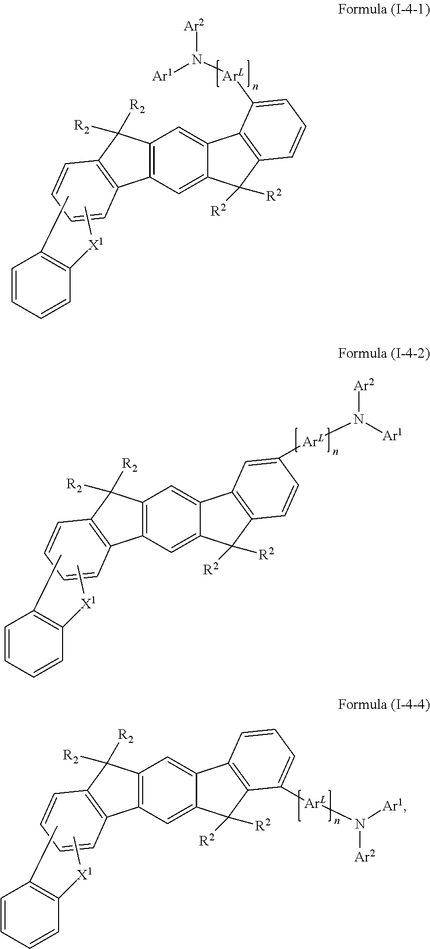
where the variables that occur are as defined in claim 17, and where the other unoccupied positions on the benzene rings are each substituted by an R¹ radical that, in this case, is preferably H.

20. The electronic device as claimed in claim 17, wherein formula (I) conforms to formula (I-4-3) 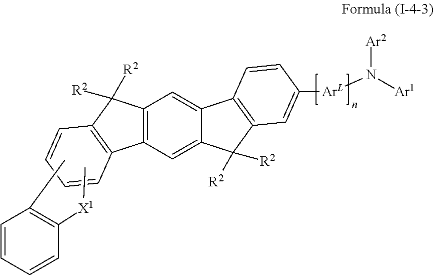
where the variables that occur are as defined in claim 17, and where the other unoccupied positions on the benzene rings are each substituted by an R¹ radical that, in this case, is preferably H, and
where at least one of conditions a) and b) exists:
a) R² is the same or different at each instance and is selected from straight-chain alkyl groups having 1 to 20 carbon atoms and branched or cyclic alkyl groups having 3 to 20 carbon atoms; where two or more R² radicals may be joined to one another and may form a ring; where the alkyl groups mentioned may each be substituted by R⁵ radicals; and where one or more CH₂ groups in the alkyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
b) index n is 1.

21. The electronic device as claimed in claim 17, wherein Ar^{L} is selected from phenyl, biphenyl, naphthyl and fluorenyl, each of which is substituted by R³ radicals; and is preferably selected from phenyl substituted by R³ radicals.

22. The electronic device as claimed in claim 17, wherein at least one of the Ar¹ and Ar² groups is an aromatic ring system which has at least 12 aromatic ring atoms and is substituted by R⁴ radicals.

23. The electronic device as claimed in claim 17, wherein R² is the same or different at each instance and is selected from straight-chain alkyl groups having 1 to 20 carbon atoms, and branched or cyclic alkyl groups having 3 to 20 carbon atoms.

24. The electronic device as claimed in claim 17, wherein the following definitions of the variables exist in combination:
G is a group of formula (G) which is bonded to the remainder of the formula (I) via one of the unoccupied positions on the benzene rings, where the other unoccupied positions on the benzene rings are each substituted by an R¹ radical;
X¹ is O or S;
X² is C(R²)₂;
X³ is C(R²)₂;
Ar^{L} is selected from phenyl, biphenyl, naphthyl and fluorenyl, which are each substituted by R³ radicals;
Ar¹, Ar² is the same or different and is an aromatic ring system which has 6 to 40 aromatic ring atoms and is substituted by R⁴ radicals, or a heteroaromatic ring system which has 5 to 25 aromatic ring atoms and is substituted by R⁴ radicals;
R¹ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals;
R² is the same or different at each instance and is selected from straight-chain alkyl groups having 1 to 20 carbon atoms and branched or cyclic alkyl groups having 3 to 20 carbon atoms;
R³ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁵ radicals;
R⁴ is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R radicals;
R is the same or different at each instance and is selected from H, D, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁶ radicals, and where R⁶ radicals are H;
n is 0 or 1.

25. The electronic device as claimed in claim 17, wherein, in the compound of the formula (I), the G group is selected from one of the following formulae: 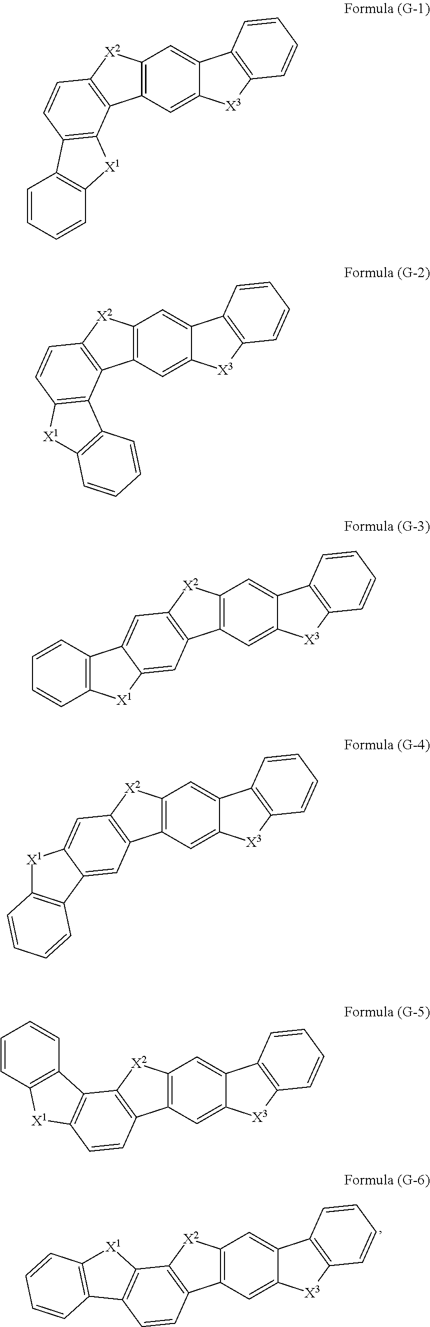
where the G group is bonded to the remainder of the formula (I) via one of the unoccupied positions on the benzene rings, where the other unoccupied positions on the benzene rings are each substituted by an R¹ radical, and where, among the formulae (G-1) to (G-6), preference is given to formulae (G-1) and (G-2), and preference is given in particular to formula (G-1).

26. The electronic device as claimed in claim 17, wherein the layer comprising the compound of the formula (I) is selected from a hole injection layer, a hole transport layer and an electron blocker layer.

27. A compound of a formula (I) 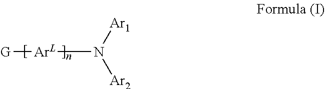
where the variables G, X¹, X², X³, Ar^{L}, Ar¹, Ar², E, R¹, R³, R⁴, R⁵, R⁶ and n are as defined in claim 17, and in addition:
R² is the same or different and is selected from straight-chain alkyl groups having 1 to 20 carbon atoms and branched or cyclic alkyl groups having 3 to 20 carbon atoms; where two or more R² radicals may be joined to one another and may form a ring; where the alkyl groups mentioned are each substituted by R⁵ radicals; and where one or more CH₂ groups in the alkyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂; preferably, it is the same or different and is selected from straight-chain alkyl groups having 1 to 20 carbon atoms and branched or cyclic alkyl groups having 3 to 20 carbon atoms; more preferably, it is methyl.

28. A process for preparing a compound as claimed in claim 27, wherein there is at least one sequence of process steps a) followed by b), where process step a) comprises a reaction in which a carboxylic ester group that binds to a biphenyl unit in the ortho position to the bond between the two benzene rings of the biphenyl unit is converted to a tertiary alcohol group by addition of a metal organyl, preferably Grignard reagent, and where process step b) comprises a reaction in which the tertiary alcohol group obtained in process step a) enters into a ring closure reaction under acidic conditions, especially acidic ion exchange resin, for example Amberlyst-15, such that a fluorenyl unit is formed from the biphenyl unit.

29. An oligomer, polymer or dendrimer containing one or more compounds as claimed in claim 27, wherein the bond(s) to the polymer, oligomer or dendrimer may be localized at any desired positions substituted by R¹, R², R³ or R⁴ in formula (I).

30. A formulation comprising at least one compound as claimed in claim 27 or at least one polymer, oligomer or dendrimer containing the one or more compounds, wherein the bond(s) to the polymer, oligomer or dendrimer may be localized at any desired positions substituted by R¹, R², R³ or R⁴ in formula (I), and at least one solvent.

31. An electronic device comprising at least one compound as claimed in claim 27, or at least one polymer, oligomer or dendrimer containing the one or more compound, wherein the bond(s) to the polymer, oligomer or dendrimer may be localized at any desired positions substituted by R¹, R², R³ or R⁴ in formula (I).

32. A method comprising including the compound as claimed in claim 27 in an electronic device.
